(19) United States
(12) Patent Application Publication
(10) Pub. No.: US 2012/0296403 A1
(43) Pub. Date: Nov. 22, 2012

(54) METHODS AND COMPOUNDS FOR MUSCLE GROWTH

(75) Inventors: David Glass, Cortlandt Manor, MA (US); Jun Shi, Needham, MA (US)

(73) Assignee: NOVARTIS AG, Basel (CH)

(21) Appl. No.: 13/576,308

(22) PCT Filed: Feb. 8, 2011
(86) PCT No.: PCT/EP2011/051825

§ 371 Date: Jul. 31, 2012

Related U.S. Application Data
(60) Provisional application No. 61/303,027, filed on Feb. 10, 2010.

(51) Int. Cl. A61K 31/713; G01N 33/577; C07H 21/02; A61K 38/02; C07K 2/00; A61K 39/395; C07K 16/18; A61K 38/21; A61K 31/56; A61K 38/27; A61K 36/16; A61K 31/7048; A61K 38/18; A61K 38/28; A61K 38/30; A61K 38/48; A61K 38/45; A61K 38/19; A61K 38/44; A61P 21/00; A61P 21/06; A61P 35/00; A61P 29/00; A61P 37/04; A61P 3/10; A61P 9/12; A61P 3/06; A61P 25/16; A61P 25/00; A61P 1/16; A61P 1/14; A61P 13/12; A61N 1/05; G01N 33/53

(52) U.S. Cl. 607/115; 435/7.1; 435/7.9; 514/44 A; 536/24.5; 514/1.1; 530/350; 424/158.1; 530/389.2; 424/85.4; 514/171; 514/11.3; 424/752; 514/27; 514/7.7; 514/6.5; 514/8.6; 424/94.64; 424/94.5; 424/85.1; 424/94.4

(57) ABSTRACT

The disclosure relates to treating muscle wasting-associated disorders in a patient, using a therapeutically effective amount of an antagonist of Fbxo40, wherein the antagonist reduces the expression, level or activity of Fbxo40. The Fbxo40 antagonist increases muscle mass, or prevents, limits or reduces muscle mass loss, in the patient. The Fbxo40 antagonist can be a low molecular weight (LMW) compound, a protein, an antibody, or an inhibitory nucleic acid, such as a siRNA. The disclosure also relates to methods of screening for antagonists of Fbxo40, and methods of diagnosing or monitoring levels of muscle mass maintenance, loss or increase.

This application is a U.S. National Phase filing of International Application No. PCT/EP2011/051825 filed 8 Feb. 2011, which claims priority to U.S. Provisional Application Ser. No. 61/303,027, filed 10 Feb. 2010, the contents of which are incorporated herein by reference in their entirety.

### FIELD OF THE INVENTION

The present disclosure relates to methods of treating muscle wasting-associated disorders using a therapeutically effective amount of an antagonist of Fbxo40, wherein the antagonist reduces the expression, level or activity of Fbxo40. The Fbxo40 antagonist increases muscle mass or prevents, limits or reduces the loss of muscle mass in a patient with or at risk for a muscle wasting-associated disorder. The Fbxo40 antagonist can comprise a low molecular weight (LMW) compound, a protein, an antibody, and/or an inhibitory nucleic acid, such as a siRNA. The disclosure also encompasses methods of screening compositions for the ability to antagonize Fbxo40 and increase muscle mass or prevent the loss of muscle mass in an individual. The disclosure further encompasses diagnostic methods for detecting Fbxo40, wherein an elevated level of Fbxo40 is associated with a muscle-wasting disorder, or a risk thereof.

### BACKGROUND OF THE INVENTION

Muscle loss, wasting or atrophy is associated with many different disorders. Sarcopenia is age-related muscle loss. Cachexia is severe body wasting, associated with weight loss, anorexia, asthenia, anemia and muscle wasting. Decreased muscle mass and integrity is also associated with AIDS wasting syndrome, denervation, injury, cancers, and various other disorders.

Several types of treatment have been suggested to increase muscle mass, including those addressing components of the IGF1 signal pathway, which culminates in protein synthesis and muscle hypertrophy. However, many of the players in this pathway also function in other pathways, or are distributed in tissues other than muscle tissues. This may make developing medicaments antagonizing these components difficult.

There exists the need for a new targeted, specific treatment for muscle loss. Such a therapy can operate alone, or in concert with available therapies.

### BRIEF SUMMARY OF THE INVENTION

The present disclosure provides the use of antagonists to Fbxo40 for increasing muscle mass in individuals in need thereof. The disclosure also provides methods for screening compositions for the ability to antagonize Fbxo40 and increase or maintain muscle mass, or prevent, limit or reduce the loss thereof. The disclosure further encompasses diagnostic methods for detecting Fbxo40, wherein an elevated level of Fbxo40 is associated with a muscle-wasting disorder, or a risk thereof.

As shown in FIG. 1, Fbxo40 is a player in the IGF1 signaling pathway, which promotes muscle hypertrophy. IGF1, via its receptor, activates IRS1 (insulin receptor substrate 1), which leads through various steps to protein synthesis and muscle growth. Fbxo40 antagonizes this function by facilitating the ubiquitination and degradation of IRS1. Inhibition of Fbxo40 allows continued activity of IGF1 and IRS1, which enhances muscle hypertrophy.

Unlike many of the other components of the IGF1 pathway, Fbxo40 is only known to participate in this one pathway. In addition, unlike many other IGF1 pathway players, Fbxo40 is only highly expressed in heart and skeletal muscles. Thus, inhibiting Fbxo40 provides a specific, targeted approach to increasing muscle mass. Furthermore, inhibiting Fbxo40 allows the pathway to be sustained, thus potentiating the ability of IGF1 to promote muscle growth. In short, administration of Fbxo40 inhibitors can act alone or in conjunction with other therapies (including, but not limited to, the administration of IGF1) in facilitating muscle hypertrophy.

In one particular specific embodiment, the present disclosure encompasses methods and compositions related to antagonists to Fbxo40, which improve muscle growth, or prevent, limit or reduce the loss thereof.

In one particular specific embodiment, the present disclosure encompasses methods of identifying compositions comprising an antagonist to Fbxo40, wherein the composition is useful for improving or maintaining muscle mass, or preventing, limiting or reducing the loss thereof.

In another particular specific embodiment, the present disclosure also encompasses diagnostic methods for detecting Fbxo40, wherein an elevated level of Fbxo40 is associated with a muscle-wasting disorder, or a risk thereof.

### BRIEF DESCRIPTION OF THE DRAWINGS

FIG. 1 diagrams the IGF1 signaling pathway, which leads to protein synthesis and muscle hypertrophy.

FIG. 2 is a cartoon showing the physical arrangement of IRS1 and the SCF^{Fbxo40} complex. The complex comprises Fbxo40, Skp1, Cullin1, and Rbx1. Fbxo40 binding to phosphorylated IRS1 brings IRS1 into the complex, where it is ubiquitinated by Rbx1.

FIG. 3 shows that Fbxo40 is highly expressed in heart muscle and skeletal muscle, but not in adipose, bladder, brain, cervix, colon, esophagus, kidney, liver, lung, ovary, placenta, prostate, small intestine, spleen, testes, thymus, thyroid, or trachea tissues.

FIGS. 4A, 4B, and 4E demonstrates that, in C2C12 myotubes, IRS1 is degraded upon IGF1 treatment. 4C and 4D demonstrated that, in C2C12 myotubes, IRS1 is ubiquitinated and this ubiquitination increased with IGF1 treatment.

FIGS. 5A to 5G show that IRS1 is targeted by the Skp1-Cullin1-Rbx1 complex, but not Cullin2 containing complex.

FIGS. 6A to 6D show that Fbxo40 associates with the Skp1-Cullin1-Rbx1 complex and targets IRS1 for degradation.

FIGS. 7A and 7B show that partial knockdown of Rbx1 potentiates the hypertrophic action of IGF1 in C2C12 myotubes.

FIG. 8A show that Fbxo40 expression is detectable at later stages of differentiation

FIG. 9A shows that knockdown of Fbxo40 results in the generation of dramatically larger myotubes even without additional IGF1 treatment.

FIG. 9B shows the quantification of myotube diameter after knockdown of Fbxo40.

FIG. 9C shows that, when IRS1 is knocked down together with Fbxo40, an approximately 20% increase in myotube diameter occurs.

FIG. 9D shows that IRS1 protein is higher in siRbx1 and siFbxo40 electroporated samples than siCON samples.

FIG. 9E shows that larger muscle fibers are also observed with Fbxo40 knockdown compared to siCON electroporated contralateral legs.

### DETAILED DESCRIPTION OF THE INVENTION

The present disclosure is based on the idea that antagonizing Fbxo40 increases muscle mass and/or prevents, limits or reduces the loss of muscle mass. We provide herein a Fbxo40 antagonist, which can be used to treat sarcopenia, cachexia and other muscle loss-associated-disorders, such as those listed herein and those that are known or become known in the art. This antagonist can be a low-molecular weight compound (LMW), an antibody, or an inhibitory nucleic acid such as a siRNA, or any other composition which antagonizes Fbxo40. The Fbxo40 antagonist increases muscle mass or prevents, limits or reduces the loss of muscle mass. The antagonist to Fbxo40 can be administered alone or in conjunction with other therapies. The present disclosure also encompasses methods of screening compositions for the ability to antagonize Fbxo40 and increase muscle mass or prevent, limit or reduce the loss of muscle mass. The present disclosure also encompasses diagnostic methods for detecting Fbxo40, wherein an elevated level of Fbxo40 is associated with a muscle-wasting disorder, or a risk thereof.

A human patient with a muscle wasting-associated disorder would be able to maintain muscle mass, or have a higher level of muscle mass and/or strength as a result, direct or indirect, of being administered the Fbxo40 antagonist. The antagonist can also be administered to non-human animals such as, e.g., cows, pigs, chickens, dogs, cats, and other animals, to increase their muscle mass.

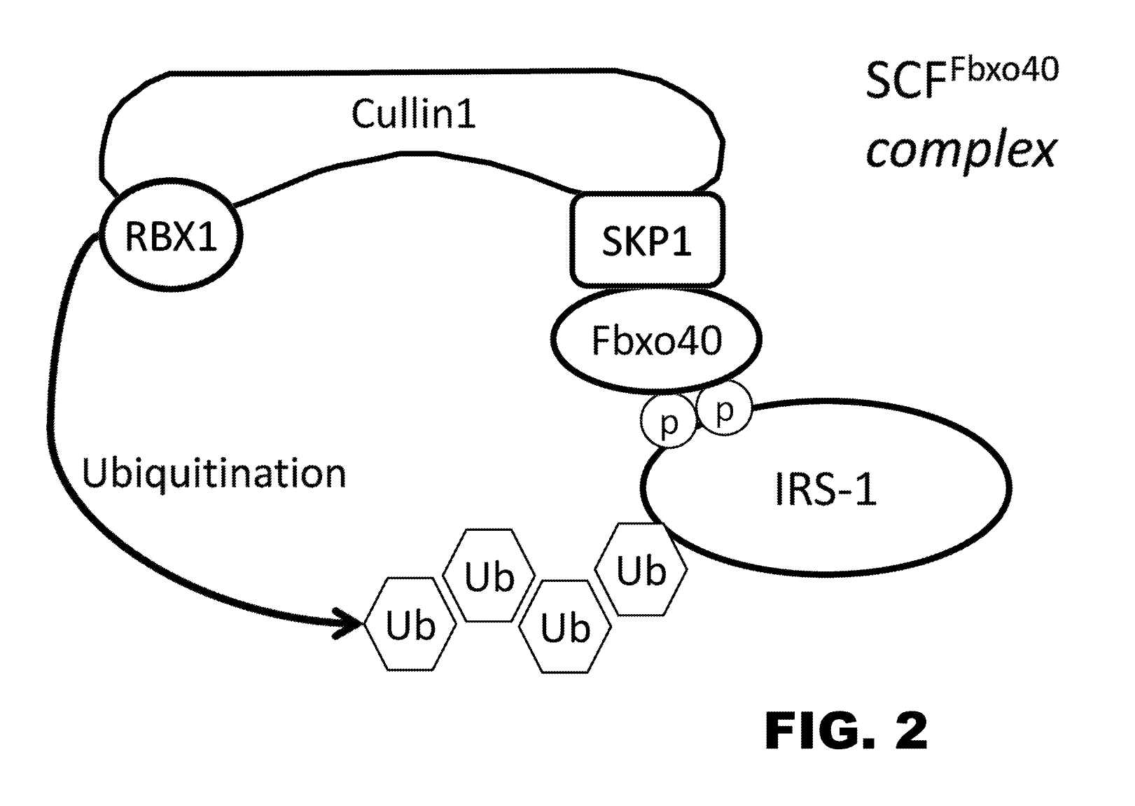

Without wishing to be limited to a particular theory, the inventors suggest that Fbxo40 mediates an activity through direct contact with IRS1 (insulin receptor substrate 1), as shown in FIG. 2. In the IGF1 signaling pathway, IRS1 activation leads to muscle growth. Fbxo40 antagonizes this activity. Fbxo40 brings IRS1 into association with the SCF^{Fbxo40} complex (comprising Fbxo40, Skp1, Cullin1 and Rbx1). In this complex, Rbx1 ubiquinitinates IRS1, marking it for degradation. Inhibition of Fbxo40 prevents the ubiquinitination of IRS1, allowing IRS1 to continue to activate muscle growth.

Accordingly, in one particular specific embodiment, the present disclosure encompasses a method of increasing muscle mass or preventing the loss of muscle mass in an individual, comprising administering to the individual a therapeutically effective amount of an antagonist of Fbxo40. In one embodiment the disclosure provides Fbxo40 antagonists for use in therapy or as medicament for use in the treatment of a pathological disorder.

In one particular specific embodiment, the present disclosure encompasses a method of screening compositions for the ability to increase muscle mass or prevent, limit or reduce the loss of muscle mass in an individual, comprising:
(a) ascertaining the level or activity of Fbxo40 in a cell from the individual,
(b) optionally, treating the cell with a composition comprising an antagonist to Fbxo40, and
(c) optionally, ascertaining the level or activity of Fbxo40 in the cell again,
   wherein an elevated level of Fbxo40 relative to a control is an indication that the subject has or is at risk of developing a muscle-wasting disorder, and wherein an ability of the composition to decrease the level or activity of Fbxo40 is correlated to the ability to increase muscle mass or prevent, limit or reduce the loss of muscle mass in an individual.

In one embodiment of this method, the individual is afflicted with a muscle wasting-associated selected from: cachexia, cancer, tumor-induced weight loss, sepsis, chronic heart failure, rheumatoid arthritis, acquired immune deficiency syndrome, sarcopenia, diabetes, hypertension, high levels of serum cholesterol, high levels of triglycerides, Parkinson's disease, insomnia, drug addiction, pain, insomnia, hypoglycemia, compromised liver function, cirrhosis, gall bladder disorders, chorea, dyskinesia, kidney disorder, and/or uremia.

In one embodiment of this method, the antagonist reduces the level, expression or activity of Fbxo40.

In one particular specific embodiment, the present disclosure encompasses a method of method of diagnosing or monitoring the level of muscle mass increase or maintenance in an individual, comprising:
(a) ascertaining the level or activity of Fbxo40 in a cell from the individual,
(b) optionally, treating the cell with a composition comprising an antagonist to Fbxo40, and
(c) optionally, ascertaining the level or activity of Fbxo40 in the cell again,
   wherein an elevated level of Fbxo40 relative to a control is an indication that the subject has or is at risk of developing a muscle-wasting disorder, and wherein an ability of the composition to decrease the level or activity of Fbxo40 is correlated to the ability to increase muscle mass or prevent, limit or reduce the loss of muscle mass in an individual.

In one embodiment of this method, the individual is afflicted with a muscle wasting-associated disorder selected from: cachexia, cancer, tumor-induced weight loss, sepsis, chronic heart failure, rheumatoid arthritis, acquired immune deficiency syndrome, sarcopenia, diabetes, hypertension, high levels of serum cholesterol, high levels of triglycerides, Parkinson's disease, insomnia, drug addiction, pain, insomnia, hypoglycemia, compromised liver function, cirrhosis, gall bladder disorders, chorea, dyskinesia, kidney disorder, and/or uremia.

In one embodiment of this method, the antagonist reduces the level, expression or activity of Fbxo40.

In one particular specific embodiment, the present disclosure encompasses a method of increasing muscle mass or preventing the loss of muscle mass in an individual, comprising administering to the individual a therapeutically effective amount of an antagonist of Fbxo40.

In one embodiment of this method, the individual is afflicted with a muscle wasting-associated disorder selected from: cachexia, cancer, tumor-induced weight loss, sepsis, chronic heart failure, rheumatoid arthritis, acquired immune deficiency syndrome, sarcopenia, diabetes, hypertension, high levels of serum cholesterol, high levels of triglycerides, Parkinson's disease, insomnia, drug addiction, pain, insomnia, hypoglycemia, compromised liver function, cirrhosis, gall bladder disorders, chorea, dyskinesia, kidney disorder, and/or uremia.

In one embodiment of this method, the method further comprises administering physiotherapy, nutrients, electrical stimulation, electrical neuromuscular stimulators of NMES, neural input to the muscles; and/or one or more of the following: steroid, hormone, growth hormone, growth hormone secretagogue; ibutamoren mesylate (MK-677), gingko biloba extract, flavoneglycoside, ginkgolide, amino acid supplement, leucine, amino acid precursor, leucine precursor, pyruvate and pyruvate metabolite, beta-hydroxy-beta-methylbutyrate, alpha-ketoisocaproate, branched chain amino acid, erythropoietin, opiate, scopolamine, insulin, insulin-like growth factor-1 (IGF1), and/or testosterone; and/or inhibitor of aldosterone, alpha receptor, Angiotensin II, beta receptor, cathepsin B, chymase, endothelin receptor, eukaryotic initiation factor 2-alpha (eIF2-alpha), imidazoline receptor, interferon, MAFbx (Muscle Atrophy F-box), MuRF1 (Muscle RING Finger 1), myostatin, parathyroid hormone related protein (PTHrP) and/or its receptor, proteolysis-inducing factor (PIF), RNA-dependent serine/threonine protein kinase (PKR), tumor necrosis factor alpha (TNF-alpha), and/or xanthine oxidase.

In one embodiment of this method, the antagonist reduces the expression, level, or activity of Fbxo40.

In one embodiment of this method, the antagonist of Fbxo40 is a low molecular weight compound.

In one embodiment of this method, the antagonist is a polypeptide.

In one embodiment of this method, the antagonist of Fbxo40 is a siRNA that binds to a nucleic acid encoding Fbxo40.

In one embodiment of this method, the siRNA is blunt-ended.

In one embodiment of this method, the antagonist of Fbxo40 is an antibody that binds to Fbxo40.

In one particular specific embodiment, the present disclosure encompasses a composition comprising an antagonist of Fbxo40, wherein the antagonist reduces the expression, level or activity of Fbxo40 and increases muscle mass or prevents, limits or reduces the loss of muscle mass.

In one embodiment of this composition, the composition further comprises one or more of the following: steroid, hormone, growth hormone, growth hormone secretagogue; ibutamoren mesylate (MK-677), gingko biloba extract, flavoneglycoside, ginkgolide, amino acid supplement, leucine, amino acid precursor, leucine precursor, pyruvate and pyruvate metabolite, beta-hydroxy-beta-methylbutyrate, alpha-ketoisocaproate, branched chain amino acid, erythropoietin, opiate, scopolamine, insulin, insulin-like growth factor-1 (IGF1), and/or testosterone; and/or inhibitor of aldosterone, alpha receptor, Angiotensin II, beta receptor, cathepsin B, chymase, endothelin receptor, eukaryotic initiation factor 2-alpha (eIF2-alpha), imidazoline receptor, interferon, MAFbx (Muscle Atrophy F-box), MuRF1 (Muscle RING Finger 1), myostatin, parathyroid hormone related protein (PTHrP) and/or its receptor, proteolysis-inducing factor (PIF), RNA-dependent serine/threonine protein kinase (PKR), tumor necrosis factor alpha (TNF-alpha), and/or xanthine oxidase.

In one embodiment of this composition, the antagonist reduces the expression, level or activity of Fbxo40.

In one embodiment of this composition, the antagonist of Fbxo40 is a low molecular weight compound.

In one embodiment of this composition, the antagonist is a polypeptide.

In one embodiment of this composition, the antagonist of Fbxo40 is a siRNA that binds to a nucleic acid encoding Fbxo40.

In one embodiment of this composition, the siRNA is blunt-ended.

In one embodiment of this composition, the antagonist of Fbxo40 is an antibody that binds to Fbxo40.

The Fbxo40 antagonist of the present disclosure can, therefore, be useful to treat muscle wasting associated with disorders including, but not limited to, diabetes, denervation, injury, cardiovascular disease, neural degeneration, and various cancers.

### DEFINITIONS

In order to provide a clear understanding of the specification and claims, the following definitions are conveniently provided below.

By “Fbxo40” is meant a gene or protein which is a member of the Fbox family of genes and proteins, the human homologue of which is represented by Genbank No. NM_{—}016298. The animal homologues of this gene are known in several species: Mouse (Mus musculus): NM_{—}001037321; Rat (Rattus norvegicus): XM_{—}344023; Chimpanzee (Pan troglodytes): NC_{—}006490.2; Rhesus macaque (Macaca mulatta): NC_{—}007859.1; Zebrafish (Danio rerio): BX322577.11 (pseudogene) or XP_{—}694708.3; Wild boar (Sus scrofa): EU743742; Chicken (Gallus gallus): XP_{—}424000.2; and Dog (Canis lupus familiaris): XP_{—}545126.2.

A representative human homologue of Fbxo40 includes, but is not limited to, the following amino acid sequence (SEQ ID NO. 1, Genbank No. NM_{—}016298):

| | |
|---|---|
| 1 | MGKARRSPPG HHRHCEGCFN RHCHIPVEPN TSCLVISCHL LCGATFHMCK EAEHQLLCPL |
| 61 | EQVPCLNSEY GCPLSMSRHK LAKHLQVCPA SVVCCSMEWN RWPNVDSETT LHENIMKETP |
| 121 | SEECLDTALA LQDQKVLFRS LKMVELFPET REATEEEPTM NGETSVEEMG GAVGGVDIGL |
| 181 | VPHGLSATNG EMAELSQEER EVLAKTKEGM DLVKFGQWEN IFSKEHAASA LTNSSASCES |
| 241 | KNKNDSEKEQ ISSGHNMVEG EGAPKKKEPQ ENQKQQDVRT AMETTGLAPW QDGVLERLKT |
| 301 | AVDAKDYNMY LVHNGRMLIH FGQMPACTPK ERDFVYGKLE AQEVKTVYTF KVPVSYCGKR |
| 361 | ARLGDAMLSC KPSEHKAVDT SDLGITVEDL PKSDLIKTTL QCALERELKG HVISESRSID |
| 421 | GLFMDFATQT YNFEPEQFSS GTVLADLTAA TPGGLHVELH SECVTRRHNK SSSAFTFTCN |
| 481 | KFFRRDEFPL HFKNVHTDIQ SCLNGWFQHR CPLAYLGCTF VQNHFRPPGQ KAKVIYSQEL |
| 541 | KTFAIKPEVA PELSEGRKNN HLLGHGGKSQ NSLTSLPLEI LKYIAGFLDS VSLAQLSQVS |
| 601 | VLMRNICATL LQERGMVLLQ WKKKRYSHGG TSWRVHREIW QFSSLFSKIK SWEFNEVTSM |
| 661 | SEHLKSCPFN IVEHKTDPIL LTSMCQPREQ ARESLVSTFR IRPRGRYVS |

The mRNA sequence for human Fbxo40 is readily available, e.g., at GenBank: NM_{—}016298 (SEQ ID NO: 2):

| | |
|---|---|
| 1 | ATTTTTAACT TCGCAACACT TGGACTATTT CTGTTGAAGT TTTCTCTCCT TTCCCTGCCT |
| 61 | TCCCAACAGA ACGCTGTCCT CTACTGCAGC TGATGCAACC CAGCCACCTC CCGGCAATCC |
| 121 | GCTTACTTGC AATCAAGGGT TCAGGTGCAA GCAGATGCTG ACTCAGCCTG TTCCATTAAG |
| 181 | AGCTAAGAAG CAAGAAGAAA TTGGGGCGCC ATGGGGAAAG CCCGCAGATC CCCGCCAGGG |
| 241 | CACCACAGGC ATTGTGAGGG ATGCTTCAAC CGCCACTGCC ACATTCCTGT GGAACCCAAC |
| 301 | ACCTCCTGCC TGGTAATAAG CTGCCACCTG CTCTGTGGTG CCACCTTCCA CATGTGCAAA |
| 361 | GAGGCAGAGC ACCAGCTCCT CTGCCCTTTA GAGCAGGTTC CGTGCCTCAA CTCCGAATAT |
| 421 | GGCTGCCCTC TGTCCATGTC CCGCCACAAA CTGGCCAAGC ACCTGCAGGT GTGCCCCGCC |
| 481 | AGCGTGGTCT GCTGCTCCAT GGAGTGGAAC CGCTGGCCAA ATGTGGACTC TGAAACCACC |
| 541 | CTTCATGAAA ACATCATGAA AGAGACCCCC AGTGAGGAGT GTTTGGACAC AGCCCTGGCC |
| 601 | CTGCAGGATC AGAAGGTCCT CTTCAGATCC TTGAAAATGG TGGAACTTTT CCCAGAAACT |
| 661 | AGAGAGGCTA CTGAGGAGGA ACCAACTATG AATGGTGAAA CCAGTGTGGA GGAAATGGGA |
| 721 | GGAGCAGTGG GTGGAGTGGA TATCGGTTTG GTACCACATG GTCTGTCAGC AACTAATGGG |
| 781 | GAGATGGCAG AGCTAAGTCA AGAAGAACGG GAGGTGCTAG CCAAAACCAA AGAAGGGATG |
| 841 | GACCTGGTCA AGTTTGGCCA GTGGGAAAAT ATTTTCAGCA AAGAGCACGC AGCCTCTGCT |
| 901 | TTAACAAATT CATCAGCGAG CTGTGAGAGC AAGAACAAGA ATGACTCCGA GAAAGAACAG |
| 961 | ATTTCCAGTG GCCATAACAT GGTAGAAGGA GAGGGCGCTC CCAAAAAGAA AGAACCACAG |
| 1021 | GAAAATCAGA AGCAGCAGGA CGTTCGTACA GCCATGGAAA CCACAGGGCT TGCCCCTTGG |
| 1081 | CAGGATGGTG TTCTGGAAAG ACTGAAAACA GCTGTGGATG CAAAGGACTA TAACATGTAT |
| 1141 | CTAGTGCACA ATGGGCGGAT GCTGATACAC TTTGGTCAGA TGCCTGCTTG TACACCCAAG |
| 1201 | GAGAGAGACT TTGTTTATGG CAAGCTGGAG GCTCAGGAAG TTAAGACTGT TTACACCTTC |
| 1261 | AAAGTTCCTG TGAGCTACTG TGGAAAGCGA GCTCGACTTG GAGATGCCAT GTTGAGTTGT |
| 1321 | AAGCCAAGTG AACACAAGGC AGTGGATACT TCAGATTTGG GGATCACTGT GGAGGACCTG |
| 1381 | CCCAAATCAG ATCTCATCAA GACCACCCTC CAGTGTGCTT TGGAAAGAGA ACTCAAAGGC |
| 1441 | CACGTCATCT CTGAATCCAG AAGCATTGAT GGACTGTTCA TGGATTTTGC CACACAAACA |
| 1501 | TACAACTTTG AGCCAGAACA GTTTTCCTCT GGGACAGTGC TGGCTGACCT AACCGCTGCC |
| 1561 | ACCCCAGGGG GACTCCACGT GGAGCTCCAC AGCGAGTGTG TGACCAGGAG ACACAACAAA |
| 1621 | AGCAGCTCTG CCTTCACTTT CACTTGCAAC AAATTCTTCA GGAGGGATGA GTTCCCCCTG |
| 1681 | CACTTCAAGA ATGTCCACAC AGACATTCAG TCATGTCTCA ATGGCTGGTT CCAGCATCGA |
| 1741 | TGCCCCCTCG CCTACTTGGG ATGTACATTT GTTCAAAACC ATTTCCGTCC CCCAGGGCAA |
| 1801 | AAGGCAAAAG TAATCTATAG CCAGGAGCTC AAGACCTTTG CCATTAAGCC GGAGGTTGCT |
| 1861 | CCAGAGCTGA GCGAGGGAAG GAAGAACAAC CATCTTTTGG GTCATGGAGG AAAAAGCCAG |
| 1921 | AATTCTTTAA CCAGCCTGCC CCTGGAGATT TTGAAGTACA TTGCTGGGTT CTTGGACAGC |
| 1981 | GTCAGCCTGG CCCAGCTCTC CCAGGTGTCT GTGCTGATGA GGAATATCTG TGCCACTTTG |
| 2041 | TTACAAGAGA GAGGAATGGT CCTTTTGCAA TGGAAGAAAA AGAGGTATTC CCATGGAGGC |
| 2101 | ACCTCCTGGA GAGTCCACAG AGAGATCTGG CAGTTCAGCA GCCTCTTCTC CAAAATCAAG |
| 2161 | AGCTGGGAGT TTAATGAAGT CACCTCCATG TCTGAGCACC TGAAGTCCTG TCCTTTCAAC |
| 2221 | ATTGTAGAGC ACAAAACTGA CCCGATTCTT TTGACTAGCA TGTGTCAGCC CCGTGAGCAG |
| 2281 | GCCCGAGAGA GCTTAGTCTC CACCTTTAGA ATCAGACCAC GAGGAAGATA CGTCTCCTAA |
| 2341 | AAATTCAGAT GCCACTCGAT GCACCCTTCT TGGATTTCTT CTCGGAGTTC CTGAAGTAGG |
| 2401 | ACAGAGTGTG TGGTTTTGAG GACTCCCTTC TGTAAACTGC CTATTTGCTT ATCGGGGTGT |
| 2461 | ATTGGAACAC GCAATGTCCT TCGAAACCTC AACACGAGGC CTAAGAATTT CCTAAGCCAT |
| 2521 | GTCTTGTACC ATAGTGCCAC ATTGATGACT TGTTTCCTTT TTTCTTTTCT TTTCTTTTCT |
| 2581 | TTTTTCTTTC TTTCTAAAAT AGATTGGTCT GAGAAGAAAA TAAGTAATTT GAGGCCATTT |
| 2641 | GGAAGATGGG CCCAATTTCT TAAGTGATGA GAGAGCACGA AATTCCATAA CCAGTACAGG |
| 2701 | CCTGTGCTTT TACATGGGCT TTTTAGTTCA CAAAGCACTT TCAAATTTAT GGGACAGGAA |
| 2761 | ATGCAGGATG GGACTCCCCA GGGAACGCAG GGTGAAGGGA ACAAAGCTGG AGGCTCTGGA |
| 2821 | GCTGGGTCTG TTTTGACGGT CAAGTCCAGG GCTCATTTTG GTTATTCTAC TGCCTCTAGG |
| 2881 | CCAGGGTAGT CCTAACACAG CCTGACATAG GAGAGCCCCT GGCTGAGCAT GGCAGCCTTG |
| 2941 | AAGACACCAC AGGCCAAAAC ATGAGGGGCA GAAATGGGAT CACAGAGTCT GTTGCTAGAA |
| 3001 | TCTTGGCAAC ATACAGCAGG AAAGCCTTGA TAAATCGGGA GTCCAAAGGA GACACCATAT |
| 3061 | TTATGGAGAA CATTAGGACA AAAAGTCACC AACTTACTTT GTAACATTTT AATAATGACT |
| 3121 | TAAGGGTCAA GATTTTTTTC TTCTGAAAAT TATGTTCTGA GTTAGGCAGA ACATAGCCAT |
| 3181 | GGCCCTGGGC CACCCTGTGC TATCTGAAAT GACCTCAATA CACTAATGCC AACCTCAGCG |
| 3241 | TCATGCCAGA ATGCACAGGG CAGCCCAGGG AGATCACACC TTTGGCAAAG TCCAGACAAG |
| 3301 | GCCCACTGCA GTTCCTATGG CGCCAGTCAC CAGCTCCTAG ACAGCACTTG GGTACCCCAT |
| 3361 | TGGGGTCTTG GAGAGGAAGA CATGTGAACA TAACGGCTCC CTGAAATTGC TCCTACCCAT |
| 3421 | CCATATTTCT GGTCATGCTT TCAGTCTGAC AAAAATGGAT GATACTGCTG TTTTTGGTAA |
| 3481 | CAAACAGTGA ATATTCATAA GAACAAAAGT AAAAGAAAAA AAGACACAGT AGAAACTGGC |
| 3541 | ATCCCCTAAA GCAGGGCTTC TTAGCCTTGG AACTATTGAC ATTTTTAAAT GGATAATTCT |
| 3601 | TTTTTTTTTT TTCTAGGTGG GGAGGGGATG GAGTTCACTC TTGTTGCCCA GGCTGGAGCG |
| 3661 | CAATGACATG ATCTCGGCTC ACCGCAACCT CCGCCTCCTG GGTTCAAGCG ATTCTCCTGC |
| 3721 | CTCAGCCTCC CGAGTAGCTG GGATTACTCG CCTGGCTAAT TTTGTATTTT TAGTAGAGAC |
| 3781 | GGGCTTTCTC CATGTTGTTC AGGCTGGTCT CAAACTCCCG ACCTCAGGTG ACTCGCCCGC |
| 3841 | CTTGGCCTTC CAAAGTGCTG GGTTTACAGG TGTGAGCCAC TGCGCCCTGC CTGAACTGGA |
| 3901 | TAATTCTTTG TTGCAAGGGA CTGTTCTGTG TACTACAGGA TACTTGGCAG CATCCTTGGC |
| 3961 | CTATCCATTA AATGTCAGTA GCACCCCCAC AGTGGCAACA ATCAAAAATG TCACCAGACA |
| 4021 | TTGCTAAATA TTGGGGAGCA AAATGGCTCC CCGTTGAAAA TCCCTAAAGG ATGTCATACT |
| 4081 | AGTGACAATA AGTTAGGATA TGCTTATTTT TTAGTACAGC AAAATCTTAT CGCACATAGC |
| 4141 | TATCCACAAT AGTTATGATT TAATGCAGCT CTTTATTTAT GAAATAGGTT TTAGACATGT |
| 4201 | GGTGATTTTA AGTTGGGAAC CAGAAGGAAA TGATTCGTTT GGTATGGCTT CATGTCCTTC |
| 4261 | AGCCACCCCC AAGAATGTAT CCTTTCAGCT CTCTTTGGTT ATACCTGAAG CCAGGAGCGT |
| 4321 | TGAGTTATTA GCCTTGTGTT TATATTCCTC TCACTGTAAT TGGTGTCATT TTCCCAGCAG |
| 4381 | TCCTAGCAGT CCTCAAGCAA GTGGGAAATC GGAAAAGAAA AGGACAGGCA TTGTAGGGAA |
| 4441 | GCAGAGGATA AAGAATTTAG CCAACAAAAG AAACAATCTA GTCAATCTGG GTGCTTTTAT |
| 4501 | TTCCTGGGTT CTCTCTAAAC ATGGCTCAGA GCTGGTGTAG ATGAAGTAGG TGAAACCTCT |
| 4561 | GAAAAGAGTC TAGAAGGCAG TAGAGCAAGT CCCAGACCAG AAACATGCTC ATCTTTTCAT |
| 4621 | CGTAATGTGC CACTCGGTAC TATTTGGTAA TGTCACTCTA TTTTTCCTAA TCCCATCCTT |
| 4681 | TGGTTTGTAT TTCATATTTG TATATAAGGC ACCATTTTCT AAAAATATGA CTAGGGTGTG |
| 4741 | ACCTAAGGTT TTATTCTGTG AAGATGAGTA ACTGGAAAGA AGCTAACACT GCAGTGGGAA |
| 4801 | GGAAGGAAGA GAGTTGTCCA GGTGGTAGTT CGACGTGTTT TGAATCTAGT CCTTCCTACA |
| 4861 | TGGAGGATAA AAGCTCCTAA AGTCCACTCT GGGTTTGTGA TTTTAATAGA AATAGAAAGG |
| 4921 | GAAACTATAG ACCAATGGAG ATGAAAATCA GGGGCTATCG ACAGATGGAG GAGAAATAAG |
| 4981 | GTGCTACATA GAGAAAGGAA GAGGGCAGAA GGCTTTCCCT TCCCAAACTG GGTGAGCTGG |
| 5041 | GGAAGCCTTG GTTCAGGAGA GTGGCACTGC CCACAACTGC TTTGTGGGTT GTGCACTTCC |
| 5101 | AGCCGCACTC TCCCCCTCCA GTTGCTGCCT TCAGAGCCGT ACTGAAGCAC GAGCTTCAAT |
| 5161 | AAGACAAGCA CACTTCATAG TGAGAGGGCA GCGGTACCAA AGCCTTTCAG AGAGACTATG |
| 5221 | GATTAGACAG AAATGATTTG TGAGAGGAAG CTGGAGTGAA CAGCATGAAC AGCGAGTGTT |
| 5281 | ACCTGACAGA GGCAAGACAG CTAGAAGTGG CTTCAGATTT AGAAACAGCT GAGGGGAGCA |
| 5341 | AAGACGGACT GTGTACACAG GGAGGGAGGA TGTCTATGGG CAGAGCCCTT GGTGAGTATC |
| 5401 | ATCACCAAGA AAGGCAGTCC AGAGTAGAGA TCAGCCGAAT ATGGAGGCTG AGGTCTGTAG |
| 5461 | AACTGGGCCA GAGAGGACCT TACTGCCTTA GTAGCATAAG GGTCTGGAAA AGAAGTTTCT |
| 5521 | ATCTCACAAC AAAGGAAAAA GTGAAAAGCA AGGTGGAACT TGAAGATACG TCACGAAAAT |
| 5581 | CACTATAAAA GTCTGATTTA TGTGTGATGT CAAATCAAAC TGAAATGAAG AATGAGATTG |
| 5641 | AGTATATCTG TGGTGACTGA CCTCTGTATA CTAGAAACCT CAACATCTCT AGAAGAGGAA |
| 5701 | ATAAAAGCTG CTTTGCACTC TG |

References for this sequence are: Need et al. 2009 Hum. Mol. Genet. 18 (23), 4650-4661; Ye et al. 2007 Gene 404 (1-2), 53-60 (2007); Jin et al. 2004 Genes Dev. 18 (21), 2573-2580 (2004).

Fbxo40 is a member of the family of F-box proteins, which each contain at least one F-box motif, a protein structural motif of about 50 amino acids that mediates protein-protein interactions. See, for example, Bai et al. 1996 Cell 86: 263-74; Kipreos et al. 2000 Genome Biol. 1(5): REVIEWS3002; Craig et al. 1999 Prog. Biophys. Mol. Biol. 72: 299-328; and Ye et al. 2007 Gene 404:53-60. The F-box motif of Fbxo40 interacts directly with protein Skp1.

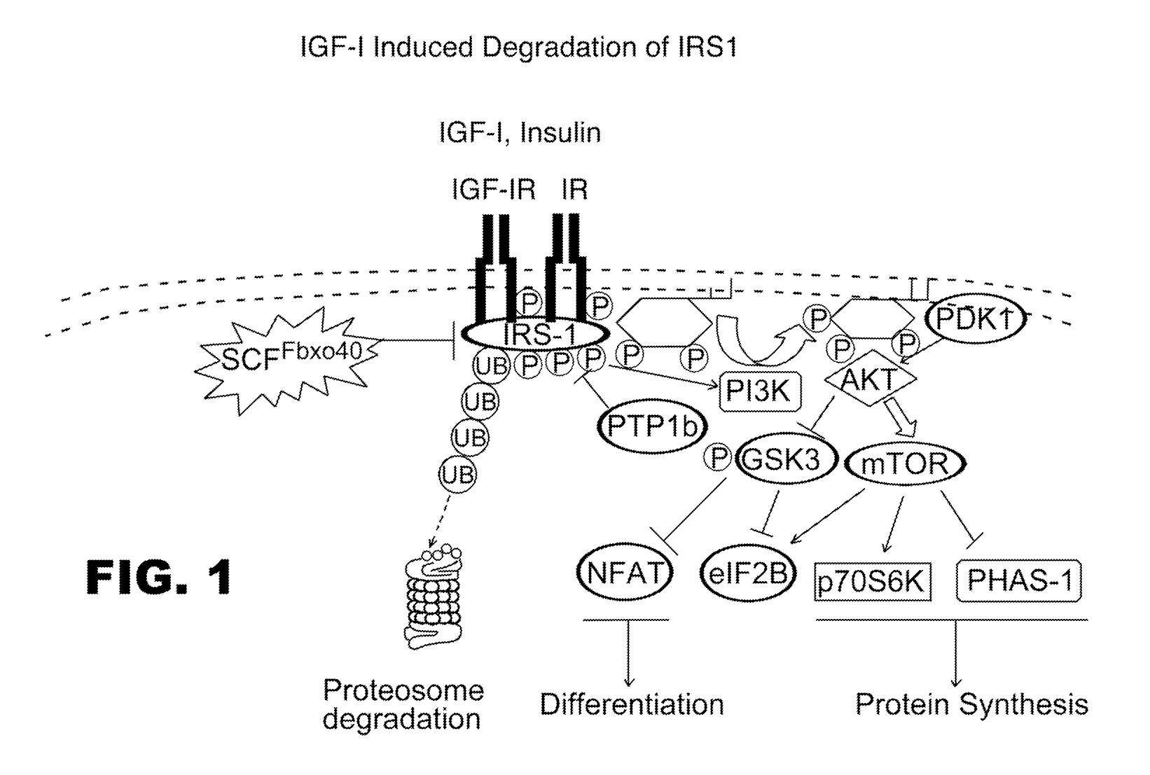

As mentioned above and as diagrammed in FIG. 1, Fbxo40 is involved in the IGF1 signaling pathway. In this pathway, insulin and IGF1 bind to the insulin receptor (IR) and IGF1 receptor (IGF1R), respectively; IGF1 binds to both receptors and has a much higher affinity for IGF1R. This binding activates the intrinsic tyrosine kinase activity of the receptor, which autophosphorylates the triple tyrosine cluster in the activation loop of the kinase domain and tyrosine-phosphorylates IRS1. Phosphorylated IRS1 binds the p85α regulatory subunit of the class IA phosphatidylinositol 3-kinase (PI3K) and activates PI3K. PI3K catalyzes the phosphorylation of the 3-OH position of myo-inositol lipids. PIP3 (phosphatidylinositol-3,4,5-triphosphate) recruits PH domain-containing molecules, such as PDK1 (3-phosphoinositide dependent protein kinase-1, a master kinase) and Akt (a key protein kinase), to the cell membrane, with subsequent phosphorylation and activation of Akt by PDK1. Akt phosphorylates and inactivates glycogen synthase kinase-3 (GSK3). GSK3 is a serine/threonine protein kinase that phosphorylates and inactivates glycogen synthase, NFAT (nuclear factor of activated T-cells, a transcription factor), and eIF2B (guanine nucleotide exchange factor for eukaryotic initiation factor 2). Activated Akt can also activate mTOR (a key serine/threonine kinase), which in turn activates p70S6K and inactivates PHAS-1 (4E-BP) and ultimately leads to protein synthesis and muscle hypertrophy.

IGF1-induced IRS1 phosphorylation can also target IRS1 to be ubiquitinated by SCF^{Fbxo40} and degraded in the proteosome.

Factors which have a negative influence on protein synthesis and muscle hypertrophy are: PTP1b (a protein tyrosine phosphatase), GSK3, and PHAS-1. The other factors have a positive effect on protein synthesis and muscle hypertrophy: PI3K, NFAT, eIF2B, Akt, mTOR, PDK1, and p70S6K.

In this pathway, Fbxo40 antagonizes IRS1. As shown in FIG. 2, Fbxo40 binds to IRS1, bringing it into the SCF^{Fbxo40} complex. Note that the encircled “p” symbols indicate that IRS1 is phosphorylated, though it is speculated but not yet clear if phosphorylation of IRS1 is directly involved in binding to Fbxo40. The SCF^{Fbxo40} complex comprises Skp1, Cullin1 and Rbx1 (RING-box protein 1). While bound in the complex, IRS1 is ubiquitinated (“Ub”) and marked for degradation by Rbx1. Inhibition of Fbxo40 prevents association of IRS1 with the SCF^{Fbxo40} complex, and thus prevents ubiquinitination and degradation of IRS1. This allows continued activity of IRS1 in promoting muscle growth. Inhibiting Fbxo40 thus allows muscle hypertrophy.

Unlike Fbxo40, the other components of the complex (Skp1, Cullin1, and Rbx1) are each involved in many other pathways. This makes Fbxo40 uniquely suited for targeting.

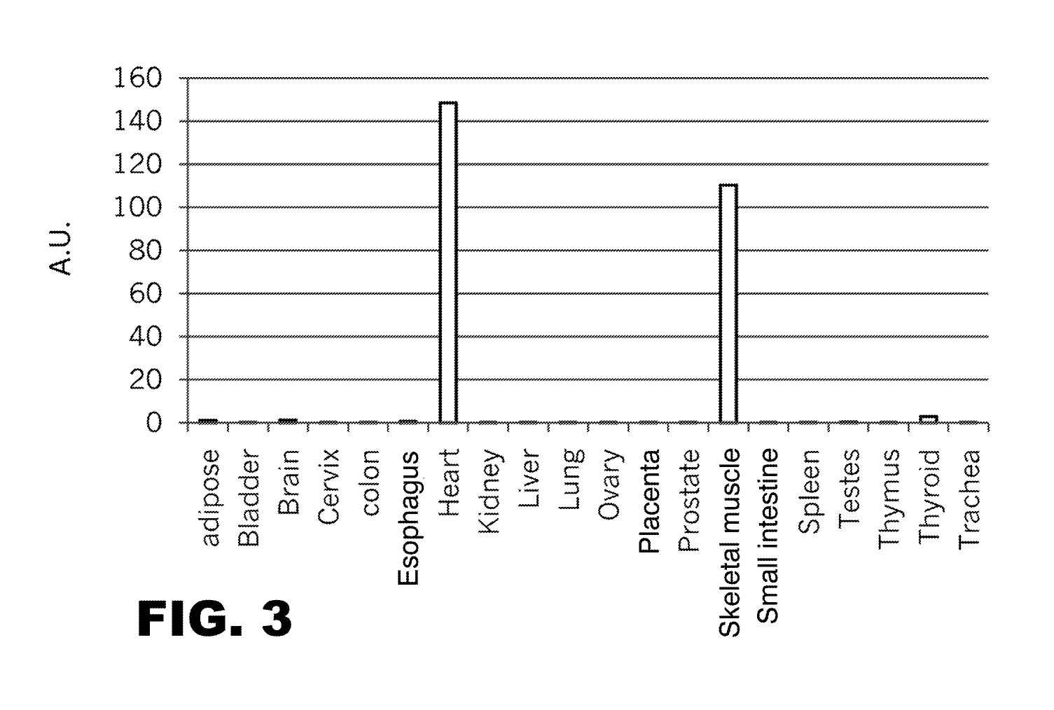

In addition, unlike IGF1, Fbxo40 is only highly expressed in heart and skeletal muscle tissues. Ye et al. 2007 showed that Fbxo40 was not expressed in several tissue types. We show additional data in FIG. 3 that Fbxo40 is not expressed in adipose, bladder, brain, cervix, colon, esophagus, kidney, liver, lung, ovary, placenta, prostate, small intestine, spleen, testes, thymus, thyroid, or trachea tissues. In this Figure, “A.U.” indicates relative arbitrary units. This high tissue-specificity also makes Fbxo40 a desirable target for increasing muscle growth.

### Muscles and Muscle Wasting-Associated Disorders

An antagonist to Fbxo40 can be administered, directly or indirectly, to muscles and muscle tissues of individuals or patients suffering from or at risk for a muscle wasting-associated disorder, and the antagonist can increase the muscle mass or prevent or slow the decrease of muscle mass in such individuals and patients.

By “muscle” is meant any of various contractile tissues, including skeletal, smooth and cardiac muscle; including both voluntary and involuntary muscle, also including both slow and fast twitch muscle. The antagonists of the present disclosure are particularly useful for promoting the growth of or preventing the loss of cardiac and skeletal muscles.

By “muscle wasting-associated disorder” is meant any condition associated with loss of muscle tone or mass. These conditions include, but are not limited to, sarcopenia, cachexia, AIDS wasting syndrome, muscular dystrophy (including Duchenne muscular dystrophy syndrome and Becker's muscule dystrophy syndrome), muscular atrophy, neuromuscular diseases, anorexia, motor neuron diseases, diseases of neuromuscular junction, inflammatory myopathies, other conditions or diseases associated with decreased muscle mass, and other related diseases. These disorders also include chronic or acute “deconditioning,” as may occur from immobilization or inactivity, such as associated with illness or injury, or the rigors of air travel and space travel. Muscle wasting, including muscle atrophy, can also occur as a consequence of denervation, injury, joint immobilization, enforced bed rest (disuse atrophy), glucocorticoid treatment, sepsis, unweighting, cancer and aging. Jagoe et al. 2001 Curr. Opin. Clin. Nutr. Metab. Care 4: 183. In addition there are a variety of rare forms of myopathy (disorders of carbohydrate metabolism, disorders of lipid metabolism, lysosmal myopathies, inclusion body myopathies, distal myopathies, autoimmune inflammatory myopathies etc) that result in severe pain, weakness, fatigue and disability.

Cachexia is a common feature of many illnesses, including cancer, chronic obstructive pulmonary disease (COPD), sepsis, chronic heart failure, rheumatoid arthritis, and acquired immune deficiency syndrome (AIDS). Certain tumors induce cachexia through production of a 24 kDA glycoprotein called proteolysis-inducing factor (PIF). U.S. Patent App. 20090105123. Cachexia can also occur idiopathically.

Cachexia is characterized by marked weight loss, anorexia, asthenia, and anemia. Cachexia can also have the symptoms of loss of appetite, weakness, compromised immune function and electrolyte imbalance. Muscle mass loss can be the result of many factors, including decreased rate of protein synthesis with normal muscle degradation, increased degradation with normal synthesis, or a combination of both reduced synthesis and increased degradation. Maintenance of muscle mass depends on proper nutrition, neural input, and hormonal state.

Sarcopenia is a muscular affliction which afflicts most older people and manifests as a reduction in muscle mass with age. Sarcopenia is related to frailty, fractures and falls that lead to morbidity and mortality. Baumgartner et al. (1998 Am. J. Epidemiol. 147: 755-63; 149: 1161) defined sarcopenia as appendicular skeletal muscle mass (kg/height²) being less than two standard deviations below the mean of a young reference group.

In addition, the patient may be suffering from one or more of the following: alcohol addiction, high levels of serum cholesterol, chorea, diabetes, drug addiction, dyskinesia, gall bladder disorders, chronic heart failure, hypertension, Huntington's Disease, hypoglycemia, an infection (including chronic infection such as pneumonia), insomnia, tumor-induced weight loss, a kidney disorder, including uremia, compromised liver function, including cirrhosis, bone loss (e.g., osteoporosis), disease or damage, pain, Parkinson's disease, pulmonary disease (including chronic obstructive pulmonary disease), rheumatoid arthritis, sepsis, high levels of triglycerides, and inflammatory condition (including chronic inflammation, including inflammatory bowel disease).

Some aspects of the biochemistry of muscle mass loss have been explored. Cachectin, believed to be a causative agent of cancer cachexia, is identical to tumor necrosis factor (TNF). It has been found that cytokines (e.g., interleukin, IL-1, IL-6, LIF, IFN, etc.) also have the same actions as cachectin. Thus, without being bound by any particular theory, applicants note that cachexia may be induced by composite action of multiple factors.

The OCC-1 cell line, derived from human oral cavity carcinoma, produces various liquid factors involved in cancer cachexia. Nude mice implanted with OCC-1 cells develop various syndromes, including cachexia. Kajimura et al. 1996 Cancer Chemother. Pharmacol. 38 Suppl. S48-52; Tanaka et al. 1996 Jpn. J. Clin. Oncol. 26: 88-94. OCC-1 cells implanted into nude mice are believed to produce various cytokines (e.g., G-CSF, IL-6, LIF, IL-11, and PTHrP) that act compositely to cause the symptoms.

Example muscular dystrophies that can be treated with a composition of this disclosure include: Duchenne Muscular Dystrophy (DMD), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD) (Also known as Landouzy-Dejerine), Myotonic Dystrophy (MMD) (Also known as Steinert's Disease), Oculopharyngeal Muscular Dystrophy (OPMD), Distal Muscular Dystrophy (DD), and Congenital Muscular Dystrophy (CMD).

Example motor neuron diseases that can be treated with a composition of this disclosure include: Amyotrophic Lateral Sclerosis (ALS) (Also known as Lou Gehrig's Disease), Infantile Progressive Spinal Muscular Atrophy (SMA, SMA1 or WH) (Also known as SMA Type 1, Werdnig-Hoffman), Intermediate Spinal Muscular Atrophy (SMA or SMA2) (Also known as SMA Type 2), Juvenile Spinal Muscular Atrophy (SMA, SMA3 or KW) (Also known as SMA Type 3, Kugelberg-Welander), Spinal Bulbar Muscular Atrophy (SBMA) (Also known as Kennedys Disease and X-Linked SBMA), and Adult Spinal Muscular Atrophy (SMA).

Example inflammatory myopathies that can be treated with a composition of this disclosure include: Dermatomyositis (PM/DM), Polymyositis (PM/DM), and Inclusion Body Myositis (IBM). Example diseases of the neuromuscular junction that can be treated with a composition of this disclosure include: Myasthenia Gravis (MG), Lambert-Eaton Syndrome (LES), and Congenital Myasthenic Syndrome (CMS). Example myopathies due to endocrine abnormalities that can be treated with a composition of this disclosure include: Hyperthyroid Myopathy (HYPTM) and Hypothyroid Myopathy (HYPOTM). Example diseases of peripheral nerve that can be treated with a composition of this disclosure include: Charcot-Marie-Tooth Disease (CMT), Dejerine-Sottas Disease (DS), and Friedreich's Ataxia (FA). Other example myopathies that can be treated with a composition of this disclosure include: Myotonia Congenita (MC), Paramyotonia Congenita (PC), Central Core Disease (CCD), Nemaline Myopathy (NM), Myotubular Myopathy (MTM or MM), and Periodic Paralysis (PP). Example metabolic diseases of muscle that can be treated with a composition of this disclosure include: Phosphorylase Deficiency (MPD or PYGM), Acid Maltase Deficiency (AMD), Phosphofructokinase Deficiency (PFKM), Debrancher Enzyme Deficiency (DBD), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD).

The antagonist to Fbxo40 of the present disclosure can be used to treat these and various other muscle wasting-associated disorders known in the art.

### Additional Treatments for Muscle Wasting-Associated Disorders

The antagonist of Fbxo40 can be co-administered with another medicament or treatment known or suspected to increase muscle mass or prevent the loss of muscle mass and/or strength. Such treatments include physiotherapy, nutrition, electrical stimulation (e.g., electrical neuromuscular stimulators of NMES), and/or neural input to the muscles.

Various medicaments have been proposed for treating cachexia, sarcopenia and other muscle disorders, including steroids, hormones, including growth hormone, growth hormone secretagogues [including ibutamoren mesylate (MK-677)], gingko biloba extracts (including flavoneglycosides and/or ginkgolides), amino acid supplements (e.g., leucine), amino acid precursors (e.g., leucine precursors such as pyruvate and metabolites, such as beta-hydroxy-beta-methylbutyrate and alpha-ketoisocaproate), branched chain amino acids, erythropoietin, opiates, scopolamine, insulin, insulin-like growth factor-1 (IGF1), and testosterone.

Additional medicaments that can be co-administered with a Fbxo40 antagonist include inhibitors of biological factors (biological agents) and/or genes which are directly or indirectly causative factors of cachexia, or otherwise related to muscle growth. These factors, agents and/or genes include: aldosterone (e.g., spironolactone, testolactone, mespirenone, and canrenoate), alpha receptor (e.g., doxazosin, prazosin, terazosin and ipsapirone), Angiotensin II, beta receptor (acebutolol, alprenolol, atenolol, betaxolol, bisoprolol, carteolol, celiprolol, esmolol, labetolol, lavobunolol, metipranolol, metoprolol, nadolol, oxprenolol, penbutolol, pindolol, propanolol, sotalol, nebivolol, carvedilol, bucindolol and timolol), cathepsin B [e.g., epoxysuccinyl peptides such as CA-074 and E-64c, stefinA, cystatin C (endogenous inhibitor), CA074 (a specific inhibitor of cathepsin B) and E-64 (natural inhibitor of cathepsin B)], chymase [e.g., alendronate, aprotinin and tissue inhibitors of matrix metalloproteinases (TIMPs)], endothelin receptor, eukaryotic initiation factor 2-alpha (eIF2-alpha), imidazoline receptor [e.g., moxonidine, clonidine, rilmenidine, pentamidine (1,5-bis(4-amidonophenoxy)pentane) and alpha methyl dopa], interferon, MAFbx (Muscle Atrophy F-box), MuRF1 (Muscle RING Finger 1), myostatin, parathyroid hormone related protein (PTHrP) and/or its receptor, proteolysis-inducing factor (PIF), RNA-dependent serine/threonine protein kinase (PKR), tumor necrosis factor alpha (TNF-alpha), and xanthine oxidase. In one particular specific embodiment, IGF1 is co-administered with the antagonist to Fbxo40.

For reference, see, for example, U.S. Patent App. 20090105123. See: U.S. Pat. Nos. 6,194,402; 7,232,580; 7,417,038; 7,442,706; and 7,468,184; and United States Patent Applications 20020028838; 20040122097; and 20090105123; and Bodine et al. 2001; McPherron et al. 1997 Proc. Natl. Acad. Sci. 94: 12457-61; Williams 2004 N. Engl. J. Med. 351: 1030-1.

The compositions of the present disclosure can also be used to prevent the loss of muscle mass, or to increase muscle mass in a healthy patient.

In another embodiment of the disclosure, the compositions comprising a Fbxo40 antagonist can be administered to non-human animals. For example, the compositions can be given to chickens, turkeys, livestock animals (such as sheep, pigs, horses, cattle, etc.), companion animals (e.g., cats and dogs) or may have utility in aquaculture to accelerate growth and improve the protein/fat ratio. The compositions can stimulate growth and enhance feed efficiency of animals raised for meat production and improve carcass quality.

### Types of and Efficacy of Antagonists to Fbxo40

As used herein, the term “Fbxo40 antagonist” and the like refer to any moiety, compound, composition or the like which down-regulates Fbxo40 or its activity, level or expression. Such antagonists can comprise, inter alia, low molecular weight compounds (LMWs), antibodies, and/or inhibitory nucleic acids [e.g., short inhibitory RNA (siRNA)]. The antagonist results in a decrease of Fbxo40 activity, level and/or expression, e.g., a “knock-down” or “knock-out” of the gene by targeting the gene, mRNA level and/or protein level.

As used herein, “down-regulates” refers to any statistically significant decrease in a biological activity and/or expression of Fbxo40, including full blocking of the activity (i.e., complete inhibition) and/or expression. For example, “down-regulation” can refer to a decrease of at least about 10, 20, 30, 40, 50, 60, 70, 80, 90 or 100% in Fbxo40 activity and/or expression. The antagonist can, for example, inhibit or degrade Fbxo40 or assist other compounds or biological components in degrading or inhibiting the activity of Fbxo40.

As used herein, the term “inhibit” or inhibiting” Fbxo40 refers to any statistically significant decrease in biological activity and/or expression of Fbxo40, including full blocking of the activity and/or expression. For example, “inhibition” can refer to a decrease of at least about 10, 20, 30, 40, 50, 60, 70, 80, 90 or 100% in Fbxo40 activity and/or expression. As used herein, the term “inhibit” similarly refers to a significant decrease in activity and/or expression, while referring to any other biological agent or composition.

The Fbxo40 antagonist of the present disclosure decreases or down-regulates Fbxo40 expression, level or activity. By “expression,” it is meant that the antagonist can interfere with any of the known biochemical steps involved in expressing a gene, e.g., transcribing DNA into mRNA, processing the mRNA, translating mRNA into protein and post-translationally-modifying the protein. For example, an antagonist that interferes with the expression of Fbxo40 may be involved in preventing the gene from being expressed. This can be performed directly, e.g., by binding to the DNA or mRNA, or indirectly, e.g., by interfering with a transcription factor or transcription co-factor needed to transcribe the gene, or with a factor required for processing the Fbxo40 mRNA.

By “level”, it is meant that the Fbxo40 antagonist can interfere with the detectable level of Fbxo40, e.g., the level of Fbxo40 mRNA or the level of Fbxo40 protein. These levels can be determined by Northern blots, Southern blots, immunoprecipitation, or any of a variety of techniques known in the art.

By “activity,” it is meant that the Fbxo40 antagonist can interfere with any known activity of Fbxo40, as described herein or as known in the literature. In one aspect of the disclosure, the antagonist is any moiety, compound or the like which directly antagonizes Fbxo40, e.g., by preventing or altering the indirect or direct interaction (e.g., binding) of Fbxo40 with another biological component, including, but not limited to Skp1 or IRS1. As a non-limiting example, a Fbxo40 antagonist can be, for example, an antibody which sterically hinders Fbxo40 from binding to Skp1 and/or to IRS1.

An antagonist of Fbxo40 can be, as non-limiting examples, a low-molecular weight composition (LMW), a protein, an antibody, or a short inhibitory RNA (siRNA), or a variant, derivative or fusion thereof.

In various embodiments of the present disclosure, the Fbxo40 antagonist (including, but not limited to, a LMW, protein, or antibody) can interact with any of the known or putative structures of Fbxo40. These Fbxo40 structures include, but are not limited to, the F-box motif at approximately aa (amino acids) 570-624 and the zinc finger TRAF-type domain at aa 54-96 (as described in Ye et al. 2007). In another embodiment, the Fbxo40 antagonist is an antibody which does not bind in the region from aa 145-372; thus the Fbxo40 antagonist is an antibody which binds in the region from aa 1-143 or 373-709.

In additional embodiments of the present disclosure, the Fbxo40 antagonist interacts with an amino acid of Fbxo40 which is conserved relative to other members of the Fbox family, including the F-box sequence. The consensus sequence for this Fbox motif is provided in Kipreos et al. 2000 Genome Biol. 1(5): REVIEWS3002. The Fbox motif from Fbxo40 lies at approximately aa 570 to 624. Ye et al. 2007.

In various embodiments, the present disclosure provides the following provisos: the Fbxo40 antagonist interacts with (e.g., physically binds to) Fbxo40 but not at any one or more specific structure or sequence listed; thus, the Fbxo40 antagonist in various embodiments can interact with the Fbxo40 gene or protein but not at the Fbox motif at aa 570 to 624; or the Fbxo40 antagonist in various embodiments can interact with the Fbxo40 gene or protein but not at the zinc finger domain from aa 54 to 96.

In one embodiment, the present disclosure has the proviso that the Fbxo40 antagonist is not a polyclonal antibody. In another embodiment, the present disclosure has the proviso that the Fbxo40 antagonist is not a polyclonal antibody that is raised against or binds to the Fbxo40 sequence CEKARESLVSTFRARPRGRHF (SEQ ID NO: 34).

In one embodiment of the present disclosure, the Fbxo antagonist is a siRNA that targets the sequence of CACCTCCTGGAAAGTCCACAA (SEQ ID NO: 19), GTGGGAAAGTATGTTCAGCAA (SEQ ID NO: 20) or AGCCGTGGATGCCAAAGACTA (SEQ ID NO: 21) (or the RNA equivalent).

Administration of the antagonist to Fbxo40 results in muscle hypertrophy, or the prevention, limitation or reduction of muscle loss.

By “muscle hypertrophy”, “muscle growth” and the like is meant an increase in muscle mass. This can include an increase in the size, rather than number, of muscle fibers. These muscle fibers can include heart and skeletal muscle, including weight-bearing and non-weight-bearing muscles. Muscle hypertrophy can be measured by various methods known in the art, including measuring the average cross-sectional areas of individual muscle fibers. Muscle hypertrophy can be measured in vitro (e.g., with C2C12 myotubes) or in vivo.

By “prevention, limitation or reduction of muscle loss” and similar phrases is meant that administration of the Fbxo40 antagonist prevents, limits or reduces the amount or rate of muscle loss usually associated with a particular condition, such as cachexia or anorexia.

As non-limiting particular specific examples, the antagonist to Fbxo40 can comprise a low molecular weight composition (or compound), an antibody or the like, and/or and inhibitory nucleic acid or siRNA or the like.

### Low Molecular Weight Compositions as Antagonists to Fbxo40

An antagonist of Fbxo40 can be a low molecular weight composition (LMW) or small molecule. In one embodiment, the Fbxo40 antagonists employed in the methods of the disclosure are small molecules. As used herein, the term “small molecule” is a term of the art and includes molecules that are less than about 7500, 7000, 6000, 5000, 4000, 3000, 2500, 2000, 1500, 1000, 900, 800, 700, 600, 500, 400, 300, 200, or 100 molecular weight, and inhibit Fbxo40 activity. Example small molecules include, but are not limited to, small organic molecules (e.g., Cane et al. 1998. Science 282: 63), and natural product extract libraries. In another embodiment, the compounds are small, organic non-peptidic compounds. Like antibodies, these small molecule inhibitors indirectly or directly inhibit the activity of Fbxo40.

In another embodiment of the disclosure, the Fbxo40 antagonist is a protein.

In one particular specific embodiment, the present disclosure encompasses a method of screening compositions for the ability to increase muscle mass or prevent the loss of muscle mass in an individual, comprising:
ascertaining the level or activity of Fbxo40 in a cell,
treating the cell with a composition, and
ascertaining the level or activity of Fbxo40 in the cell again,
   wherein an ability of the composition to decrease the level or activity of Fbxo40 is correlated to the ability to increase muscle mass or prevent the loss of muscle mass in an individual.

To obtain LMWs that inhibit Fbxo40, a library of compounds can be created including numerous variants of a compound. Library compounds are tested for specific inhibition of a biological activity of Fbxo40 (e.g., binding to IRS1 or Skp1). Selected compounds can be used as the basis for further randomization and selection to produce derivatives of higher affinity or inhibitory activity.

Methods for developing libraries of LMWs and methods of screening them for binding to a target protein are known in the art. For example, the U.S. Pat. No. 7,377,894 describes a method for building a library of up to 10,000 compounds, a majority of which are preferably no more than 350 grams/mole. This technique involves selecting compounds having a solubility in deuterated water of at least about 1 mM at room temperature, and uses flow nuclear magnetic resonance (NMR) spectroscopy and water-ligand observation with gradient spectroscopy (“WaterLOGSY”) methods. This method involves identifying a compound that binds to a target molecule (e.g., a protein), based on NMR spectroscopy techniques. Such methods typically involve the use of relaxation-editing techniques, for example, which involve monitoring changes in resonance intensities (preferably, significant reductions in intensities) of the test compound upon the addition of a target molecule. Preferably, the relaxation-editing techniques are one-dimensional, and more preferably, one-dimensional ¹H NMR techniques. Alternatively, such methods can involve the use of WaterLOGSY. This involves the transfer of magnetization from bulk water to detect the binding interaction. Using WaterLOGSY techniques, binding compounds are distinguished from non-binders by the opposite sign of their water-ligand nuclear Overhauser effects (NOEs).

Other techniques for developing libraries of compounds are known. U.S. Pat. No. 7,367,933 describes a method of producing a chemical compound library comprises extracting at least one extract from at least one species of plant.

Any of these methods, or other methods known in the art, can be used to produce libraries of LMWs, which can be screened for binding and antagonizing Fbxo40.

Methods of screening libraries of compounds for binding to a target (in this case, Fbxo40) are known in the art.

U.S. Pat. No. 7,238,490 is related to real-time detection of intermolecular interaction and states that intermolecular binding can be detected by formation of a “paratope” which results in an immediate generation of a signal. The substances to be tested for interaction are bound to demitopes, wherein said demitopes are components of a paratope which binds a reporter which provides said signal when bound. Known interactions measured in this way can also be employed to screen for compounds which interfere with the interactions. In addition to testing for individual interactions, the interaction of a compound with a library or library.times.library interactions can also be determined and the effect of potentially interfering substances evaluated.

Various other methods for creating and screening compound libraries are described in, inter alia, U.S. Pat. Nos. 6,764,858; 6,723,235; 6,720,190; 6,677,160; 6,656,739; 6,649,415; 6,630,835; 6,627,453; 6,617,114; 6,613,575; 6,607,921; 6,602,685; 6,448,794; 6,421,612; 6,395,169; 6,387,257; 6,355,163; 6,214,561; 6,187,923; and 6,054,047.

Any of these methods for creating and screening libraries of LMWs or any other method known to one of ordinary skill in the art can be used to obtain a small molecule that binds to and antagonized Fbxo40.

### Antibodies and the Like as Antagonists to Fbxo40

An antagonist of Fbxo40 can also be an anti-Fbxo40 antibody, antibody-like molecule, and/or molecule which binds specifically and/or selectively to Fbxo40, or variant, derivative or immunoconjugate thereof, and the like.

In one embodiment of the disclosure, the therapeutic and diagnostic methods described herein employ an antibody or immunoglobulin that binds to (directly or indirectly) and inhibits Fbxo40 activity by interrupting the binding of Fbxo40 to Skp1-Cullin1-Rbx1 complex and/or down-modulates Fbxo40 expression (a neutralizing antibody).

The terms “antibody” or “immunoglobulin” and the like include any whole antibody, any antigen-binding portion, any one or more CDR region(s), fragment, or single chain thereof, and molecules which mimic binding affinities and antigen-binding portions of antibodies, and variants and derivatives thereof. An “antibody” comprises two heavy (H) chains and two light (L) chains connected by disulfide bonds. Each heavy chain comprises a heavy chain variable region (V_{H}) and a heavy chain constant region, the latter comprising three domains, CH1, CH2 and CH3. Each light chain comprises a light chain variable region (V_{L}) and a light chain constant region, comprising one domain, CL. The V_{H} and V_{L} regions can be further subdivided into regions of hypervariability [complementarity determining regions (CDR)], interspersed with regions that are more conserved [framework regions (FR)]. Each V_{H} and V_{L} is composed of three CDRs and four FRs. The variable regions of the heavy and light chains contain a binding domain that interacts with an antigen. The constant regions may mediate the binding of the antibody to host tissues or factors, including cells of the immune system (e.g., effector cells) and the first component (Clq) of the classical complement system.

An anti-Fbxo40 antibody of the disclosure includes, but is not limited to, any derivative or variant of an antibody, an antibody-like molecule, an antigen-binding portion of an antibody, and any monoclonal, polyclonal, recombinant, chimeric, human, non-human, humanized, bispecific, bifunctional, isotype-switched, non-isotype-switched antibody (or variant or derivative thereof) which binds to Fbxo40. The antibody to Fbxo40 is preferably monoclonal. The anti-Fbxo40 antibody of the present disclosure also includes camelid nanobodies, diabodies, single-chain diabodies, and di-diabodies which bind to and antagonize Fbxo40.

The present disclosure also encompasses sets of two or more antibodies, variants or antibody-like molecules which can non-competitively bind to Fbxo40. Preferably, the affinity of the set or combination of molecules is higher than that of any of the constituent molecules.

The term “antigen-binding portion” of an antibody, as used herein, refers to a fragment(s) of an antibody that retains the ability to specifically bind to an antigen (e.g., Fbxo40). Examples of binding fragments include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including VH and VL domains; (vi) a dAb fragment [Ward et al. 1989 Nature 341, 544-546], which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. These compositions, inter alia, are also encompassed by the term “antibody-like molecule.” The two domains of the Fv fragment, V_{L} and V_{H}, are coded for by separate genes, but they can be joined, using recombinant methods, by a synthetic linker, creating a monovalent single protein chain known as single chain Fv (scFv). Bird et al. 1988 Science 242, 423-426; and Huston et al. 1988 Proc. Natl. Acad. Sci. USA 85, 5879-5883. Antigen-binding portions can be produced by recombinant DNA techniques, or by enzymatic or chemical cleavage of intact immunoglobulins.

The term “monoclonal antibody” as used herein refers to an antibody from a population of substantially homogeneous antibodies, e.g., which bind to and antagonize Fbxo40. Monoclonal antibodies can be prepared, e.g., using various methods, e.g., Kohler et al. 1975 Nature, 256: 495; Lonberg, et al. 1994 Nature 368: 856-859; U.S. Pat. No. 4,816,567; Clackson et al. 1991 Nature, 352: 624-628 and Marks et al. 1991 J. Mol. Biol., 222: 581-597.

In contrast to polyclonal antibody preparations, which include different antibodies to different epitopes, each monoclonal antibody is directed against a single determinant on the antigen. Monoclonal antibodies are thus highly specific, directed against a single antigenic site or epitope.

The term “epitope” or “antigenic determinant” refers to a site on an antigen, e.g., Fbxo40, to which an antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least about 3, 4, 5, 6, 7, 8, 9, 10, 11, 12, 13, 14 or 15 amino acids in a unique spatial conformation. Methods of determining spatial conformation of epitopes include techniques in the art and those described herein, e.g., X-ray crystallography and 2-dimensional nuclear magnetic resonance. See, e.g., Epitope Mapping Protocols in Methods in Molecular Biology, Vol. 66, G. E. Morris, Ed. 1996.

Monoclonal antibodies include chimeric antibodies, human antibodies and humanized antibodies and may occur naturally or be recombinantly produced.

The term “recombinant antibody” refers to antibodies that are prepared, expressed, created or isolated by recombinant means, such as (a) antibodies isolated from an animal (e.g., a mouse) that is transgenic or transchromosomal for immunoglobulin genes (e.g., human genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human genes) to other DNA sequences. Such recombinant antibodies may have variable and constant regions derived from human germline immunoglobulin sequences. Such recombinant human antibodies can be subjected to in vitro mutagenesis and thus the amino acid sequences of the V_{H} and V_{L} regions of the recombinant antibodies are sequences that, while derived from human germline sequences, may not naturally exist within the human antibody germline repertoire in vivo.

The term “chimeric antibody” refers to an immunoglobulin or antibody whose variable regions derive from a first species and whose constant regions derive from a second species.

The term “human antibody,” as used herein, is intended to include antibodies having variable regions in which both the framework and CDRs are derived from human germline immunoglobulin sequences. Kabat, et al. (1991) Sequences of Proteins of Immunological Interest, Fifth Edition, U.S. Department of Health and Human Services, NIH Publication No. 91-3242. CDR and framework consensus sequences have also been described in Chothia et al., J. Mol. Biol. 196:901-917 (1987) and by MacCallum et al., J. Mol. Biol. 262:732-745 (1996); and Chothia et al., 1998, J. Mol. Biol. 278: 457-479. Any of these, but preferably Kabat or Chothia, can be used to determine the CDR sequences within a particular antibody variable region.

A human antibody can include a variant of a wildtype human germline sequence.

The term “humanized antibody” refers to an antibody that includes at least one humanized light or heavy chain, which has a variable region substantially from a human antibody and CDRs substantially from a non-human antibody, along with constant regions. The term “humanized variable region” refers to a variable region that includes a variable framework region substantially from a human antibody and CDRs substantially from a non-human antibody.

The term “bi-specific” or “bi-functional antibody” includes, inter alia, an artificial hybrid antibody having two different heavy/light chain pairs and two different binding sites. Songsivilai et al. 1990 Clin. Exp. Immunol. 79: 315-321; Kostelny et al. 1992 J. Immunol. 148: 1547-1553.

As used herein, a “heterologous antibody” is defined in relation to the transgenic non-human organism or plant producing such an antibody.

The antibodies of the present disclosure encompass, inter alia, isotype-switched and non-isotype-switched antibodies.

As used herein, “isotype” refers to the antibody class (e.g., IgM or IgG, etc.) that is encoded by heavy chain constant region genes. In one embodiment, an antibody or antigen binding portion thereof is of an isotype selected from an IgG1, an IgG2, an IgG3, an IgG4, an IgM, an IgA1, an IgA2, an IgAsec, an IgD, or an IgE antibody isotype.

As used herein, “isotype switching” refers to the phenomenon by which the class, or isotype, of an antibody changes from one Ig class to one of the other Ig classes.

As used herein, “non-switched isotype” refers to the isotypic class of heavy chain that is produced when no isotype switching has taken place; the CH gene encoding the non-switched isotype is typically the first CH gene immediately downstream from the functionally rearranged VDJ gene.

The anti-Fbxo40 antibody of the present disclosure also includes camelid nanobodies, diabodies, single-chain diabodies, and di-diabodies which bind to and antagonize Fbxo40.

Antibody proteins obtained from members of the camel and dromedary (Camelus bactrianus and Calelus dromaderius) family, including New World members such as llama species (Lama paccos, Lama glama and Lama vicugna), have been characterized. Certain IgG antibodies found in these mammals lack light chains, and are thus structurally distinct from the antibodies from other animals. PCT Publication WO 94/04678. The small, single variable domain (V_{HH}) of the camelid antibody yields a high affinity, low molecular weight, antibody-derived protein known as a “camelid nanobody”. U.S. Pat. No. 5,759,808; Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, e.g., from Ablynx, Ghent, Belgium. The nanobody can be “humanized” and the natural low antigenicity of camelid antibodies can be further reduced.

The camelid nanobody has a molecular weight approximately one-tenth that of a human IgG molecule, and has a diameter of only a few nanometers. Camelid nanobodies can bind to antigenic sites functionally invisible to larger antibody proteins.

Camelid nanobodies are thermostable, stable to extreme pH and to proteolytic digestion, and poorly antigenic. They readily move from the circulatory system into tissues, and even cross the blood-brain barrier and can treat disorders that affect nervous tissue. Nanobodies can further facilitate drug transport across the blood brain barrier. U.S. Pat. Pub. No. 20040161738, published Aug. 19, 2004.

The antibodies, antibody-like molecules and other molecules which bind specifically and/or selectively to Fbxo40 of the present disclosure also include, inter alia, diabodies, single chain diabodies (scDb), molecules that exhibit functional properties of antibodies but derive their framework and antigen binding portions from other polypeptides (e.g., fibronectins and fibronectin-like molecules), and any and all antibody fragments and mimetics, including, but not limited to, e.g., domain antibodies, Nanobodies, UniBodies, Adnectins, aptamers, Affibodies, DARPins, Anticalins, Avimers, Versabodies, and/or SMIPs™ (Small Modular ImmunoPharmaceuticals-Trubion Pharmaceuticals).

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites. Holliger et al., 1993 Proc. Natl. Acad. Sci. USA 90: 6444-6448; Poljak et al., 1994 Structure 2: 1121-1123.

Single chain diabodies (scDb) are produced by connecting the two diabody-forming polypeptide chains with linker of approximately 15 amino acid residues. Holliger et al. 1997 Cancer Immunol. Immunother., 45: 128-30; Wu et al., 1996 Immunotechnology, 2: 21-36; Pluckthun et al. 1997 Immunotech. 3: 83-105; Ridgway et al., 1996 Protein Eng., 9: 617-21. A diabody can be fused to Fc to generate a “di-diabody”. Lu et al., 2004 J. Biol. Chem., 279: 2856-65.

The disclosure further provides Fbxo40-binding molecules that exhibit functional properties of antibodies but derive their framework and antigen binding portions from other polypeptides. The antigen binding domains of these binding molecules can be generated through a directed evolution process. U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1,1-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The terms “Fbxo40 antibody”, “Fbxo40 antibody-like molecule,” “molecule which binds specifically and/or selectively to Fbxo40” and the like also broadly include, but is not limited to, antibody fragments and antibody mimetics. A wide variety of antibody fragment and antibody mimetic technologies are known. The terms “Fbxo40 antibody”, “Fbxo40 antibody-like molecule” and “molecule which binds specifically and/or selectively to Fbxo40” are broadly meant to encompass any and all antibody fragments and mimetics, including, but not limited to, e.g., Domain Antibodies, Nanobodies, UniBodies, Adnectins, aptamers, Affibodies, DARPins, Anticalins, Avimers, and Versabodies. Some of these molecules are reviewed in Gill and Damle (2006) 17: 653-658.

Domain Antibodies (dAbs) are the smallest functional binding units of antibodies, corresponding to the variable regions of either the heavy (VH) or light (VL) chains of human antibodies. Domantis has developed a series of large and highly functional libraries of fully human VH and VL dAbs, and uses these libraries to select dAbs that are specific to therapeutic targets. U.S. Pat. Nos. 6,291,158; 6,582,915; 6,593,081; 6,172,197; 6,696,245; U.S. Serial No. 2004/0110941; European patent application No. 1433846 and European Patents 0368684 & 0616640; WO05/035572, WO04/101790, WO04/081026, WO04/058821, WO04/003019 and WO03/002609.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). WO 04/041867; U.S. Pat. No. 6,765,087; WO 06/079372.

UniBodies are a antibody fragment technology based upon the removal of the hinge region of IgG4 antibodies. This deletion produces a molecule that is essentially half the size of traditional IgG4 antibodies and has a univalent binding region. WO2007/059782.

Adnectin molecules are engineered binding proteins derived from one or more domains of the fibronectin protein. Ward et al., callutheran.edu/Academic_Programs/Departments/BioDev/omm/fibro/fibro.htm; Pankov and Yamada (2002) J Cell Sci. 115 (Pt 20): 3861-3, Hohenester and Engel (2002) 21: 115-128, and Lucena et al. (2007) Invest Clin. 48: 249-262. Adnectin molecules can be derived from the fibronectin type III domain by altering the native protein, which is composed of multiple beta strands distributed between two beta sheets.

The present disclosure also broadly encompasses and fibronectin or fibronectin-like molecule which specifically and/or selectively binds to Fbxo40. Fibronectins may contain multiple type III domains which may be denoted, e.g., ¹Fn3, ²Fn3, ³Fn3, etc. The ¹⁰Fn3 domain contains an integrin binding motif and further contains three loops which connect the beta strands. These loops may be thought of as corresponding to the antigen binding loops of the IgG heavy chain, and they may be altered by methods discussed below to specifically bind Fbxo40. U.S. Pat. Application No. 20070082365; Szostak et al., U.S. Ser. No. 09/007,005 and 09/247,190; Szostak et al., WO989/31700; and Roberts & Szostak (1997) 94: 12297-12302; Lohse, U.S. Ser. No. 60/110,549, U.S. Ser. No. 09/459,190, and WO 00/32823; U.S. Pat. Nos. 7,115,396; 6,818,418; 6,537,749; 6,660,473; 7,195,880; 6,416,950; 6,214,553; 6,623,926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. Pat. Application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. The generation of diversity in fibronectin type III domains, such as ¹⁰Fn3, followed by a selection step may be accomplished using other methods known in the art such as phage display, ribosome display, or yeast surface display, e.g., Lipov\check{s}ek et al. (2007) Journal of Molecular Biology 368: 1024-1041; Sergeeva et al. (2006) Adv Drug Deliv Rev. 58: 1622-1654; Petty et al. (2007) Trends Biotechnol. 25: 7-15; Rothe et al. (2006) Expert Opin Biol Ther. 6: 177-187; and Hoogenboom (2005) Nat. Biotechnol. 23: 1105-1116.

Additional molecules which can be used to generate antibody mimics via the above referenced methods include, without limitation, human fibronectin modules ¹Fn3⁻⁹Fn3 and ¹¹Fn3-¹⁷Fn3 as well as related Fn3 modules from non-human animals and prokaryotes, and Fn3 modules from other proteins with sequence homology to ¹⁰Fn3, such as tenascins and undulins. Other non-antibody proteins having immunoglobulin-like folds include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule P0, CD8, CD4, CD2, class I MHC, T-cell antigen receptor, CD1, C2 and I-set domains of VCAM-1,1-set immunoglobulin fold of myosin-binding protein C, I-set immunoglobulin fold of myosin-binding protein H, I-set immunoglobulin-fold of telokin, telikin, NCAM, twitchin, neuroglian, growth hormone receptor, erythropoietin receptor, prolactin receptor, GC-SF receptor, interferon-gamma receptor, beta-galactosidase/glucuronidase, beta-glucuronidase, and transglutaminase. Alternatively, any other protein that includes one or more immunoglobulin-like folds may be utilized to create an adnectin-like binding moiety. Such proteins may be identified, e.g., using the program SCOP. Murzin et al., J. Mol. Biol. 247: 536 (1995); Lo Conte et al., Nucleic Acids Res. 25: 257 (2000).

An aptamer is a small nucleotide polymer that binds to specific targets. Aptamers may be single or double stranded nucleic acid molecules (DNA or RNA). Aptamers often form complex three-dimensional structures which determine their affinity for targets. Ellington et al. 1990 Nature. 346: 818-22; Schneider et al. 1992. J Mol. Biol. 228: 862-9; Klussmann. The Aptamer Handbook Functional Oligonucleotides and Their Applications. ISBN: 978-3-527-31059-3; Ulrich et al. 2006. Comb Chem High Throughput Screen 9: 619-32; Cerchia et al. 2007. Methods Mol Biol. 361: 187-200; Ireson et al. 2006. Mol Cancer Ther. 2006 5: 2957-62; U.S. Pat. Nos. 5,582,981; 5,840,867; 5,756,291; 6,261,783; 6,458,559; 5,792,613; 6,111,095; and U.S. patent application Ser. Nos. 11/482,671; 11/102,428; 11/291,610; and 10/627,543. The SELEX method can be used to generate aptamers. Bugaut et al. 2006. 4(22): 4082-8; Stoltenburg et al. 2007 Biomol. Eng. 2007 24(4): 381-403; and Gopinath. 2007. Anal. Bioanal Chem. 2007. 387(1): 171-82. An aptamer of the disclosure also been includes aptamer molecules made from peptides instead of nucleotides. Baines and Colas. 2006. Drug Discov. Today. 11(7-8): 334-41; and Bickle et al. 2006. Nat. Protoc. 1(3): 1066-91.

Affibody molecules are based on a 58-amino acid residue protein domain, derived from a IgG-binding domain of staphylococcal protein A. This domain has been used as a scaffold for the construction of combinatorial phagemid libraries, from which variants that target the desired molecules can be selected using phage display technology (Nord et al. Nat. Biotechnol 1997; 15: 772-7; Ronmark et al., Eur J Biochem 2002; 269: 2647-55). The simple structure and small size of Affibody molecules make them suitable for many applications, e.g., as detection reagents (Ronmark et al., J Immunol Methods 2002; 261: 199-211) and to inhibit receptor interactions (Sandstorm et al. Protein Eng 2003; 16: 691-7). See also, U.S. Pat. No. 5,831,012.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules with repeating structural units, which stack together to form elongated domains displaying variable and modular target-binding surfaces. Combinatorial libraries of polypeptides with diversified binding specificities can be generated. U.S. Patent Application Publication No. 2004/0132028 and International Patent Application Publication No. WO 02/20565.

Anticalins are antibody mimetics derived from lipocalins, a family of low molecular weight proteins expressed in human tissues and body fluids. Lipocalins are associated with the physiological transport and storage of chemically sensitive or insoluble compounds. Lipocalins are cloned and their loops are subjected to engineering in order to create Anticalins. Libraries of structurally diverse Anticalins have been generated and Anticalin display allows the selection and screening of binding function, followed by the expression and production of soluble protein. Anticalins can also be formatted as dual targeting proteins, so-called Duocalins. A Duocalin binds two separate therapeutic targets in one monomeric protein. U.S. Pat. No. 7,250,297 and International Patent Application Publication No. WO 99/16873.

Another antibody mimetic technology useful for the instant disclosure are Avimers. Avimers are evolved from a large family of human extracellular receptor domains by in vitro exon shuffling and phage display, generating multidomain proteins with binding and inhibitory properties. U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756.

Versabodies are another antibody mimetic technology that could be used in the context of the instant disclosure. Versabodies are small proteins of 3-5 kDa with >15% cysteines, which form a high disulfide density scaffold, replacing the hydrophobic core that typical proteins have. U.S. Patent Application Publication No. 2007/0191272.

SMIPs™ (Small Modular ImmunoPharmaceuticals-Trubion Pharmaceuticals) are engineered to maintain and optimize target binding, effector functions, in vivo half life, and expression levels. Zhao et al. (2007) Blood 110: 2569-77; and U.S. Pat. App. Nos. 20050238646; 20050202534; 20050202028; 20050202023; 20050202012; 20050186216; 20050180970; and 20050175614.

A Fbxo40 antibody (or antibody-like molecule, or molecule which specifically and/or selectively binds to Fbxo40, or variant or derivative thereof, and the like) of the present disclosure also retains specific binding to Fbxo40, and/or similar K_{D}, K_{off}, and/or EC50 of a Fbxo40 antibody. This variant, derivative or antibody-like molecule can optionally have the same or different glycosylation pattern, or be naturally-occurring or re-arranged, can have modifications, conservative or non-conservative substitutions, and/or retain the consensus framework of a Fbxo40 antibody.

Accordingly, as used herein, the terms “antibody to Fbxo40,” “Fbxo40 antibody,” “antibody-like molecule” which binds Fbxo40, and the like all refer to the various types of antibodies, antibody fragments, variants and derivatives, antibody-like molecules, and molecules which bind with specificity, which specifically and/or selectively bind to Fbxo40. Preferably these compositions also inhibit a function of Fbxo40, e.g., a function described herein.

As used herein, the terms “specific binding,” “selective binding,” and the like mean that an antibody or other molecule exhibits appreciable affinity for a particular antigen or epitope but not other antigens and epitopes, e.g., Fbxo40 but not other entities (unless the antibody or molecule also binds to Fbxo40). “Appreciable” or particular specific binding includes binding with an affinity of at least 10⁶, 10⁷, 10⁸, 10⁹ M⁻¹, or 10¹⁰ M⁻¹. Affinities greater than 10⁷M⁻¹, preferably greater than 10⁸ M⁻¹ are more preferred. An antibody that “does not exhibit significant cross-reactivity” is one that will not appreciably bind to an undesirable entity (e.g., an undesirable proteinaceous entity). Specific or selective binding can be determined according to any art-recognized means, including, e.g., according to Scatchard analysis and/or competitive binding assays.

The term “K_{D},” as used herein, is intended to refer to the dissociation equilibrium constant of a particular antibody-antigen interaction or the affinity of an antibody for an antigen. In one embodiment, the antibody according to the present disclosure binds an antigen with an affinity (K_{D}) of 50 nM or better (e.g., 40 nM or 30 nM or 20 nM or 10 nM or less), as measured using a surface plasmon resonance assay or a cell binding assay.

The term “K_{off},” as used herein, is intended to refer to the off rate constant for the dissociation of an antibody from the antibody/antigen complex.

The term “EC50,” as used herein, refers to the concentration of an antibody which induces a response, either in an in vitro or an in vivo assay, which is 50% of the maximal response, i.e., halfway between the maximal response and the baseline.

The antibodies, antibody-like molecules and other molecules that specifically and/or selectively bind Fbxo40 of the present disclosure thus include, without limitation, inter alia, any of the molecule types listed herein, and various other molecules which specifically bind, as are known in the art. These molecules can be generated using any technique known in the art, including, but not limited to, those listed herein.

Technologies for generating these molecules include alternative polypeptide-based technologies, such as fusions of complimentary determining regions as outlined in Qui et al., Nature Biotechnology, 25(8) 921-929 (2007), as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620.

To generate non-antibody binding molecules, a library of clones can be created in which sequences of a scaffold protein (e.g., a fibronectin or fibronectin-like molecule) that bind the antigen are randomized. Library clones are tested for specific binding to the antigen and for other functions (e.g., inhibition of a biological activity of Fbxo40). Selected clones can be used as the basis for further randomization and selection to produce derivatives of higher affinity for the antigen.

High-affinity binding molecules can also be generated, e.g., using the tenth module of fibronectin III (¹⁰Fn3 or Fn10) as the scaffold. A library is constructed for each of three CDR-like loops of ¹⁰FN3 at residues 23-29, 52-55, and 78-87. U.S. Pat. Nos. 6,818,418 and 7,115,396; Roberts and Szostak, 1997 Proc. Natl. Acad. Sci. USA 94: 12297; U.S. Pat. No. 6,261,804; U.S. Pat. No. 6,258,558; and Szostak et al. WO98/31700.

Non-antibody binding molecules can be produced as dimers or multimers to increase avidity for the target. For example, the antigen binding domain is expressed as a fusion with a constant region (Fc) of an antibody that forms Fc-Fc dimers. U.S. Pat. No. 7,115,396.

Antibodies that recognize the same or an overlapping epitope can be identified using routine techniques such as an immunoassay, e.g., a competitive binding assay. Numerous types of competitive binding assays are known, e.g.: solid phase direct or indirect radioimmunoassay (RIA), solid phase direct or indirect enzyme immunoassay (EIA), sandwich competition assay (see Stahli et al., (1983) Methods in Enzymology 9: 242); solid phase direct biotin-avidin EIA (see Kirkland et al., (1986) J. Immunol. 137: 3614); solid phase direct labeled assay, solid phase direct labeled sandwich assay (see Harlow and Lane, (1988) Antibodies: A Laboratory Manual, Cold Spring Harbor Press); solid phase direct label RIA using I-125 label (see Morel et al., (1988) Mol. Immunol. 25(1): 7); solid phase direct biotin-avidin EIA (Cheung et al., (1990) Virology 176: 546); and direct labeled RIA. (Moldenhauer et al., (1990) Scand. J. Immunol. 32: 77).

The antibodies, antibody-like molecules, and other binding molecules which specifically bind Fbxo40 can also be modified. These modifications include, inter alia, changes from the state of the molecule as found in nature (the “naturally-occurring” state), changes in the glycosylation pattern, rearrangement, modifications of amino acid sequence (including, inter alia, conservative and non-conservative substitutions), CDR grafting, affinity maturation, modification of the Fc region or hinge region, and/or change in pegylation or other post-translational modification, and the like.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein, “glycosylation pattern” is defined as the pattern of carbohydrate units that are covalently attached to a protein, more specifically to an immunoglobulin protein.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

The term “modifying,” or “modification,” as used herein, is intended to refer to changing one or more amino acids in the antibodies. The change can be produced by adding, substituting or deleting an amino acid at one or more positions. The antibodies, antibody-like molecules and other molecules which bind specifically and/or selectively to Fbxo40 can have modifications including conservative and non-conservative amino acid substitutions.

The present disclosure thus encompasses “conservative amino acid substitutions” in that nucleotide and amino acid sequence modifications that do not abrogate the binding of the antibody to Fbxo40. Conservative amino acid substitutions include the substitution of an amino acid in one class by an amino acid of the same class. Six general classes of amino acid side chains include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). Brummell et al., Biochem. 32: 1180-1187 (1993); Kobayashi et al. Protein Eng. 12(10): 879-884 (1999); and Burks et al. Proc. Natl. Acad. Sci. USA 94: 0.412-417 (1997).

The term “non-conservative amino acid substitution” refers to the substitution of an amino acid in one class with an amino acid from another class.

Alternatively, in another embodiment, mutations (conservative or non-conservative) can be introduced randomly along all or part of an antibody coding sequence, such as by saturation mutagenesis, and the resulting modified antibodies can be screened for binding activity. The conservative and non-conservative modifications can occur in a consensus sequence of an antibody, antibody-like molecule or other molecule which binds specifically and/or selectively to Fbxo40.

A “consensus sequence” is a sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” of an immunoglobulin refers to a framework region in the consensus immunoglobulin sequence. Other consensus sequences of antibodies, antibody-like molecules and other molecules which bind specifically and/or selectively are known in the art.

Additional modified versions of these compositions can be prepared using techniques known to a person of ordinary skill in the art. An antibody of the disclosure can be prepared using an antibody having one or more V_{H} and/or V_{L} sequences as starting material to engineer a modified antibody, which can have altered properties from the starting antibody. An antibody can be engineered by modifying one or more residues within one or both variable regions. A Fbxo40 antibody of the present disclosure can have one or more CDR graft, mutated amino acid, affinity matured sequence, and/or modification of the Fc region, hinge region, glycosylation pattern and/or pegylation pattern.

One type of variable region engineering that can be performed is CDR grafting; CDR sequences from one antibody are grafted onto framework sequences from a different antibody with different properties. Riechmann et al., 1998 Nature 332: 323-327; Jones et al., 1986 Nature 321: 522-525; Queen et al., 1989 Proc. Natl. Acad. See. U.S.A. 86: 10029-10033; U.S. Pat. No. 5,225,539, and U.S. Pat. Nos. 5,530,101; 5,585,089; 5,693,762 and 6,180,370). Methods of CDR grafting and appropriate sequences are known. For example, see www.mrc-cpe.cam.ac.uk/vbase; Kabat et al., 1991 Sequences of Proteins of Immunological Interest, Fifth Edition, U.S. Dept. of Health and Human Services, NIH Publication No. 91-3242; Tomlinson et al., 1992 J. Mol. Biol. 227: 776-798; and Cox et al., 1994 Eur. J. Immunol. 24: 827-836; U.S. Pat. Nos. 5,530,101; 5,585,089; 5,693,762 and 6,180,370). CDRs can also be grafted into framework regions of polypeptides other than immunoglobulin domains, e.g., a framework based on fibronectin, ankyrin, lipocalin, neocarzinostain, cytochrome b, CP1 zinc finger, PST1, coiled coil, LACI-D1, Z domain or tendramisat. Nygren and Uhlen, 1997 Current Opinion in Structural Biology, 7, 463-469).

Amino acid residue(s) within the V_{H} and/or V_{L} CDR1, CDR2 and/or CDR3 regions can be mutated to improve binding properties of the antibody, known as “affinity maturation.” The mutations can be amino acid substitutions, additions, deletions, conservative or non-conservative changes, and/or use of a chemically-modified amino acid. Modifications can be made to framework within V_{H} and/or V_{L}, e.g., to improve the properties of the antibody, e.g., to decrease immunogenicity. One approach is to “backmutate” one or more framework residues to the corresponding germline sequence. More specifically, an antibody that has undergone somatic mutation may contain framework residues that differ from the germline sequence from which the antibody is derived. The somatic mutations can be “backmutated” to the germline sequence.

Another type of framework modification involves mutating one or more residues to remove T cell-epitopes to reduce the potential immunogenicity (“deimmunize”) of the antibody. U.S. Pat. Pub. No. 20030153043 by Carr et al.

Antibodies of the disclosure can be modified within the Fc region, e.g., to alter one or more properties, e.g., serum half-life, complement fixation, Fc receptor binding, and/or antigen-dependent cellular cytotoxicity. Furthermore, an antibody of the disclosure may be chemically modified (e.g., one or more chemical moieties can be attached to the antibody) or be modified to alter its glycosylation, again to alter one or more functional properties of the antibody.

In one embodiment, the hinge region of CH1 is modified such that the number of cysteine residues in the hinge region is altered. U.S. Pat. No. 5,677,425. The Fc hinge region can also be mutated to alter the biological half-life of the antibody. U.S. Pat. No. 6,165,745.

The antibody can be modified to increase its biological half-life. U.S. Pat. Nos. 6,277,375; 5,869,046 and 6,121,022.

In various embodiments of the antibody of the disclosure, one of more amino acids can be modified (substituted, added, deleted, chemically changed, or conservatively substituted) to alter the effector functions, e.g., affinity for an effector ligand (e.g., an Fc receptor or the C1 component of complement) and/or the antigen-binding ability, U.S. Pat. Nos. 5,624,821 and 5,648,260; to alter Clq binding and/or complement dependent cytotoxicity (CDC), U.S. Pat. Nos. 6,194,551; to alter ability of the antibody to fix complement. WO 94/29351; and/or to increase antibody dependent cellular cytotoxicity (ADCC) and/or to increase the affinity of the antibody for an Fcγ receptor, WO 00/42072 by Presta. Moreover, the binding sites on human IgG1 for FcγRI, FcγRII, FcγRIII and FcRn have been mapped and variants with improved binding have been described. Shields, R. L. et al., 2001 J. Biol. Chem. 276: 6591-6604.

The antibody can have a modified pattern of glycosylation, hypoglycosylation, modification by alternative carbohydrates, or no glycosylation. EP 1,176,195 by Hang et al.; PCT Pub. WO 03/035835 by Presta; Shields, R. L. et al., 2002 J. Biol. Chem. 277: 26733-26740); WO 99/54342 by Umana et al.; Umana et al., 1999 Nat. Biotech. 17: 176-180.

The antibody can be pegylated, e.g., to increase the biological (e.g., serum) half-life of the antibody. As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. EP 0 154 316 by Nishimura et al. and EP 0 401 384 by Ishikawa et al. Pegylation can be altered by the introduction of a non-natural amino acid. Deiters et al., J Am Chem Soc 125: 11782-11783, 2003; Wang et al., Science 301: 964-967, 2003; Wang et al., Science 292: 498-500, 2001; Zhang et al., Science 303: 371-373, 2004; U.S. Pat. No. 7,083,970.

The present disclosure thus encompasses any and all manner of antibody or immunoglobulin to Fbxo40, modification, fragment or variant thereof, or molecule mimicking the functionality and/or structure of an anti-Fbxo40 antibody. All documents cited herein are hereby incorporated by reference in their entirety.

The antibodies, antibody-like molecules and other molecules which bind specifically and/or selectively to Fbxo40 can be used to make immunoconjugates, in which case they are conjugated to another moiety.

Thus, in another aspect, the methods of present disclosure employ immunoconjugate agents that target Fbxo40 and which inhibit or down-modulate Fbxo40, including, but are not limited to, cytotoxic agents, anti-inflammatory agents, e.g., a steroidal or nonsteroidal inflammatory agent, or a cytotoxin antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (actinomycin), bleomycin, mithramycin, and anthramycin (AMC)), and anti-mitotic agents (e.g., vincristine and vinblastine).

The term “cytotoxin” or “cytotoxic agent” includes any agent that is detrimental (e.g., kills) to fibrotic tissue. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, and analogs or homologs thereof.

Immunoconjugates can be formed by conjugating (e.g., chemically linking or recombinantly expressing) antibodies to suitable therapeutic agents. Suitable agents include, e.g., a cytotoxic agent, a toxin, and/or a radioactive isotope. Toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides can be used, e.g., ²¹²Bi, ¹³¹I, ¹³¹In, ⁹⁰Y and ¹⁸⁶Re.

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). A ricin immunotoxin can be prepared. Vitetta et al., Science 238: 1098 (1987). Carbon-14-labeled 1-isothiocyanatobenzyl-3-methyldiethylene triaminepentaacetic acid (MX-DTPA) is an example chelating agent for conjugation of radionucleotide to the antibody (see, e.g., WO94/11026).

Various other antibodies, antibody-like molecules and other molecules, and variants and immunoconjugates thereof, which specifically and/or selectively bind to and antagonize Fbxo40 can be prepared by one of ordinary skill in the art.

siRNA, Other Inhibitory Nucleic Acids and the Like that Antagonize Fbxo40

An antagonist of Fbxo40 can also be an inhibitory nucleic acid, e.g., a short inhibitory RNA (siRNA). In one embodiment, the Fbxo40 antagonist employed in the methods of the present disclosure is a siRNA or other nucleic acid complementary to a Fbxo40 nucleic acid or gene (or anti-sense or portion thereof), or a recombinant expression vector encoding the nucleic acid (or anti-sense or portion thereof). As used herein, an “antisense” nucleic acid comprises a nucleotide sequence complementary to a “sense” nucleic acid encoding the Fbxo40 protein (e.g., complementary to the coding strand of a double-stranded DNA, complementary to an mRNA or complementary to the coding strand of a Fbxo40 gene). As used herein, an “siRNA or antisense nucleic acid” and the like, in various contexts, can comprise any siRNA (double-stranded RNA) or any single-stranded DNA. As used herein and as detailed below, the term “siRNA” can encompass any type of RNA comprising a double-stranded region capable of mediating RNA interference (including molecules comprising two separate strands, two strands connected with a loop [e.g., a hairpin], two strands wherein one or both strands comprise a single-stranded nick, molecules comprising modified nucleotides and/or end caps, etc.). In one embodiment, the siRNA to Fbxo40 binds in the portion of the gene representing the Fbox. In another particular specific embodiment, the siRNA to Fbxo40 binds in the portion of the gene representing the zinc finger domain. In another particular specific embodiment, the siRNA binds to a portion of the gene which does not represent the Fbox. In another particular specific embodiment, the siRNA binds to a portion of the gene.

The use of antisense nucleic acids to down-modulate the expression of a particular protein in a cell is well known in the art. Weintraub et al. Reviews—Trends in Genetics, Vol. 1 1986; Askari et al. 1996 N. Eng. J. Med. 334: 316-318; Bennett et al. 1995 Circulation 92: 1981-1993; Mercola et al. 1995 Cancer Gene Ther. 2: 47-59; Rossi 1995 Br. Med. Bull. 51: 217-225; Wagner 1994 Nature 372: 333-335.

A siRNA or an antisense nucleic acid comprises a sequence complementary to, and is capable of hydrogen binding to, the coding strand of another nucleic acid (e.g., an mRNA). Antisense sequences complementary to an mRNA can be complementary to the coding region, the 5′ or 3′ untranslated region of the mRNA, and/or a region bridging the coding and untranslated regions, and/or portions thereof. Furthermore, a siRNA or an antisense nucleic acid can be complementary to a regulatory region of the gene encoding the mRNA, for instance a transcription or translation initiation sequence or regulatory element. Preferably, an antisense nucleic acid can be complementary to a region preceding or spanning the initiation codon on the coding strand or in the 3′ untranslated region of an mRNA.

siRNAs and antisense nucleic acids can be designed according to the rules of Watson and Crick base pairing. The siRNA or antisense nucleic acid molecule can be complementary to the entire coding region of Fbxo40 mRNA, but more preferably is an oligonucleotide which is antisense to only a portion of the coding or noncoding region of Fbxo40 mRNA. For example, the siRNA or antisense oligonucleotide can be complementary to the region surrounding the translation start site of Fbxo40 mRNA. A siRNA or an antisense nucleic acid can be, for example, about 5, 10, 15, 20, 25, 30, 35, 40, 45 or 50 nucleotides in length.

A siRNA or an antisense nucleic acid can be constructed using chemical synthesis and enzymatic ligation reactions using procedures known in the art. For example, a siRNA or an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The siRNA or anti-sense nucleic acid can also have an alternative backbone such as locked nucleic acids (LNA), Morpholinos, peptidic nucleic acids (PNA), threose nucleic acid (TNA), or glycol nucleic acid (GNA), and/or it can be labeled (e.g., radiolabeled or otherwise tagged). WO 2005/075637; WO 9518820; Zhang et al. 2005 J. Am. Chem. Soc. 127: 4174-5; Orgel 2000 Science 290 (5495): 1306-1307; Moulton 2009 Molecules 14: 1304-1323; Summerton 1999 Biochimica et Biophysica Acta 1489: 141-58.

In yet another embodiment, the siRNA or antisense nucleic acid molecule employed by the methods of the present disclosure can include an α-anomeric nucleic acid molecule. An α-anomeric nucleic acid molecule forms specific double-stranded hybrids with complementary RNA in which, contrary to the usual β-units, the strands run parallel to each other. Gaultier et al. 1987 Nucleic Acids. Res. 15: 6625-6641. The siRNA or antisense nucleic acid molecule can also comprise a 2′-o-methylribonucleotide (Inoue et al. 1987 Nucleic Acids Res. 15: 6131-6148) or a chimeric RNA-DNA analogue (Inoue et al. 1987 FEBS Lett. 215: 327-330).

In still another embodiment, an siRNA or antisense nucleic acid is a ribozyme. Ribozymes are catalytic RNA molecules with ribonuclease activity which are capable of cleaving a single-stranded nucleic acid, such as an mRNA, to which they have a complementary region. Thus, ribozymes [e.g., hammerhead ribozymes (described in Haselhoff et al. 1988, Nature 334: 585-591)] can be used to catalytically cleave Fbxo40 mRNA transcripts to thereby inhibit translation of Fbxo40 mRNA.

Alternatively, gene expression can be inhibited by targeting nucleotide sequences complementary to the regulatory region of Fbxo40 (e.g., the promoter and/or enhancers) to form triple helical structures that prevent transcription of the Fbxo40 gene. See generally, Helene 1991 Anticancer Drug Des. 6(6): 569-84; Helene et al. 1992 Ann. N.Y. Acad. Sci. 660: 27-36; and Maher 1992, Bioassays 14(12): 807-15.

Alternatively, the siRNA or antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The siRNA or antisense nucleic acid molecules of the present disclosure are typically administered to a subject or generated in situ such that they hybridize with cellular mRNA and/or genomic DNA encoding Fbxo40, and inhibit expression by inhibiting transcription and/or translation. An example of a route of administration of antisense nucleic acid molecules includes direct injection at a tissue site. Alternatively, siRNA or antisense nucleic acid molecules can be modified to target selected cells and then administered systemically. For example, for systemic administration, siRNA or antisense molecules can be modified such that they specifically bind to receptors or antigens expressed on a selected cell surface, e.g., by linking the nucleic acid molecules to peptides or antibodies which bind to cell surface receptors or antigens. The nucleic acid molecules can also be delivered to cells using vectors well known in the art and described in, for example, US20070111230, the entire contents of which are incorporated herein. To achieve sufficient intracellular concentrations of the molecules, vector constructs in which the nucleic acid molecule is placed under the control of a strong pol II or pol III promoter are preferred.

In another embodiment, a siRNA or an antisense nucleic acid used in the methods of the present disclosure is a compound that mediates RNAi (RNA interference). RNA interfering agents include, but are not limited to, nucleic acid molecules including RNA molecules which are homologous to Fbxo40 or a fragment thereof, “short interfering RNA” (siRNA), “short hairpin”, “small hairpin RNA” (shRNA), “microRNA” (miRNA) and other small molecules which modulate, interfere with or inhibit expression of a target gene by RNA interference (RNAi) at the level of gene regulation or mRNA transcription.

RNA interference is a post-transcriptional, targeted gene-silencing technique that uses double-stranded RNA (dsRNA) to degrade messenger RNA (mRNA) containing the same sequence as the dsRNA. Zamore et al. 2000 Cell 101: 25-33; Fire et al. 1998 Nature 391: 806; Hamilton et al. 1999 Science 286: 950-951; Lin et al. 1999 Nature 402: 128-129; Sharp 1999 Genes & Dev. 13: 139-141; Strauss 1999 Science 286: 886; Sharp et al. 2000 287: 2431-2432; Tuschl et al. 1999 Genes Dev. 13: 3191-3197.

The process of RNAi occurs when ribonuclease III (Dicer) cleaves the longer dsRNA into shorter fragments called siRNAs. siRNAs (small interfering RNAs) are typically about 21 to 23 nucleotides long and comprise about 19 base pair duplexes. Bass 2000 Cell 101: 235; Zamore et al. 2000 Cell 101: 25-33; Hammond et al. 2000 Nature 404: 293; Berstein et al. 2001 Nature 409: 363; Elbashir et al. 2001 Genes Dev. 15: 188). The smaller RNA segments then mediate the degradation of the target mRNA.

Dicer has also been implicated in the excision of 21- and 22-nucleotide small temporal RNAs (stRNAs) from precursor RNA of conserved structure that are implicated in translational control. Hutvagner et al. 2001, Science, 293, 834. The RNAi response also features an endonuclease complex, commonly referred to as an RNA-induced silencing complex (RISC), which mediates cleavage of single-stranded mRNA complementary to the antisense strand of the siRNA. Cleavage of the target RNA takes place in the middle of the region complementary to the antisense strand of the siRNA duplex. Elbashir et al. 2001 Genes Dev. 15: 188.

Kits for synthesis of RNAi are commercially available from, e.g., New England Biolabs and Ambion.

RNAi has been studied in a variety of systems. Fire et al. 1998 Nature 391: 806; Bahramian and Zarbl 1999 Mol. Cell. Biology 19: 274-283; Wianny and Goetz 1999 Nature Cell Biol. 2: 70; Hammond et al. 2000 Nature 404: 293; Elbashir et al. 2001 Nature 411: 494 and Tuschl et al. International PCT Publication No. WO 01/75164, describe RNAi induced by introduction of duplexes of synthetic 21-nucleotide RNAs in cultured mammalian cells including human embryonic kidney and HeLa cells.

Recent work in Drosophila embryonic lysates (Elbashir et al. 2001 EMBO J. 20: 6877 and Tuschl et al. International PCT Publication No. WO 01/75164) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Substitution of the 3′-terminal siRNA overhang nucleotides with 2′-deoxy nucleotides (2′-H) was tolerated. In addition, a 5′-phosphate on the target-complementary strand of a siRNA duplex is required for siRNA activity. Nykanen et al. 2001 Cell 107: 309.

Replacing the 3′-terminal nucleotide overhanging segments of a 21-mer siRNA duplex having two-nucleotide 3′-overhangs with deoxyribonucleotides does not have an adverse effect on RNAi activity. Replacing up to four nucleotides on each end of the siRNA with deoxyribonucleotides has been well tolerated, whereas complete substitution with deoxyribonucleotides results in no RNAi activity. Elbashir et al. 2001, EMBO J., 20, 6877 and Tuschl et al. International PCT Publication No. WO 01/75164. Li et al. International PCT Publication No. WO 00/44914, and Beach et al. International PCT Publication No. WO 01/68836 preliminarily suggest that siRNA may include modifications to either the phosphate-sugar backbone or the nucleoside to include at least one of a nitrogen or sulfur heteroatom. Kreutzer et al. Canadian Patent Application No. 2,359,180, also describe certain chemical modifications for use in dsRNA constructs in order to counteract activation of double-stranded RNA-dependent protein kinase PKR, specifically 2′-amino or 2′-O-methyl nucleotides, and nucleotides containing a 2′-0 or 4′-C methylene bridge.

Parrish et al. 2000 Molecular Cell 6: 1077-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in vitro such that interference activities could not be assayed. Id. at 1081. The authors also tested certain modifications at the 2′-position of the nucleotide sugar in the long siRNA transcripts and found that substituting deoxynucleotides for ribonucleotides produced a substantial decrease in interference activity, especially in the case of Uridine to Thymidine and/or Cytidine to deoxy-Cytidine substitutions. Id. In addition, the authors tested certain base modifications, including substituting, in sense and antisense strands of the siRNA, 4-thiouracil, 5-bromouracil, 5-iodouracil, and 3-(aminoallyl)uracil for uracil, and inosine for guanosine. Whereas 4-thiouracil and 5-bromouracil substitution appeared to be tolerated, Parrish reported that inosine produced a substantial decrease in interference activity when incorporated in either strand. Parrish also reported that incorporation of 5-iodouracil and 3-(aminoallyl)uracil in the antisense strand resulted in a substantial decrease in RNAi activity as well.

Those skilled in the art will appreciate that it is possible to synthesize and modify the siRNA as desired, using any conventional method known in the art (see Henschel et al. 2004 DEQOR: a web-based tool for the design and quality control of siRNAs. Nucleic Acids Research 32 (Web Server Issue): W113-W120). Further, it will be apparent to those skilled in the art that there are a variety of regulatory sequences (for example, constitutive or inducible promoters, tissue-specific promoters or functional fragments thereof, etc.) which are useful for the antisense oligonucleotide, siRNA, or shRNA expression construct/vector.

There are several examples in the art describing sugar, base, phosphate and backbone modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren 1992 TIBS. 17: 34; Usman et al. 1994 Nucleic Acids Symp. Ser. 31: 163; Burgin et al. 1996 Biochemistry 35: 14090). Sugar modification of nucleic acid molecules have been extensively described in the art [see Eckstein et al., International Publication PCT No. WO 92/07065; Perrault et al. Nature 1990 344: 565-568; Pieken et al. Science 1991 253: 314-317; Usman et al. Trends in Biochem. Sci. 1992 17: 334-339; Usman et al. International Publication PCT No. WO 93/15187; Sproat, U.S. Pat. No. 5,334,711 and Beigelman et al. 1995 J. Biol. Chem. 270: 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al. 1998 Tetrahedron Lett. 39: 1131; Earnshaw et al. 1998 Biopolymers (Nucleic Acid Sciences) 48: 39-55; Verma and Eckstein 1998 Annu. Rev. Biochem. 67: 99-134; and Burlina et al. 1997 Bioorg. Med. Chem. 5, 1999-2010; Iwase et al. 2007 Nucleosides, Nucleotides, and Nucleic Acids 26: 1451-1454; Iwase et al. Peptide Science 2003; M. Ueki, ed., The Japanese Peptide Society, Osaka, 2004, pp. 445-446; Elbashir et al. 2001 Nature 411: 494-498; Dowler et al. 2006 Nucl. Acids Res. 34: 1669-1675; Mesmaeker et al. 1996 Angew. Chem. Int. Ed. Engl. 35: 2790-2794; Rozners et al. 1997 Nucleosides Nucleotides 16: 967-970; Robins et al. 2000 Nucleosides, Nucleotides Nucleic Acids 19: 69-86; all of the references are hereby incorporated in their totality by reference herein]. Another example of modifications to siRNA in order to alter or improve their effectiveness is described by Hohjoh in FEBS Letters 557 (2004) pp. 193-198.

Additional modifications and 3′-endcaps are provided in WO 2005/021749 and WO 2007/128477.

Soutschek et al. 2004 Nature 432: 173-178 presented conjugation of cholesterol to the 3′ end of the sense strand of a siRNA molecule by means of a pyrrolidine linker, thereby generating a covalent and irreversible conjugate.

Chemical modifications (including conjugation with other molecules) of siRNA may also be made to improve the in vivo pharmacokinetic retention time and efficiency. Mark et al. 2006 Molecular Therapy, 13: 644-670.

Other general literature describing siRNA modifications includes: Chemical modification of siRNAs for in vivo use. Behlke 2008 Oligonucleotides 18: 305-19; Watts et al. 2008 Drug Discov. Today 13: 842-55; Peek et al. Curr. Opin. Mol. Ther. 2007 9: 110-8; Chen et al. 2005 Drug Discov. Today. 10: 587-93.

The use of longer dsRNA has been described. For example, Beach et al. International PCT Publication No. WO 01/68836, describes attenuating gene expression using endogenously-derived dsRNA. Tuschl et al. International PCT Publication No. WO 01/75164, describe a Drosophila in vitro RNAi system and the use of specific siRNA molecules for certain functional genomic and certain therapeutic applications. Li et al. International PCT Publication No. WO 00/44914, describe the use of specific long (141 bp-488 bp) enzymatically synthesized or vector expressed dsRNAs for attenuating the expression of certain target genes. Zernicka-Goetz et al. International PCT Publication No. WO 01/36646, describe certain methods for inhibiting the expression of particular genes in mammalian cells using certain long (550 bp-714 bp), enzymatically synthesized or vector expressed dsRNA molecules. Fire et al. International PCT Publication No. WO 99/32619, describe particular methods for introducing certain long dsRNA molecules into cells for use in inhibiting gene expression in nematodes. Plaetinck et al. International PCT Publication No. WO 00/01846, describe certain methods for identifying specific genes responsible for conferring a particular phenotype in a cell using specific long dsRNA molecules. Mello et al. International PCT Publication No. WO 01/29058, describe the identification of specific genes involved in dsRNA-mediated RNAi. Pachuck et al. International PCT Publication No. WO 00/63364, describe certain long (at least 200 nucleotide) dsRNA constructs. Deschamps Depaillette et al. International PCT Publication No. WO 99/07409, describe specific compositions consisting of particular dsRNA molecules combined with certain anti-viral agents. Waterhouse et al. International PCT Publication No. 99/53050 and 1998, PNAS, 95, 13959-13964, describe certain methods for decreasing the phenotypic expression of a nucleic acid in plant cells using certain dsRNAs. Driscoll et al. International PCT Publication No. WO 01/49844, describe specific DNA expression constructs for use in facilitating gene silencing in targeted organisms.

Others have reported on various RNAi and gene-silencing systems. For example, Parrish et al. 2000, Molecular Cell 6: 1077-1087 describes specific chemically modified dsRNA constructs targeting the unc-22 gene of C. elegans. Grossniklaus, International PCT Publication No. WO 01/38551, describes certain methods for regulating polycomb gene expression in plants using certain dsRNAs. Churikov et al. International PCT Publication No. WO 01/42443, describe certain methods for modifying genetic characteristics of an organism using certain dsRNAs. Cogoni et al, International PCT Publication No. WO 01/53475, describe certain methods for isolating a Neurospora silencing gene and uses thereof. Reed et al. International PCT Publication No. WO 01/68836, describe certain methods for gene silencing in plants. Honer et al. International PCT Publication No. WO 01/70944, describe certain methods of drug screening using transgenic nematodes as Parkinson's Disease models using certain dsRNAs. Deak et al. International PCT Publication No. WO 01/72774, describe certain Drosophila-derived gene products that may be related to RNAi in Drosophila. Arndt et al. International PCT Publication No. WO 01/92513 describe certain methods for mediating gene suppression by using factors that enhance RNAi. Tuschl et al. International PCT Publication No. WO 02/44321, describe certain synthetic siRNA constructs. Pachuk et al. International PCT Publication No. WO 00/63364, and Satishchandran et al. International PCT Publication No. WO 01/04313, describe certain methods and compositions for inhibiting the function of certain polynucleotide sequences using certain long (over 250 bp), vector expressed dsRNAs. Echeverri et al. International PCT Publication No. WO 02/38805, describe certain C. elegans genes identified via RNAi. Kreutzer et al. International PCT Publications Nos. WO 02/055692, WO 02/055693, and EP 1144623 B1 describes certain methods for inhibiting gene expression using dsRNA. Graham et al. International PCT Publications Nos. WO 99/49029 and WO 01/70949, and AU 4037501 describe certain vector expressed siRNA molecules. Fire et al. U.S. Pat. No. 6,506,559, describe certain methods for inhibiting gene expression in vitro using certain long dsRNA (299 bp-1033 bp) constructs that mediate RNAi. Martinez et al. 2002, Cell, 110, 563-574, describe certain single-stranded siRNA constructs, including certain 5′-phosphorylated single-stranded siRNAs that mediate RNA interference in HeLa cells. Harborth et al. 2003, Antisense & Nucleic Acid Drug Development, 13, 83-105, describe certain chemically and structurally modified siRNA molecules. Chiu and Rana, 2003, RNA, 9, 1034-1048, describe certain chemically and structurally modified siRNA molecules. Woolf et al. International PCT Publication Nos. WO 03/064626 and WO 03/064625 describe certain chemically modified dsRNA constructs.

siRNAs of the present disclosure can be delivered (e.g., to a cell in vitro or to a patient) by any means known in the art.

Delivery of siRNA to tissue is a problem both because the material must reach the target organ and must also enter the cytoplasm of target cells. RNA cannot penetrate cellular membranes, so systemic delivery of naked siRNA is unlikely to be successful. RNA is quickly degraded by RNAse activity in serum. For these reasons, other mechanisms to deliver siRNA to target cells has been devised. Methods known in the art include but are not limited to: viral delivery (retrovirus, adenovirus, lentivirus, baculovirus, AAV); liposomes (Lipofectamine, cationic DOTAP, neutral DOPC) or nanoparticles (cationic polymer, PEI), bacterial delivery (tkRNAi), and also chemical modification (LNA) of siRNA to improve stability.

Xia et al. 2002 Nat. Biotechnol. 20 and Devroe et al. 2002. BMC Biotechnol. 2 1: 15, disclose incorporation of siRNA into a viral vector.

Liposomes have been used previously for drug delivery (e.g., delivery of a chemotherapeutic). Liposomes (e.g., cationic liposomes) are described in PCT publications WO02/100435A1, WO03/015757A1, and WO04029213A2; U.S. Pat. Nos. 5,962,016; 5,030,453; and 6,680,068; and U.S. Patent Application 2004/0208921. A process of making liposomes is also described in WO04/002453A1. Furthermore, neutral lipids have been incorporated into cationic liposomes (e.g., Farhood et al. 1995). Cationic liposomes have been used to deliver siRNA to various cell types (Sioud and Sorensen 2003; U.S. Patent Application 2004/0204377; Duxbury et al., 2004; Donze and Picard, 2002). Use of neutral liposomes disclosed in Miller et al. 1998, and U.S. Patent Application 2003/0012812.

Chemical transfection using lipid-based, amine-based and polymer-based techniques, is disclosed in products from Ambion Inc., Austin, Tex.; and Novagen, EMD Biosciences, Inc, an Affiliate of Merck KGaA, Darmstadt, Germany); Ovcharenko D (2003) “Efficient delivery of siRNAs to human primary cells.” Ambion TechNotes 10 (5): 15-16). Additionally, Song et al. (Nat Med. published online (Fete I 0, 2003) doi: 10.1038/nm828) and others [Caplen et al. 2001 Proc. Natl. Acad. Sci. (USA), 98: 9742-9747; and McCaffrey et al. Nature 414: 34-39] disclose that liver cells can be efficiently transfected by injection of the siRNA into a mammal's circulatory system.

A variety of molecules have been used for cell-specific siRNA delivery. For example, the nucleic acid-condensing property of protamine has been combined with specific antibodies to deliver siRNAs. Song et al. 2005 Nat Biotch. 23: 709-717. The self-assembly PEGylated polycation polyethylenimine (PEI) has also been used to condense and protect siRNAs. Schiffelers et al. 2004 Nucl. Acids Res. 32: eI49, 141-1 10.

The siRNA-containing nanoparticles were then successfully delivered to integrin-overexpressing tumor neovasculature. Hu-Lieskovan et al. 2005 Cancer Res. 65: 8984-8992.

Other references disclosing siRNA delivery methodologies may be found in: Whitehead et al. 2009 Nat. Rev. Drug Discov. 8: 129-38; Wullner et al. 2009 Recent Pat. Anticancer Drug Discov. 4: 1-8; Aigner et al. 2008 Curr. Pharm. Des. 14(34): 3603-19; Kim et al. 2009 Pharm Res. 26: 657-66. Epub 2008 Nov. 18; Shen et al. IDrugs. 2008 August; 11(8): 572-8; Reischl et al. 2009 Nanomedicine. 2009 5: 8-20. Epub 2008 Jul. 18; Durcan et al. 2008 Mol Pharm. 5: 559-66; Sanguino et al. 2008 Mini Rev. Med. Chem. 8: 248-55; de Fougerolles et al. 2008 Hum. Gene Ther. 2008 19: 125-32; Akhtar et al. 2007 J. Clin. Invest. 117: 3623-32; Zhang et al. 2007 J. Control. Release 123: 1-10. Epub 2007 Aug. 7; Meade et al. 2007 Adv. Drug Deliv. Rev. 59: 134-40. Epub 2007 Mar. 15; Lewis et al. 2007 Adv. Drug Deliv. Rev. 59: 115-23. Epub 2007 Mar. 15; Sioud 2005 Expert Opin. Drug Deliv. 2: 639-51.

The design of a specific siRNA can involve an analysis of the mRNA secondary structure. mRNA in vivo is not linear; rather, it folds upon itself in a complex manner, forming double-stranded regions (e.g., stems) and single-stranded regions (e.g., loops). It can also form triple-stranded regions, pseudo-knots and other structures. Thus, an mRNA can have multiple paired and unpaired segments and assorted hairpin structures. Methods have been proposed for predicting this secondary structure of mRNAs. Zuker 2003 Nucl. Acids Res. 31: 3406-15; and Mathews et al. 1999 J. Mol. Biol. 288: 911-940. Methods have also been proposed for predicting which siRNA would bind in single-stranded regions of an mRNA. WO 2005/075637.

siRNAs that are particularly useful for this disclosure include those which can bind specifically to a region of the Fbxo40 mRNA, and have one or more of the following qualities: binding in the coding segment of Fbxo40; binding at or near the junction of the 5′ untranslated region and the start of the coding segment; binding at or near the translational start site of the mRNA; binding at or near junctions of exons and introns; little or no binding to the mRNAs of other genes (little or no “off-target effects”); binding to the Fbxo40 mRNA in or near a region or regions that is not double-stranded or a stem region, e.g., in a loop or single-stranded portion; eliciting little or no immunogenicity; binding in a segment of the Fbxo40 mRNA sequence which is conserved among various animal species (including human, mouse, rat, cynomolgus monkey, etc.), as the presence of a conserved sequence facilitates testing using various laboratory animals; binding to double-stranded region(s) of the mRNA; binding to an AT-rich region (e.g., at least about 50, 51, 52, 53, 54, 55, 56, 57, 58, 59, or 60% AT-rich); and lacking particular sequences known or suspected to decrease siRNA activity, e.g., the presence of a GG sequence at the 5′ end, which may decrease separation of the double-stranded portion of the siRNA.

siRNA may have modifications internally, or at one or both ends. The modifications at the ends can help stabilize the siRNA, protecting it from degradation by nucleases in the blood. The siRNAs may optionally be directed to regions of the Fbxo40 mRNA known or predicted to be near or at splice sites of the gene; e.g., exon-intron junctions. The siRNAs can also optionally be designed to anneal to known or predicted exposed and/or single-stranded regions of the mRNA (e.g., loops).

As noted above, the mRNA sequence for human Fbxo40 is readily available, e.g., at GenBank: NM_{—}016298 (SEQ ID NO: 2). The sequences for several animal homologues are available: Mouse (Mus musculus): NM_{—}001037321; Rat (Rattus norvegicus): XM_{—}344023; Chimpanzee (Pan troglodytes): NC_{—}006490.2; Rhesus macaque (Macaca mulatta): NC_{—}007859.1; Zebrafish (Danio rerio): BX322577.11 (pseudogene) or XP_{—}694708.3; Wild boar (Sus scrofa): EU743742; Chicken (Gallus gallus): XP_{—}424000.2; and Dog (Canis lupus familiaris): XP_{—}545126.2.

siRNAs can be designed as Fbxo40 antagonists which bind to and assist in degradation of Fbxo40 mRNA. The anti-Fbxo40 siRNAs can be designed to bind to the coding segment or non-coding segment (e.g., the 5′ or 3′ untranslated regions, or UTRs). Preferably the siRNA binds to the coding segment of the mRNA. The siRNAs can have double-stranded regions of, for example, 17, 18, 19, 20, 21, 22, 23, or 24 bp. Preferably the siRNA comprises 19 or 21 bp. The siRNAs can also have overhangs of 0, 1, or 2 overhangs; preferably, as in the case of 0 nt overhangs, they are blunt-ended. The mRNA sequence of a gene may vary from individual to individual, especially at wobble positions within the coding segment, or in the untranslated region; individuals may also differ from each other in coding sequence, resulting in additional differences in mRNA and corresponding siRNA sequence. siRNAs can also be modified in sequence to reduce immunogenicity, binding to undesired genes (e.g., “off-target effects”) or to increase stability in the blood. (These sequence variants are independent of chemical modification of the bases or 5′ or 3′ or other end-caps of the siRNAs.)

From the sequence presented as SEQ ID NO: 2, suitable sequences of siRNAs comprising the 19-mer and an overhang can be readily determined. The anti-sense strand is readily deduced, as above, based on Watson-Crick pairing. Overhangs can be added based on the full gene sequence provided above, in SEQ ID NO: 2.

In addition, in one particular specific embodiment, the Fbxo40 siRNA(s) comprise an overhang of dTdT on either or both 3′ ends.

In addition, in various particular specific embodiments, the Fbxo40 siRNAs comprise a double-stranded region comprising any portion of 15, 16, 17, or 18 nt of SEQ ID NO: 1.

In one particular specific embodiment, the selected Fbxo40 siRNA(s) consist of the 19-mer sequences of the siRNAs which start at positions nt 1 to 5704, along with the anti-sense strand.

In another particular specific embodiment, the selected Fbxo40 siRNA(s) comprise the 19-mer sequences of the siRNAs which start at positions nt 1 to 5704, along with the anti-sense strand, but in addition, have overhangs on one or the other strand. In another particular specific embodiment, the selected Fbxo40 siRNA(s) have overhangs on both strands. In another particular specific embodiment, the selected Fbxo40 siRNA(s) have an overhang on only one strand. The overhang(s) can be at the 3′ or 5′ end. In another particular specific embodiment, the selected Fbxo40 siRNA(s) have overhang(s) which are less than 5 nt long. In another particular specific embodiment, the selected Fbxo40 siRNA(s) haveoverhang(s) which are 2 nt long.

In another embodiment, the Fbxo40 siRNAs comprise any siRNA which begins at any sequence from 1 to 5709, but is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46 nt in length. In one particular specific embodiment, the siRNA comprises a double-stranded region of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46 nt in length. In one especially particular specific embodiment, the Fbxo40 siRNA(s) comprise a double-stranded region of 19 or 21 bp.

In one particular specific embodiment, the selected Fbxo40 siRNA(s) comprise a 19-mer with a perfect match between the human and mouse homologues. This facilitates the use of the mouse as an animal model.

In another particular specific embodiment, the selected Fbxo40 siRNA(s) comprise a 19-mer with a perfect match between the human and rat homologues. This facilitates the use of the rat as an animal model.

In another particular specific embodiment, the selected Fbxo40 siRNA(s) comprise a 19-mer with a perfect match between the human and Sus scrofa homologues.

In another particular specific embodiment, the selected Fbxo40 siRNA(s) comprise a 19-mer with a perfect match between the human and chicken homologues.

In another particular specific embodiment, the selected Fbxo40 siRNA(s) comprise a 19-mer with a perfect match between the human and Macaca homologues.

In another particular specific embodiment, the selected Fbxo40 siRNA(s) comprise a 19-mer with a perfect match between the human and Pan homologues.

In another particular specific embodiment, the selected Fbxo40 siRNA(s) match in sequence between the human, rat and Macaca mulatta homologues.

In another particular specific embodiment, the selected Fbxo40 siRNA(s) comprise a 19-mer with a perfect match between the human, mouse, and Macaca homologues.

In another particular specific embodiment, the selected Fbxo40 siRNA(s) comprise a 19-mer with a perfect match between the human, mouse, rat, Sus, Pan and Macaca homologues.

In another particular specific embodiment, the selected Fbxo40 siRNA(s) comprise a 19-mer with a perfect match between the human, mouse, rat, and Macaca homologues.

The present disclosure further comprises sequences which represent a portion (e.g., 15, 16, 17, or 18 nt long) of the recited 19-mers. Thus, for example, a 19-mer with a sequence matching the human Fbxo40 and a particular animal comprises various 15, 16, 17, and 18-mer sequences which can also be used in this disclosure.

In one particular specific embodiment, the selected Fbxo40 siRNA(s) target the sequences of CACCTCCTGGAAAGTCCACAA (SEQ ID NO: 19), GTGGGAAAGTATGTTCAGCAA (SEQ ID NO: 20) or AGCCGTGGATGCCAAAGACTA (SEQ ID NO: 21) (or the RNA equivalents thereof).

In another particular specific embodiment, the selected Fbxo40 siRNA(s) comprise a sequence which is unique to the human Fbxo40 gene (thus, not found in another human gene). This will reduce off-target effects.

In another particular specific embodiment, the selected Fbxo40 siRNA(s) bind to particular secondary structures in the Fbxo40 mRNA. mRNAs are known to form complex secondary structures, comprising double-stranded regions (e.g., stems) and single-stranded regions (e.g., loops). Other structures (e.g., pseudo-knots and triple-stranded regions) are also possible. These structures can be predicted from a known sequence by various software. Zuker 2003 Nucl. Acids Res. 31: 3406-15; and Mathews et al. 1999 J. Mol. Biol. 288: 911-940.

As a non-limiting example, the following parameters can be used: Folding temperature: 37° C. The RNA sequence is linear. Ionic conditions: 1M NaCl, no divalent ions. Percent suboptimality number: 5. Upper bound on number of computed folding: 50. Window parameter: Default. Maximum interior/bulge loop size: 30. Maximum asymmetry of an interior/bulge loop: 30. Maximum distance between paired bases: no limit.

Without wishing to be bound by a particular theory, the Inventors note that particular structures with an mRNA may be particularly amenable to binding to siRNAs. These areas are designated “hotspots.” Methods have also been proposed for predicting which siRNA would bind in single-stranded regions of an mRNA. WO 2005/075637.

These structures include single-stranded regions (e.g., loops); sequences comprising short loops (e.g., a 19- or 21-nt siRNA comprising one or two shorter loops interspersed with stem regions); sequences adjacent to loops, particularly sequences directly downstream of a loop (e.g., with a loop 5′ to the sequence that binds the siRNA); sequences spanning two stem regions. Thus a siRNA useful as a Fbxo40 antagonist may bind to one or more single-stranded regions (or portion thereof), one or more double-stranded regions (or portion thereof), or may bind adjacent to one or two single-stranded regions. Furthermore, sequences that are highly conserved across species lines can also be very amenable to siRNA. In addition, mRNA sequences known to be bound by host proteins may not be.

Thus, the Fbxo40 antagonist of the present disclosure can include, without limitation, a siRNA that (a) corresponds to (and anneals to) at least one, two or more predicted loops in the Fbxo40 mRNA (corresponding or annealing to portions or entireties of the loops); (b) is adjacent to one or two predicted loops in the Fbxo40 mRNA; (c) corresponds to at least one, two or more stem structures; and/or (d) lies adjacent to one or two stem structures of the Fbxo40 mRNA. Preferably, the siRNA anneals to a Fbxo40 mRNA sequence predicted to comprise a loop comprising at least about 1, 2, 3, 4, 5, 6, 7, 8, 9, or 10 nucleotides, more preferably at least about 4, even more preferably at least about 6. In another particular specific embodiment, the siRNA anneals to a Fbxo40 sequence adjacent to a loop comprising at least about 1, 2, 3, 4, 5, 6, 7, 8, 9, or 10 nucleotides, more preferably at least about 4, even more preferably at least about 6.

In addition to the disclosure herein, any method or material known in the art can be used to prepare an inhibitory nucleic acid, siRNA, or the like that is capable of antagonizing Fbxo40.

### Antagonizing Fbxo40

The Fbxo40 antagonist (whether comprising a LMW, antibody, siRNA or other composition) will decrease the activity, level and/or expression of Fbxo40.

Any method known in the art can be use to measure changes in Fbxo40 activity, level and/or expression induced by a Fbxo40 antagonist. Measurements can be performed at multiple timepoints, prior to, during and after administration of the antagonist, to determine the effect of the antagonist.

The level or expression of Fbxo40 can be measured by evaluation of mRNA (e.g., via Northern blots or PCR), or protein (e.g., Western blots). The effect of an antagonist on Fbxo40 expression can be determined by measuring Fbxo40 gene transcription rates (e.g., via Northern blots; or reverse transcriptase polymerase chain reaction or real-time polymerase chain reaction). RT-PCR has been used to show that mRNA levels of Fbxo40 are high in kidney, pancreas and prostate, and medium in liver and spleen. Brauner-Osborne et al. 2001. Biochim. Biophys. Acta 1518: 237-248. Direct measurements can be made of levels of Fbxo40, e.g. by Western blots of tissues in which Fbxo40 is expressed, including the heart and skeletal muscle.

Several avenues are available for measuring Fbxo40 activity. Fbxo40 activity can be measured by Fbxo40's ability to bind to IRS1. Alternatively, Fbxo40 activity can be measured by the protein's ability to bind to Skp1.

Such evaluations can measure the down-regulation of Fbxo40 expression, level or activity mediated by a Fbxo40 antagonist.

A method of screening compositions for the ability to increase muscle mass or prevent, limit or reduce the loss of muscle mass in an individual, comprising:
ascertaining the level or activity of Fbxo40 in a cell,
treating the cell with a composition, and
ascertaining the level or activity of Fbxo40 in the cell again,
   wherein an ability of the composition to decrease the level or activity of Fbxo40 is correlated to the ability to increase muscle mass or prevent the loss of muscle mass in an individual.

In this method, the level or activity of Fbxo40 can be measured in a cell in vitro. The level or expression of Fbxo40 can be measured using any method known in the art, e.g., those discussed above, such as measuring the mRNA or protein level of Fbxo40. The activity of Fbxo40 can be measured by any method known in the art, e.g., those discussed above, such as measuring the ability of Fbxo40 to interact with Skp1 and/or IRS1.

The cell can then be treated with a putative Fbxo40 antagonist. Several cells of the same type can be treated with different levels of the putative antagonist or a control (such as PBS, phosphate buffered saline). The cell can also or alternatively be treated multiple times with the putative antagonist.

The cells can then be re-measured for Fbxo40 level, expression or activity.

In addition, methods for using adenoviruses to deliver a siRNA antagonist, and then measuring the efficacy of the siRNA on promoting muscle hypertrophy, are known in the art.

As a non-limiting example, high titer and purified adenoviruses expressing a siRNA antagonist to the human Fbxo40 can be generated. The adenoviruses can be titered. Mouse primary myoblasts are isolated, grown and differentiated as described previously. Primary myoblasts are seeded, for example, at 8×10⁵ cells/well in 6-well plates or 4×10⁵ cell/well in 12-well plates. They are induced to differentiate the next day. Two days post-differentiation, myotubes are transduced with various adenovirues as indicated. Cells are subjected to various analyses at 48 or 72 hr post-transduction as indicated. The titers used for transduction can be, for example, 2.5×10⁸ or 1×10⁹ particle particle/ml.

A non-limiting example of use of an adenovirus to deliver and test the efficacy of a siRNA on antagonizing Fbxo40 is presented here. Primary myotubes can be, for example, transduced with adenovirus, e.g., for 48 hrs. Media is removed and cells washed with PBS for three times. They can be fixed, for example, with 5% glutaraldehyde for 20 minutes at 37′C, followed by two washes with PBS. Cell morphology can be examined, for example, using a Zeiss Axovert microscope set at 10× magnification. Once the focused image was obtained, the microscope can be set to FITC for fluorescent imaging. Random images can be captured from each well and saved for analysis. The images can be analyzed, e.g., using the Pipeline Pilot Webport to obtain the myotube thickness as an indicative of muscle size. Statistical analysis can be performed on all the data, e.g., using two-tailed Student's t test. A p value less than 0.05, for example, can be considered significant.

Additional tests for the efficacy of a Fbxo40 antagonist (alone or in combination with other treatments) in treating muscle loss can be determined by measurements of muscle mass. Such measurements can be performed by means known in the art. For testing of rats and other laboratory animals, leg muscles (e.g., soleus, tibialis anterior and gastrocnemius) can be removed and examined. Denervation, immobilization, and unweighting in rats all result in similar rates of loss in mass of the medial gastrocnemius muscle. Bodine et al. Science 2001 294: 1704-1707. Cachexia can also be induced in rats by administration of interleukin-1 (IL-1) and the glucocorticoid dexamethsaone. Bodine et al. 2001. In addition, nude mice implanted with OCC-1 cells are useful animal models for cachexia for testing various compositions of the present disclosure.

For human patients, repetitive or timed physical activities can be used to measure muscle mass. Total appendicular skeletal mass (ASM) can also be measured in accordance with Gallagher et al. 1997 J. Appl. Physiol. 83: 229-239; and Baumgartner et al. 1998. Axial skeletal muscle mass of a patient can be determined by dxa (dual energy X-ray absorptiometry) or a similar measure.

One strategy for evaluating a Fbxo40 antagonist is treating patients with anterior cruciate ligament (ACL) repair after traumatic rupture. These patients undergo above-the-knee casting for an extended period post-operatively, often leading to substantial quadriceps atrophy. The contralateral leg can serve as a comparator for atrophy in the placebo-treat group to validate the study. Mid-thigh muscle mass can be evaluated by DEXA (dual energy X-ray absorptiometry) and single repetition maximum strength assessment by quadriceps extension. Additional assessments include measurement of power of the calf. A positive outcome can include statistically significant preservation of quadriceps mass and strength in the drug-treated group compared to placebo.

Another method of testing muscle atrophy in vitro involves the use of myotubes. A myotube is a developing skeletal muscle fiber with a tubular appearance; it is a skeletal muscle fiber formed by the fusion of myoblasts during a developmental stage; a few myofibrils occur at the periphery, and the central core is occupied by nuclei and sarcoplasm so that the fiber has a tubular appearance. Differentiated, post-mitotic, multi-nucleate C2C12 skeletal myotubes, for example, Bains et al. 1984 Mol. Cell. Biol. 4: 1449, can be used. These can be infected with an adenovirus encoding a gene associated with atrophy (e.g., Fbxo40 along with Enhanced Green Fluorescent Protein (EGFP) or another marker), or with a control (adenovirus with EGFP alone). Immunoblots can be used to confirm infection and quantify levels of Fbxo40 and control protein expression. After a suitable time (e.g., 2 days), myotube diameters can be measured, the myotubes infected with adenovirus carrying the Fbxo40 gene being found to be thinner. Growth with myotubes infected adenovirus carrying Fbxo40 can be used to test the efficacy of the antagonist in suppressing the ability of Fbxo40 to mediate atrophy.

These various tests of muscle mass can be repeated over time evaluate muscle state; or before or after treatment to evaluate the efficacy of a treatment regimen, and the regimen adjusted according to patient response. Baumgartner et al. 1998 defined sarcopenia as a state wherein the patient has an ASM less than two standard deviations below the mean of a young reference group (the t-score).

An example of the present disclosure slowing the progression of sarcopenia would be to change the length of time a patient would go from a t-score of −1.5 to −2; e.g., if such a progression would normally take 5 years, then treatment as used herein could slow this change to 10 years. Examples of partial reversal include reducing a t-sore about 0.1, 0.2, 0.3, 0.4, 0.5, 0.6, 0.7, 0.8, 0.9, 1.0 or more units (e.g., moving from a t-score of −2 to a t-score of −1.9, −1.8, −1.6, −1.5, −1.4, −1.3, −1.2, −1.1, etc.). Treating sarcopenia also includes delaying the onset of sarcopenia. For example, if a typical male aged 50 would begin to see signs of sarcopenia by age 55, treatment according to the present disclosure would delay the onset about 1, 2, 3, 4, 5, 6, 7, 8, 9, 10 or more years.

In additional to ASM measurements, measurements can also be taken of body weight, fat, abdominal visceral fat, fat-free mass, leg ASM, thigh muscle ASM, body water and cell mass and muscle strength.

The ability of the antagonist to decrease Fbxo40 level, expression or activity is correlated with the ability of the antagonist to increase muscle mass, or prevent loss of muscle mass, in an individual.

### Screening for Fbxo40 Antagonists

The present disclosure also encompasses methods for screening compositions for usefulness in antagonizing Fbxo40 and increasing muscle mass or preventing, limiting or reducing the loss of a muscle mass in a patient.

In one specific embodiment, the present disclosure encompasses a method of screening compositions for the ability to increase muscle mass or prevent, limit or reduce the loss of muscle mass in an individual, comprising:
(a) ascertaining the level or activity of Fbxo40 in a cell from the individual,
(b) optionally, treating the cell with a composition comprising an antagonist to
Fbxo40, and
(c) optionally, ascertaining the level or activity of Fbxo40 in the cell again,
   wherein an elevated level of Fbxo40 relative to a control is an indication that the subject has or is at risk of developing a muscle-wasting disorder, and wherein an ability of the composition to decrease the level or activity of Fbxo40 is correlated to the ability to increase muscle mass or prevent the loss of muscle mass in an individual.

In one specific embodiment of this method, the individual is afflicted with a muscle wasting-associated selected from: cachexia, cancer, tumor-induced weight loss, sepsis, chronic heart failure, rheumatoid arthritis, acquired immune deficiency syndrome, sarcopenia, diabetes, hypertension, high levels of serum cholesterol, high levels of triglycerides, Parkinson's disease, insomnia, drug addiction, pain, insomnia, hypoglycemia, compromised liver function, cirrhosis, gall bladder disorders, chorea, dyskinesia, kidney disorder, and/or uremia.

In one specific embodiment of this method, the antagonist reduces the level, expression or activity of Fbxo40.

In various embodiments of this method, the antagonists that are screened can include a low molecular weight composition (LMW), an antibody or the like (e.g., Fbxo40 antibody, Fbxo40 antibody-like molecule, molecule which binds specifically and/or selectively to Fbxo40), and/or siRNA or other inhibitory nucleic acid, as described herein or otherwise known in the art.

As noted above, various other methods for creating and screening compound libraries of LMWs are described in, inter alia, U.S. Pat. Nos. 6,764,858; 6,723,235; 6,720,190; 6,677,160; 6,656,739; 6,649,415; 6,630,835; 6,627,453; 6,617,114; 6,613,575; 6,607,921; 6,602,685; 6,448,794; 6,421,612; 6,395,169; 6,387,257; 6,355,163; 6,214,561; 6,187,923; and 6,054,047.

Any of these methods for creating and screening libraries of LMWs or any other method known to one of ordinary skill in the art can be used to obtain a small molecule that binds to and antagonized Fbxo40.

Methods of screening antibodies, antibody-like molecules, and/or molecules which bind specifically and/or selectively to a target, such as Fbxo40, are also known in the art.

Also known in the art are methods of screening siRNAs and other inhibitory nucleic acids to a target, such as Fbxo40.

Methods of ascertaining the level, activity or expression of Fbxo40 in a cell from an individual are known in the art. These methods can be used prior to and after exposure of the cell to a candidate antagonist to Fbxo40.

These methods of screening are useful for identifying compositions for the ability to antagonize Fbxo40 and increase muscle mass or prevent the loss of muscle mass, as described herein and known in the art.

Various terms in the embodiment of the disclosure related to screening antagonists for Fbxo40 and ability to prevent muscle loss or maintain muscle mass are defined herein.

### Diagnostic Methods

The present invention also provides novel methods for assessing whether a subject has or is at risk of developing a muscle-wasting disorder. Individuals suspected of having a muscle-wasting disorder would benefit from early detection, so that disease progression can be retarded or even halted or reversed. The methods include assessing whether a subject has or is at risk of developing a muscle-wasting disorder comprising contacting a sample from a subject with a reagent able to detect Fbxo40 and detecting Fbxo40, wherein an elevated level of Fbxo40 relative to a control is an indication that the subject has or is at risk of developing a muscle-wasting disorder.

The invention further provides methods for determining or predicting the efficacy of a treatment regimen for treating a muscle-wasting disorder. These methods include assessing the efficacy of a treatment regimen for treating a muscle-wasting disorder in a subject, the method comprising: a) contacting a first sample obtained from the subject prior to administering at least a portion of the treatment regimen to the subject with a reagent able to detect Fbxo40; b) contacting a second sample obtained from the subject following administration of at least a portion of the treatment regimen with a reagent able to detect Fbxo40; and c) comparing the levels of Fbxo40 from the first and second samples, wherein an elevated level of Fbxo40 present in the first sample, relative to the second sample, is an indication that the treatment regimen is efficacious for treating a muscle-wasting disorder in the subject.

As used herein, the term “sample” includes any body fluid (e.g., blood fluids, lymph, gynecological fluids, cystic fluid, urine, ocular fluids and fluids collected by peritoneal rinsing), or a cell from a subject. Normally, the tissue or cell will be removed from the patient, but in vivo diagnosis is also contemplated. Other patient samples, include tear drops, serum, cerebrospinal fluid, feces, sputum and cell extracts.

As used herein, the term “reagent able to detect Fbxo40” includes any agent capable of binding specifically with Fbxo40 and transforming Fbxo40 into a detectable moiety. Suitable reagents include antibodies, antibody derivatives, antibody fragments, and the like. Suitable reagents for binding with a Fbxo40 nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

As used herein, the term “control” can be the level of Fbxo40 in a sample from a subject not suffering from a muscle-wasting disorder. It can be the same type of sample as the test sample or different. For example, if the sample from the subject being tested is a heart or muscle sample (e.g., a cell, a collection of cells, or tissue obtained from a heart or muscle biopsy), then the control sample can also be a heart or muscle sample from a subject not suffering from a muscle-wasting disorder. Alternatively, the control sample can be of a different type, e.g., it can be a sample from a subject not suffering from a muscle-wasting disorder. In other embodiments, the control sample can be a collection of samples from a subject not having a muscle-wasting disorder or a sample from a collection of subjects not have a muscle-wasting disorder.

As used herein, “an aberrant level” of Fbxo40 is any level of Fbxo40 that differs from the control level of Fbxo40, e.g., significantly higher or elevated levels.

As used herein, a “higher level,” “elevated level,” or “increased level” of Fbxo40 refers to a level that is elevated relative to a suitable control. Preferably, the differential from the suitable control is greater than the standard error of the assay employed to assess the level. Moreover, the elevated level is preferably at least twice, and more preferably three, four, five, six, seven, eight, nine or ten times the level of Fbxo40 in a suitable control (e.g., a sample from a subject not having a fibrotic disease or the average level of Fbxo40 in several control samples or other suitable benchmark).

As used herein, the terms “efficacious” and “efficacy” refers to the likelihood that a treatment regimen will treat a muscle-wasting disorder in a subject. For example, a treatment regimen is deemed “efficacious” and considered a viable treatment option if the treatment leads to an alleviation of the a muscle-wasting disorder symptoms (e.g., muscle loss, loss of appetite, weakness, compromised immune function and/or electrolyte imbalance) in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

### A. Assays

The presence, absence, and/or level of Fbxo40 in a biological sample obtained from a subject may be assessed by any of a wide variety of in vitro and in vivo techniques and methods, which transform Fbxo40 within the sample into a moiety that can be detected and quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, enzyme linked immunosorbent assays (ELISAs), immunoblotting, Western blotting, Northern blotting, electron microscopy, mass spectrometry, immunoprecipitations, immunofluorescence, Southern hybridizations and the like.

In one embodiment, the presence, absence, and/or level Fbxo40 in a sample can be assessed using a reagent, such as an antibody (e.g., a radio-labeled, chromophore-labeled, fluorophore-labeled, or enzyme-labeled antibody), an antibody derivative (e.g., an antibody conjugated with a substrate or with the protein or ligand of a protein-ligand pair (e.g. biotin-streptavidin)), or an antibody fragment (e.g., a single-chain antibody or an isolated antibody hypervariable domain) which binds specifically to and transforms the biomarker, e.g., Fbxo40, in a sample into a detectable molecule.

The term “labeled”, with regard to the antibody, is intended to encompass direct labeling of the antibody by coupling (i.e., physically linking) a detectable substance to the antibody, as well as indirect labeling of the antibody by reactivity with another reagent that is directly labeled. Examples of indirect labeling include detection of a primary antibody using a fluorescently labeled secondary antibody, such that it can be detected with fluorescently labeled streptavidin.

In another embodiment, the presence, absence, and/or level of Fbxo40 is assessed using a nucleic acid. For example, in one embodiment, the presence, absence, and/or level of Fbxo40 is assessed using a nucleic acid probe.

The term “probe”, as used herein, refers to any molecule that is capable of selectively binding to Fbxo40. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to Fbxo40 mRNA. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to Fbxo40 genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of Fbxo40 mRNA.

An alternative method for determining the level of Fbxo40 mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, Fbxo40 expression is assessed by quantitative fluorogenic RT-PCR (i.e., the TaqMan™ System). Such methods typically utilize pairs of oligonucleotide primers that are specific for Fbxo40. Methods for designing oligonucleotide primers specific for a known sequence are well known in the art.

The expression levels of Fbxo40 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of Fbxo40 expression may also comprise using nucleic acid probes in solution.

In one embodiment of the invention, microarrays are used to detect Fbxo40 expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Furthermore, in vivo techniques for detection of Fbxo40 include introducing into a subject a labeled antibody directed against Fbxo40, which binds to and transforms Fbxo40 into a detectable molecule. As discussed above, the presence, level, or even location of the detectable Fbxo40 in a subject may be detected determined by standard imaging techniques.

In another embodiment, mass spectrometry can be used to detect Fbxo40 in a sample. Mass spectrometry is an analytical technique that consists of ionizing chemical compounds to generate charged molecules (or fragments thereof) and measuring their mass-to-charge ratios. In a typical mass spectrometry procedure, a sample is obtained from a subject, loaded onto the mass spectrometry, and its components (e.g., Fbxo40) are ionized by different methods (e.g., by impacting them with an electron beam), resulting in the formation of charged particles (ions). The mass-to-charge ratio of the particles is then calculated from the motion of the ions as they transit through electromagnetic fields.

### A. Diagnostic Assays

The presence, absence, and/or level of Fbxo40 may be assessed by any of a wide variety of well known methods for detecting a molecule or protein. Non-limiting examples of such methods include immunological methods for detection of proteins, protein purification methods, protein function or activity assays, nucleic acid hybridization methods, nucleic acid reverse transcription methods, and nucleic acid amplification methods, ELISA, immunoblotting, Western blotting, Northern blotting, Southern blotting and the like.

In one embodiment, the presence, absence, and/or level of Fbxo40 is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair (e.g. biotin-streptavidin)), or an antibody fragment (e.g. a single-chain antibody, an isolated antibody hypervariable domain, etc.) which binds specifically to the biomarker, i.e., Fbxo40, such as the protein encoded by the open reading frame corresponding to the biomarker or such a protein which has undergone all or a portion of its normal post-translational modification. The term“labeled”, with regard to the antibody, is intended to encompass direct labeling of the antibody by coupling (i.e., physically linking) a detectable substance to the antibody, as well as indirect labeling of the antibody by reactivity with another reagent that is directly labeled. Examples of indirect labeling include detection of a primary antibody using a fluorescently labeled secondary antibody, such that it can be detected with fluorescently labeled streptavidin. In another embodiment, the presence, absence, and/or level of Fbxo40 is assessed using a nucleic acid.

The detection methods of the invention can be used to detect Fbxo40, for example, in a biological sample in vitro as well as in vivo. For example, in vitro techniques for detection of mRNA include Northern hybridizations, in situ hybridizations and QPCR. In vitro techniques for detection of Fbxo40 include, for example, enzyme linked immunosorbent assays (ELISAs), Western blots, immunoprecipitations and immunofluorescence. In vitro techniques for detection of Fbxo40 DNA include, for example, Southern hybridizations. Furthermore, in vivo techniques for detection of Fbxo40 include introducing into a subject a labeled antibody directed against Fbxo40. As discussed herein, the antibody can be labeled with a radioactive biomarker whose presence and location in a subject can be detected by standard imaging techniques.

### Additional Definitions

In order to provide a clear understanding of the specification and claims, the following additional definitions are conveniently provided below.

The present disclosure provides an antagonist of Fbxo40 (and methods of making and use the same) for administration to patients at risk for or suffering from a muscle wasting-associated disorder. The disclosure also encompasses treatments comprising an effective amount of the antagonist. The treatment can be supplied at various dosages, and can comprise, as a non-limiting example, a pharmaceutically acceptable salt and/or carrier.

By “patient” is meant an individual, preferably human, afflicted by or at risk for muscle-wasting-associated disorder, a disorder related to or associated with muscle loss. The patient in need of a medicament for preventing the loss of or increasing muscle mass may be afflicted by one or more afflictions only indirectly related, or not related at all, to muscle mass loss. The patient may be at risk for (e.g., genetically predisposed) for a muscle wasting-associated disorder, but may show few or no signs of disease (e.g., the disorder may be sub-clinical). In this case, the antagonist can be administered as a preventative treatment to prevent the development of muscle loss.

The patient can be treated with any appropriate treatment for these ailments, in addition to (prior to, simultaneous with, or after) treatment with a Fbxo40 antagonist. Thus, a patient can be treated with one or more Fbxo40 antagonists, optionally one or more additional treatments or medicaments that ameliorate muscle loss, and optionally one or more treatments for another disease (e.g., cancer, diabetes or Huntington's Disease). For example, a patient with diabetes can be administered a Fbxo40 antagonist and insulin; a cancer patient can be administered a Fbxo40 antagonist and an anti-cancer medicament. Muscle-wasting disorders, particularly in elderly patients, are sometimes associated with bone loss disorders such as osteoporosis. A Fbxo40 antagonist can thus also be co-administered with a treatment for osteoporosis, e.g., Aclasta (Zoledronic acid or zoledronate) or Denosumab (AMG 162; an antibody that targets RANKL).

The present disclosure further comprises treatments and treatment methods involving antagonists to Fbxo40 for administration to patients and individuals.

By “treatment” is meant prophylaxis, therapy, cure, or any other change in a patient's condition indicating improvement or absence of degradation of physical condition. By “treatment” is meant treatment of muscle loss or treatment of any other ailment the patient has. As used herein, the terms “treatment” and “treat” refer to both prophylactic or preventative treatment and curative or disease-modifying treatment, including treatment of patients at risk of contracting a disease or suspected of having a disease, as well as patients already ill or diagnosed as suffering from a condition. The terms “treatment” and “treat” also refer to the maintenance and/or promotion of health in an individual not suffering from a disease but who may be susceptible to developing an unhealthy condition, such as nitrogen imbalance or muscle loss.

An “effective amount” or a “therapeutically effective amount” is an amount that treats a disease or medical condition of an individual, or, more generally, provides a nutritional, physiological or medical benefit to an individual.

In various embodiments of the disclosure, the patient is at least about 6 months, 1, 2, 3, 4, 5, 6, 7, 8, 9, 10, 20, 30, 40, 50, 55, 60, 65, 70, or 75 years of age. In various embodiments, the patient is no more than about 10, 20, 30, 40, 50, 55, 60, 65, 70, 75, 80, 90, or 100 years of age. In various embodiments the patient has a body weight of at least about 90, 100, 120, 140, 160, 180, 200, 220, 240, 260, 280, 300, 320, 340, 360, 380 or 400 lbs. In various embodiments, the patient has a body weight of no more than about 90, 100, 120, 140, 160, 180, 200, 220, 240, 260, 280, 300, 320, 340, 360, 380 or 400 lbs.

In various embodiments of the disclosure, the dosage can be at least about 1, 5, 10, 25, 50, 100, 200, 250, 300, 250, 400, 450, 500, 550, 600, 650, 700, 750, 800, 850, 900, 950 or 1000 ng, 1, 5, 10, 25, 50, 100, 200, 250, 300, 250, 400, 450, 500, 550, 600, 650, 700, 750, 800, 850, 900, 950 or 1000 micrograms, 1, 5, 10, 25, 50, 100, 200, 250, 300, 250, 400, 450, 500, 550, 600, 650, 700, 750, 800, 850, 900, 950 or 1000 mg. In various embodiments, the dosage can be no more than about 10, 25, 50, 100, 200, 250, 300, 250, 400, 450, 500, 550, 600, 650, 700, 750, 800, 850, 900, 950 or 1000 mg. In various embodiments, the dosage can be administered at least more than once a day, daily, more than once a weekly, weekly, bi-weekly, monthly, every 2, 3, 4, 5, 6, 7, 8, 9, 10, 11 or 12 months.

In various embodiments, the dosage is correlated to the body weight or body surface area of the individual. The actual dosage level can be varied to obtain an amount of active agent which is effective for a particular patient, composition and mode of administration, without being toxic to the patient. The selected dose will depend on a variety of pharmacokinetic factors, including the activity of the particular antagonist employed, the route of administration, the rate of excretion of the antagonist, the duration of the treatment, other drugs, compounds and/or materials used in combination with the antagonist, the age, sex, weight, condition, general health and prior medical history of the patient, and like factors well known in the medical arts. A physician or veterinarian having ordinary skill in the art can readily determine and then prescribe the effective amount of the antagonist required. A suitable dose will be that amount which is the lowest dose effective to produce a therapeutic effect, or a dose low enough to produce a therapeutic effect without causing side effects.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p 1445, the disclosure of which is hereby incorporated by reference.

The pharmaceutical compositions comprising a Fbxo40 antagonist can be in solid form, for example, powders, granules, tablets, pills, gelcaps, gelatin capsules, liposomes, suppositories, chewable forms, or patches. The pharmaceutical compositions comprising a Fbxo40 antagonist can also be presented in liquid form, for example, solutions, emulsions, suspensions, elixirs, or syrups. Appropriate liquid supports can be, for example, water, organic solvents such as polyol, such as glycerol or glycols, including propylene glycol and polyethylene glycol, or ethanol, Cremophor EL, or mixtures thereof, in varying proportions, in water. The compositions can comprise nano-sized amorphous or crystalline granules coated with albumin or a surfactant.

Appropriate supports can include, for example, antibacterial and antifungal agents, buffering agents, calcium phosphate, cellulose, methyl cellulose, chlorobutanol, cocoa butter, colorings, dextrin, emulsifiers, enteric coatings, flavorings, gelatin, isotonic agents, lecithin, magnesium stearate, perfuming agents, polyalcohols such as mannitol, injectable organic esters such as ethyl oleate, paraben, phenol sorbic acid, polyethylene glycol, polyvinylpyrrolidine, phosphate buffered saline (PBS), preserving agents, propylene glycol, sodium carboxymethylcellulose, sodium chloride, sorbitol, various sugars (including, but not limited to, sucrose, fructose, galactose, lactose and trehalose), starch, suppository wax, talc, vegetable oils, such as olive oil and corn oil, vitamins, wax, and/or wetting agents. For Fbxo40 antagonists which are siRNAs, a particular specific support comprises dextran and water, e.g. 5% dextrose in water (D5W).

The biologically inert portion of the pharmaceutical composition can optionally be layered and/or erodible, allowing timed release of the antagonist.

The pharmaceutical composition comprising a Fbxo40 can be administered by buccal, inhalation (including insufflation and deep inhalation), nasal, oral, parenteral, implant, injection or infusion via epidural, intraarterial, intraarticular, intracapsular, intracardiac, intracerebroventricular, intracranial, intradermal, intramuscular, intraorbital, intraperitoneal, intraspinal, intrasternal, intrathecal, intravenous, subarachnoid, subcapsular, subcutaneous, subcuticular, transendothelial, transtracheal, transvascular, rectal, sublingual, topical, and/or vaginal route. This may be by injection, infusion, dermal patch, or any other method known in the art. The formulation can be powdered, nebulized, aerosolized, granulized or otherwise appropriately prepared for delivery. The administration, if liquid, may be slow or via bolus, though, under some circumstances known in the art, bolus injections may lead to loss of material through the kidneys.

The Fbxo40 antagonists can be administered with medical devices known in the art. For example, in a particular specific embodiment, an antagonist can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, antagonists can be formulated to ensure proper distribution in vivo. For example, the blood-brain barrier (BBB) excludes many highly hydrophilic compounds. To ensure that the Fbxo40 antagonists cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29: 685). Example targeting moieties include folate or biotin (see, e.g., U.S. Pat. No. 5,416,016 to Low et al.); mannosides (Umezawa et al., (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); surfactant protein A receptor (Briscoe et al. (1995) Am. J. Physiol. 1233: 134), different species of which may comprise the formulations of the disclosures, as well as components of the invented molecules; p120 (Schreier et al. (1994) J. Biol. Chem. 269: 9090); see also K. Keinanen; M. L. Laukkanen (1994) FEBS Lett. 346: 123; J. J. Killion; I. J. Fidler (1994) Immunomethods 4: 273.

In various embodiments, methods and compositions of this disclosure are as described herein, but with the proviso that the antagonist is not an antibody. In various embodiments, methods and compositions of this disclosure are as described herein, but with the proviso that the antagonist is not an antibody-like molecule. In various embodiments, methods and compositions of this disclosure are as described herein, but with the proviso that the antagonist is not a small organic compound (LMW). In various embodiments, methods and compositions of this disclosure are as described herein, but with the proviso that the antagonist is not a siRNA.

The articles “a” and “an” as used herein refer to one or more than one (at least one) of the grammatical object of the article.

The present disclosure is further illustrated by the following examples which should not be construed as further limiting. The contents of all figures and all references, patents and published patent applications cited throughout this application are expressly incorporated herein by reference.

### Example 1

### Upon IGF1 Treatment, IRS1 is Rapidly Degraded in C2C12 Myotubes

IGF1 has been demonstrated to be sufficient to induce hypertrophy in adult skeletal muscle. Insulin receptor substrates (IRS) are tyrosine-phosphorylated upon IGF1 or insulin-binding to the cognate receptor, thereby enabling them to form a signaling complex with many SH2-domain-containing proteins and initiate multiple intracellular signals. Alteration of IRS levels can affect the sensitivity and response to both IGF1 and insulin. In many different cell model systems, exposure to IGF1 or insulin leads to a reduction in IRS levels.

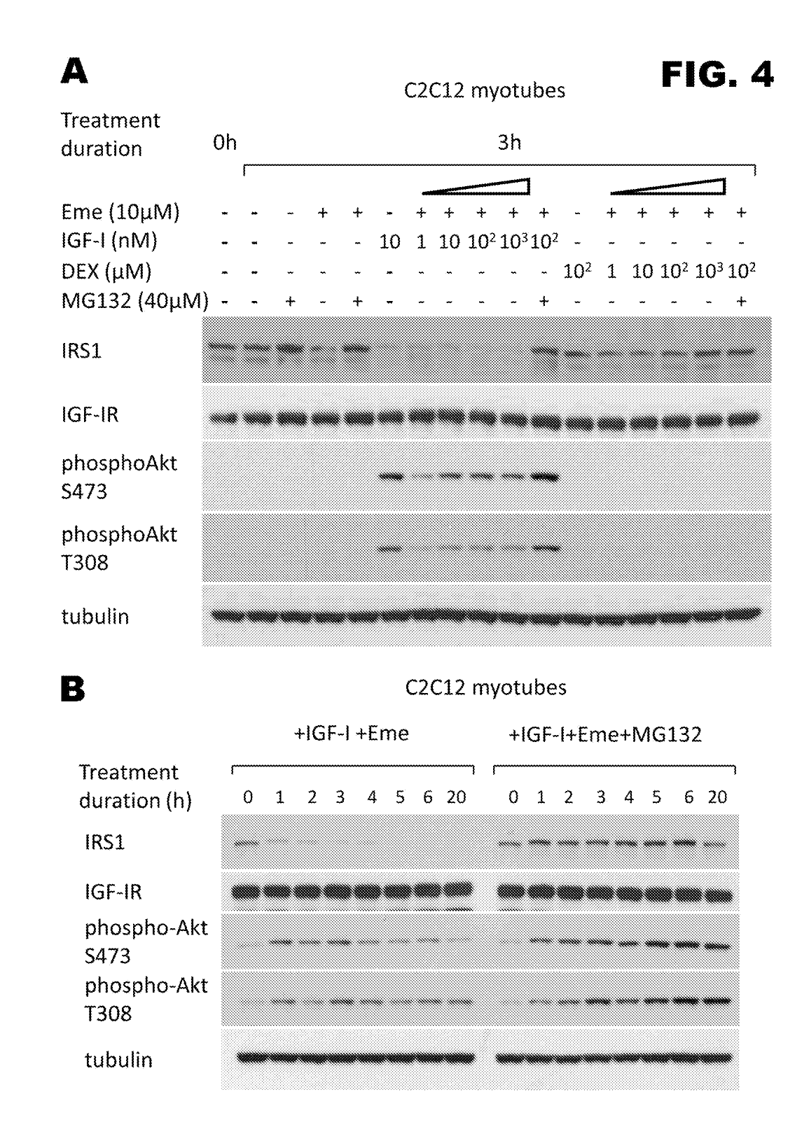

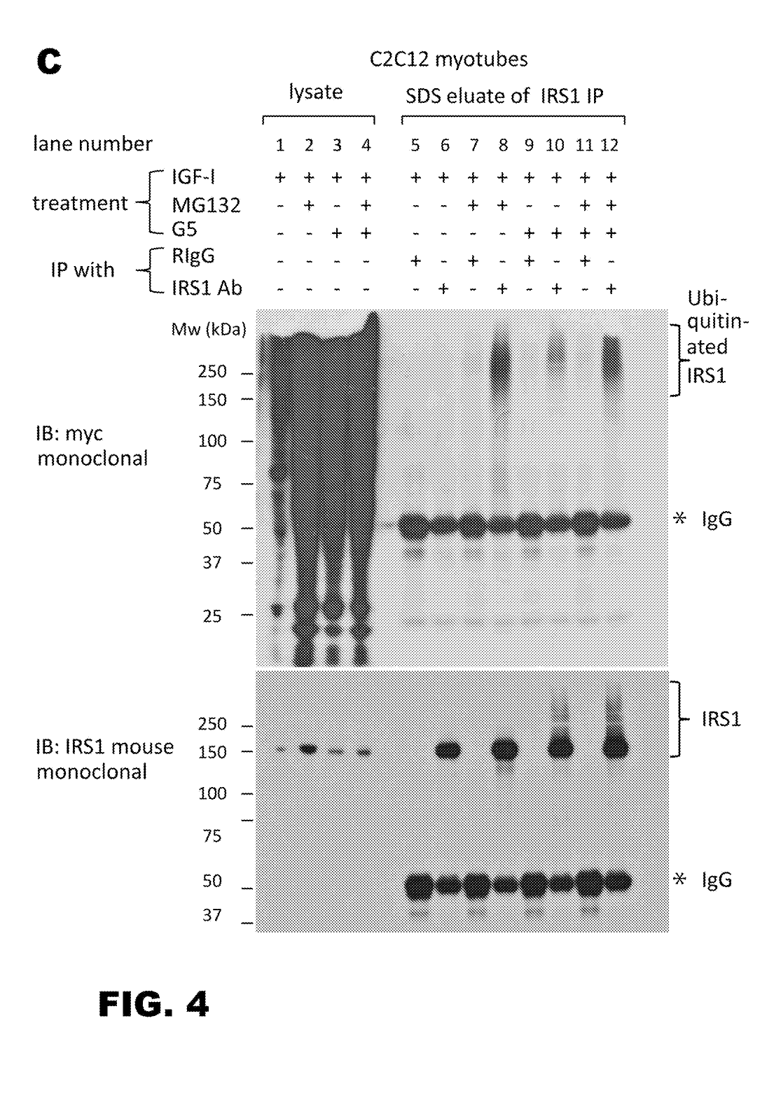

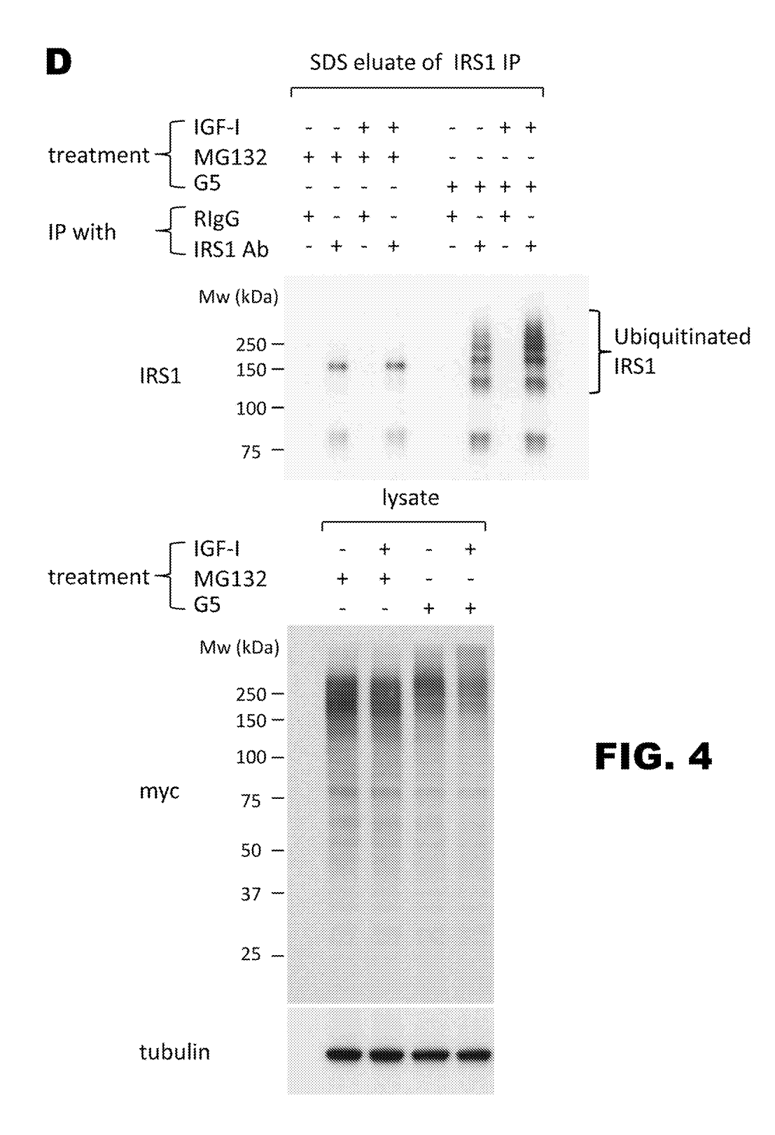

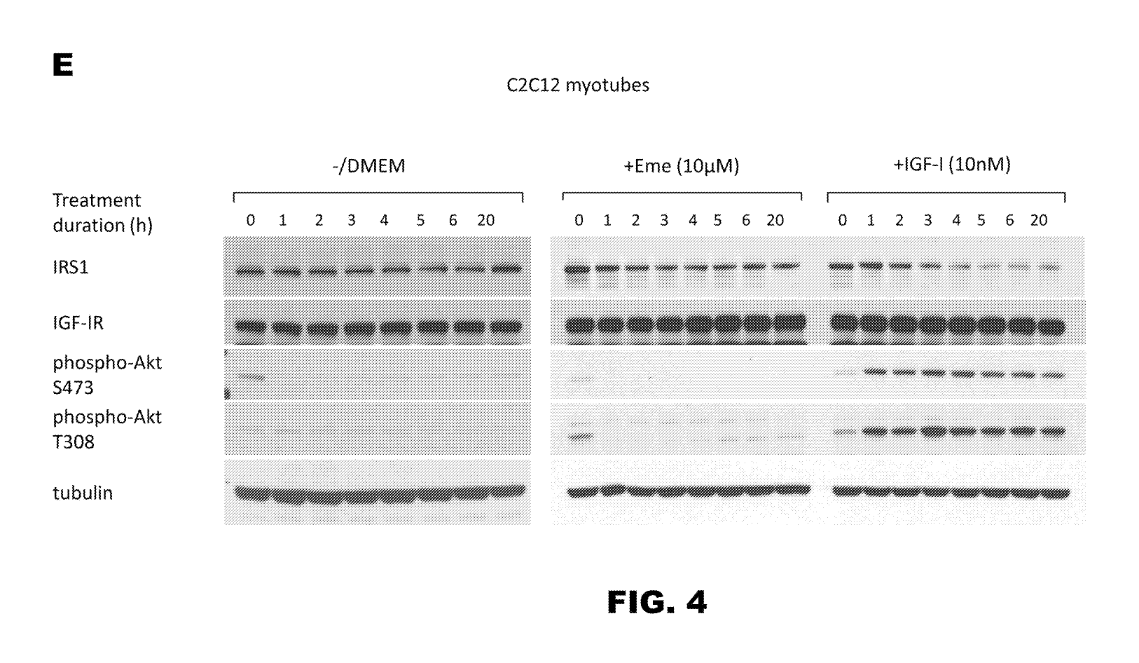

In this example, we check whether endogenous IRS1 is degraded in muscle cells upon IGF1 treatment. Differentiated C2C12 myotubes are treated with increasing concentration of IGF1 or dexmethasome (DEX), a glucocorticoid that can lead to muscle atrophy, along with protein synthesis inhibitor Emetin (Eme). FIG. 4A demonstrates that IRS1 is degraded upon IGF1 treatment, but not with DEX, in a dose-dependent manner in C2C12 myotubes. Based on this result, we choose to use 10 nM IGF1 for our future experiments. In C2C12 myotubes, IRS1 is rapidly degraded upon IGF1 treatment with a half-life of around 2 hours (FIGS. 4B and 4E). Note that the protein level of IRS1 also decreases in cells treated with IGF1 alone even though with a slower kinetics (FIG. 4E), suggesting that this rapid degradation surpasses active protein synthesis.

IRS1 is degraded through a proteosome dependent pathway. This conclusion is based on the observation that the proteosomal inhibitor MG132 substantially stabilizes this protein (FIG. 4B). Since ubiquitinated proteins are rapidly degraded in cells, in order to detect ubiquitinated IRS1 protein in the experiment shown in FIG. 4C, C2C12 myotubes are infected with an adenovirus overexpressing his-myc tagged ubiquitin and incubated with IGF1 along with MG132 or an isopeptidase inhibitor G5. IRS1 is immunoprecipitated with the rabbit polyclonal anti-IRS1 antibody (lanes 6, 8, 10, 12) or non-specific IgG (lanes 5, 7, 9, 11) as control. Poly-ubiquitination is detected with monoclonal anti-myc antibody against the his-myc tagged ubiquitin and is found to be specifically immunoprecipitated with IRS1 (FIG. 4C, upper panel). Interestingly, the upper shifts in electrophoretic mobility of IRS1 are only obvious with G5 treatment (FIG. 4C, lower panel, lanes 10 and 12), consistent with the inhibitory role of G5 on isopeptidase. Note that in the upper panel of FIG. 4C, G5 treatment also causes an upper shift of his-myc tagged ubiquitin in comparison to MG132 treatment alone (compare lane 10 to lane 8). In agreement with the induced degradation of IRS1 upon IGF1 treatment, poly-ubiquitinated IRS1 also increases with IGF1 treatment (FIG. 4D, upper panel) despite that the total amount of ubiquitinated proteins in cells even decreased a little (FIG. 4D, lower panel).

These data show that IGF1 leads to rapid degradation of IRS1 in C2C12 myotubes. Furthermore, IRS1 is degraded through a proteosome dependent pathway.

We next determine what enzymes are or are not involved in this degradation.

### Example 2

### The Degradation of IRS1 in C2C12 Myotubes Cannot be Rescued by Inhibitors to PI3-Kinase and mTOR

In order to determine which signaling pathways play a role in the activation of IRS1 degradation in C2C12 myotubes, myotubes are incubated with 10 nM IGF1 and inhibitors to PI3-kinase (wortmannin), Akt (API-2), GSK3 (LiCl), mTOR (rapamycin), MEK (PD98059 and MEK1/2 inhibitor), p38 and JNK or carrier (DMSO). The data (not shown) indicate that the degradation of IRS1 in C2C12 myotubes cannot be rescued by inhibitors to PI3-kinase and mTOR. This data suggests that the ligand induced degradation of IRS1 may due to the direct phosphorylation of IRS1 by IGF1R.

### Example 3

### IRS1 is Targeted by the Skp1-Cullin1-Rbx1 Complex

In this example, we try to find the E3 ligases that are responsible for targeting IRS1 to the proteosome for degradation. Since IRS1 has rapid degradation in C2C12 myotubes that is differently regulated than any other cell types reported so far in literature, we focus our search in C2C12 myotubes.

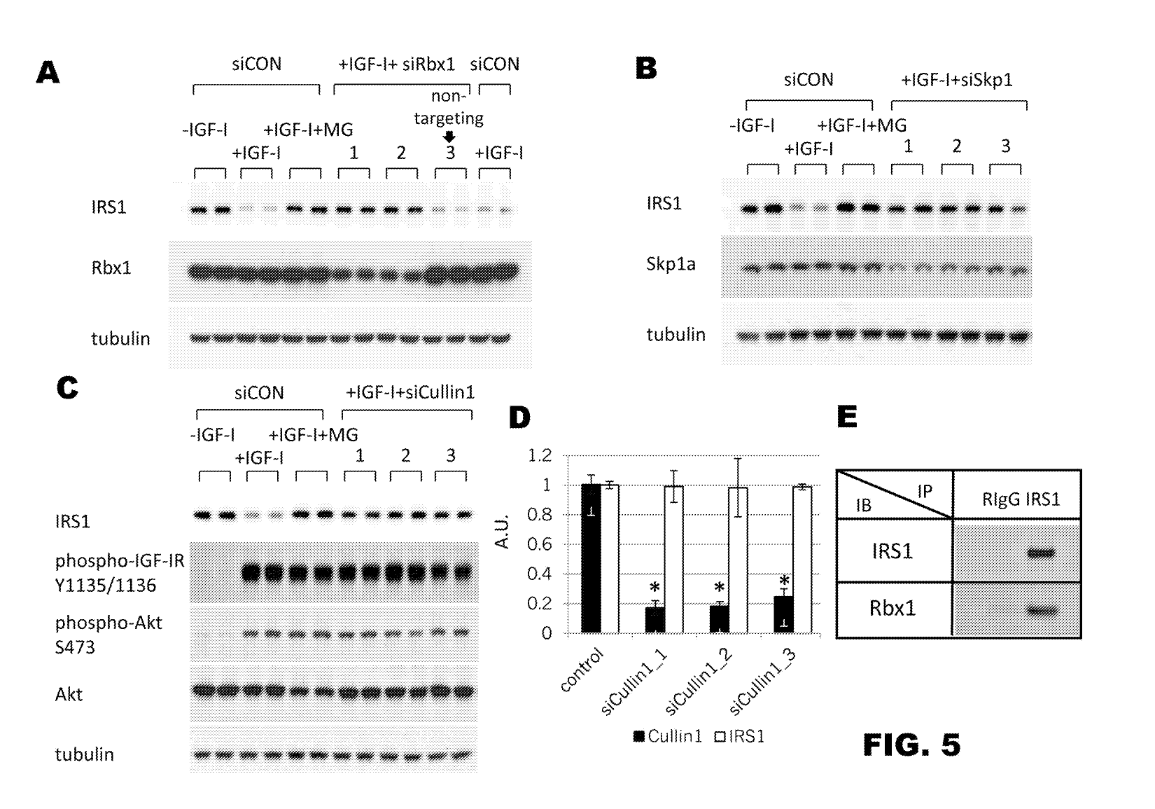

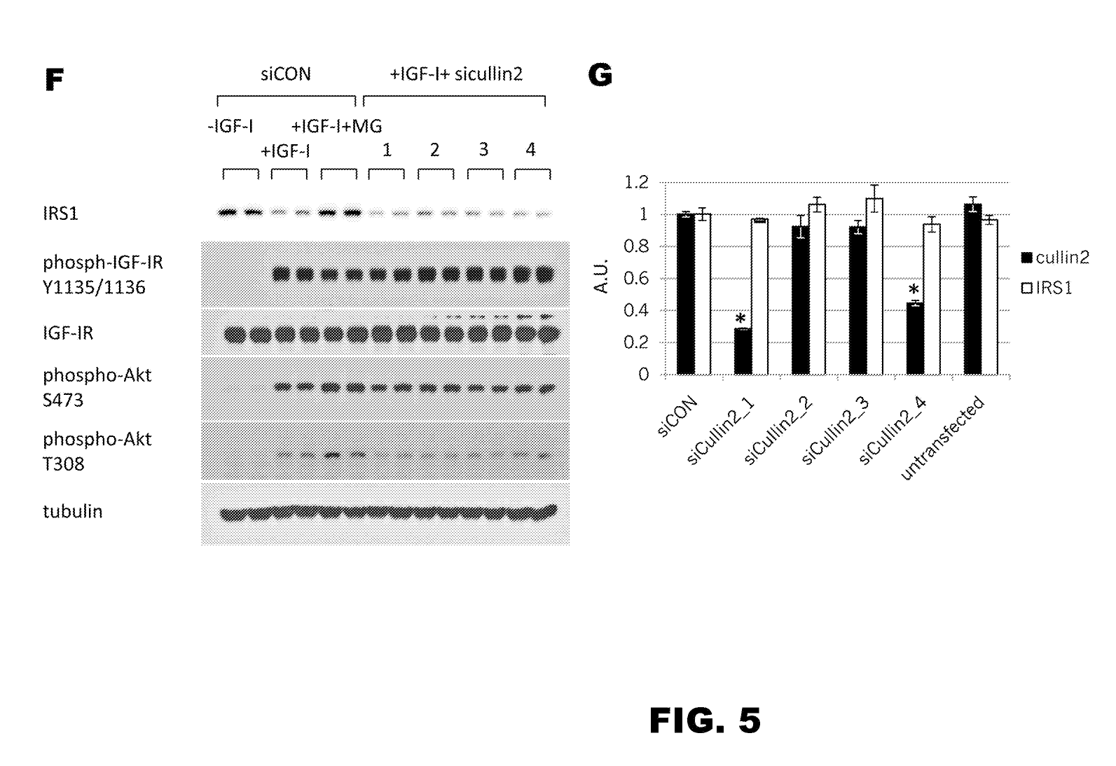

There are more than 600 E3 ligases in the cell [Li et al. 2008 PLoS One 3: e1487], which include two major types: Ring-finger domain (RNF) and HECT domain containing E3 ligases. RNF-dependent E3 ligases can be further divided into two categories: the single polypeptide chain RNF E3 and the multi-subunit cullin-Ring E3 complexes. Rbx1 is the common small RNF protein that can associate with various factors to form four different types of multi-subunit cullin-Ring E3 complexes. In order to quickly find the E3 ligases targeting IRS1, we first try to narrow down the potential targets by knocking down Rbx1. C2C12 myotubes are transfected with three different siRNAs targeting Rbx1. As shown in FIG. 5A, siRbx1_{—}1 and siRbx1_{—}2 knockdown Rbx1 protein to 50% of basal level and almost completely blocks IGF1 induced IRS1 degradation; while siRbx1_{—}3, which does not knockdown protein significantly, has no effect (FIG. 5A). Furthermore, siRNAs to Skp1 (FIG. 5B) and cullin1 (FIGS. 5C and 5D) have a similar effect, suggesting that the Rbx1 containing Skp1-cullin1-F-box (SCF) complex is the E3 ligase targeting IRS1 for proteosomal degradation. As a control, we also check the effect of cullin2 knockdown and find that cullin2 is not involved (FIGS. 5F and 5G). Finally, Rbx1 can be co-immunoprecipitated with IRS1, suggesting that these two proteins exist in one complex (FIG. 5E).

These data show that IRS1 is targeted by the Skp1-Cullin1-Rbx1 complex. Thus, one Fbox protein is involved in IGF1-induced IRS1 degradation. The next step is to determine which Fbox protein is involved.

### Example 4

### Fbxo40 Associates with Skp1-Cullin1-Rbx1 Complex and Targets IRS1 for Degradation

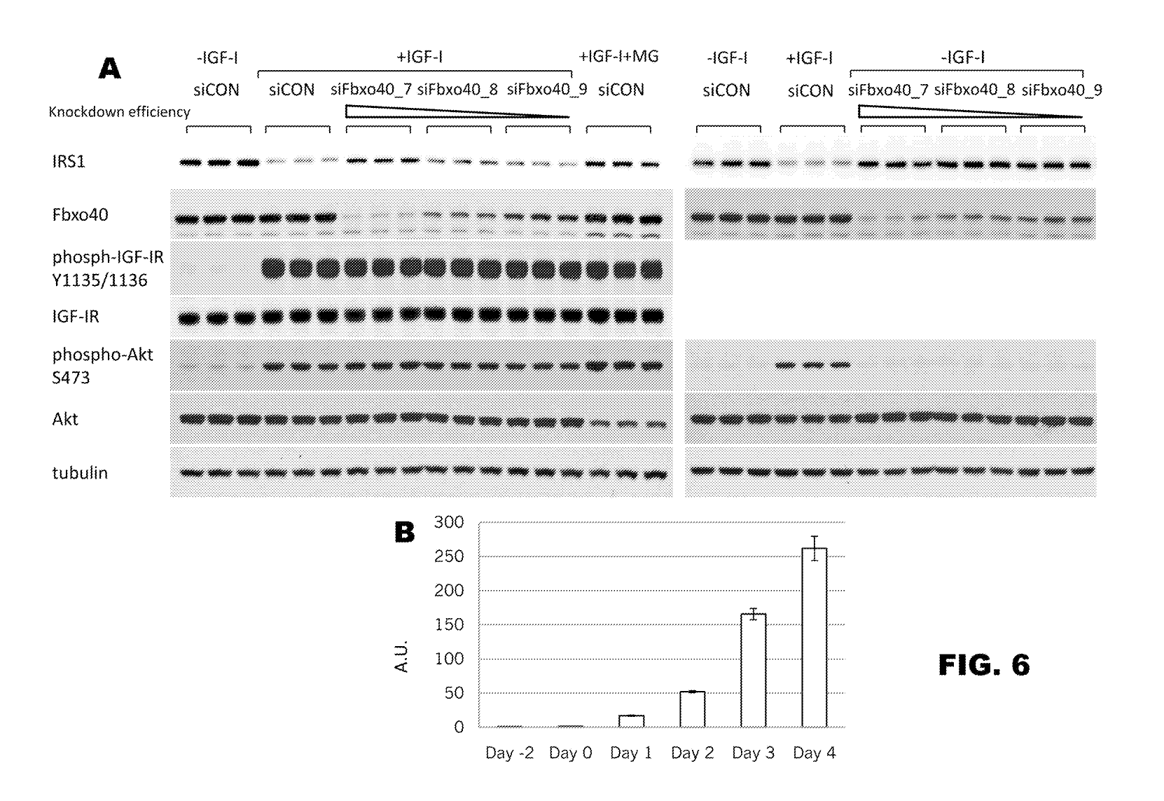

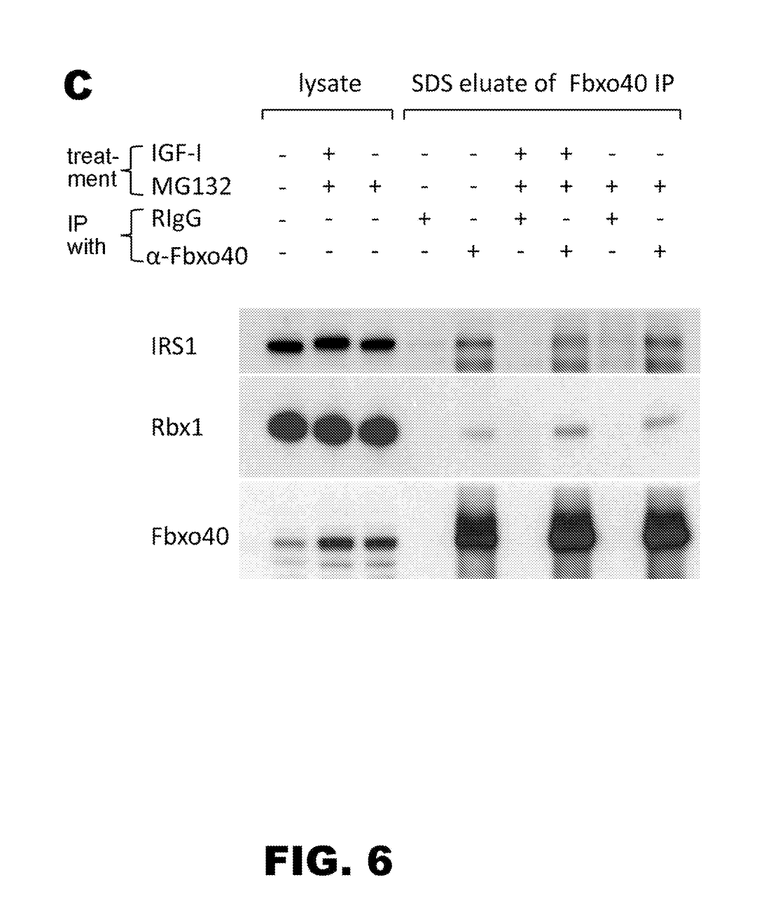

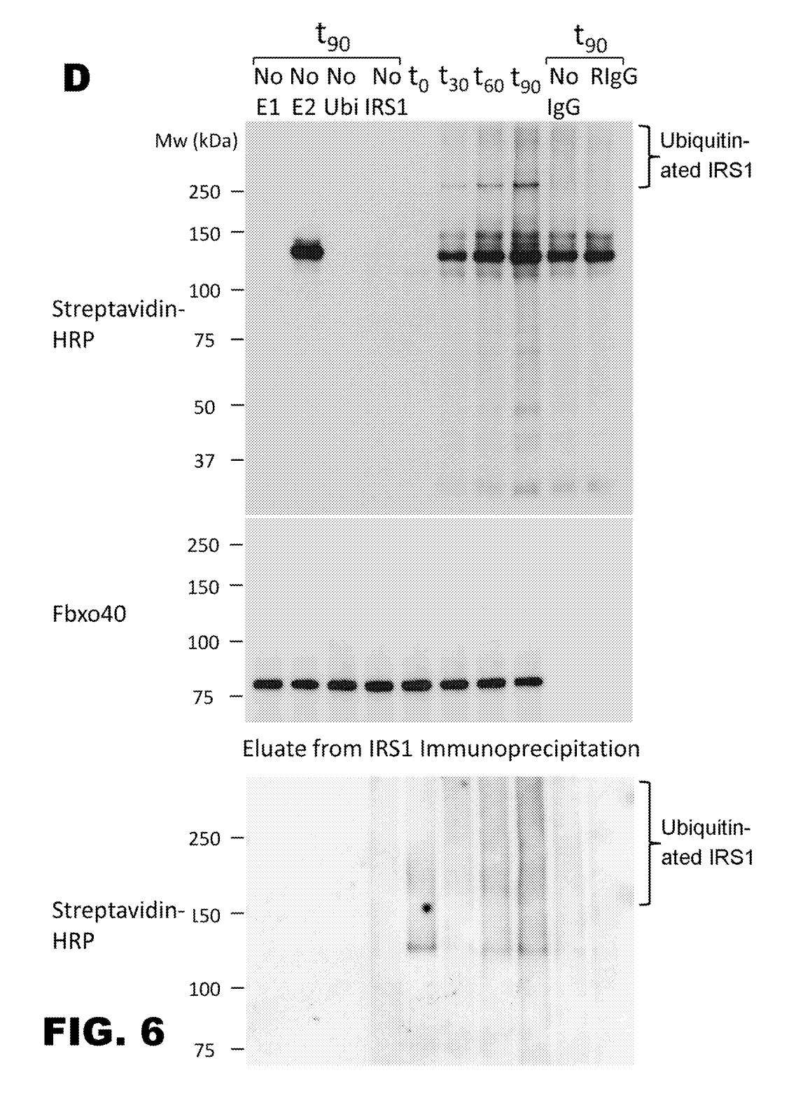

There are around seventy-seven F-box proteins identified so far in mice. We use a functional genomic approach to screen for the F-box proteins targeting IRS1. C2C12 myotubes are transfected with mouse ubiquitin conjugation siRNA library subset 2 from Dharmacon, which contains siRNAs against SOCS-box and F-box containing proteins, or siCON. Forty-eight hours post transfection, cells are treated with 10 nM IGF1 for 16 hours. IRS1 protein level is analyzed by Western blotting. The positive hits from this screen are further corroborated by three different siRNAs from a different source, Qiagen. Knockdown efficiency is evaluated by quantitative real-time PCR for the positive hits. In some cases, all three Qiagen siRNAs cannot silent target gene expression by at least 70% at the mRNA level. In this case, the ON-TARGETplus siRNA SMARTpool from Dharmacon is used for further validation. Out of the 67 F-box protein screened (siRNAs to the remaining genes were not represented in the library), we identify one positive hit, Fbxo40. FIG. 6A demonstrates the dose-dependent rescue of IRS1 by knocking down Fbxo40. Among the three siRNAs targeting Fbxo40, siFbxo40_{—}7 decreases the protein level to 10% of basal level and has the best efficacy, while siFbxo40_{—}9, which leads to the least knockdown, only causes a little increase of IRS1 protein above control level. Importantly, none of these three siRNAs caused an increase in IRS1 protein level without IGF1 treatment (FIG. 6A, left panels).

Fbxo40 is a novel F-box protein that is up-regulated in denervated muscle. Ye et al. 2007 Gene 404: 53-60. We survey the FirstChoice Human Total RNA Panel by quantitative real-time PCR and find that Fbxo40 is highly expressed in heart and muscle (FIG. 3). Furthermore, its mRNA (FIG. 6B) and protein (FIG. 8A) is induced during differentiation of C2C12 cells, which coincides with the accelerated degradation of IRS1 in C2C12 myotubes. IRS1 and Rbx1 can be co-immunoprecipitated with antibody against Fbxo40 (FIG. 6C). Note that Fbxo40 protein level increases after incubation with MG132 (FIG. 6C, compare lanes 1-3), indicating that this protein also rapidly turns over in cells. We also use this immunoprecipitated Fbxo40-Rbx1 complex to ubiquitinate recombinant IRS1 in-vitro and find that IRS1 can be ubiquitinated in a time-dependent manner (FIG. 6D). In this experiment, we include six control reactions. The first four controls are the reactions carried out at 30° C. for 90 min with the omission of E1, or E2 (UbCH5c), or His₆-Biotin-N-terminal Ubiquitin, or recombinant IRS1 respectively. The last two controls are full reactions carried out without immunoprecipitated Fbxo40 (with beads incubated with lysate without IgG or with nonspecific rabbit IgG). Poly-ubiquitinated IRS1 can be observed in full reactions after 60 min or 90 min at 30° C.

These data support the hypothesis that Fbox40 is the E3 ligase that regulates IRS1 protein upon IGF1 treatment.

### Example 5

### Partial Knockdown of Rbx1 Potentiates the Hypertrophic Action of IGF1 in C2C12 Myotubes

As indirect proof that inhibition of Fbxo40 can lead to muscle hypertrophy, we partially knock down Rbx1, which is associated with Fbxo40 and whose function in ubiquinating IRS1 requires Fbxo40.

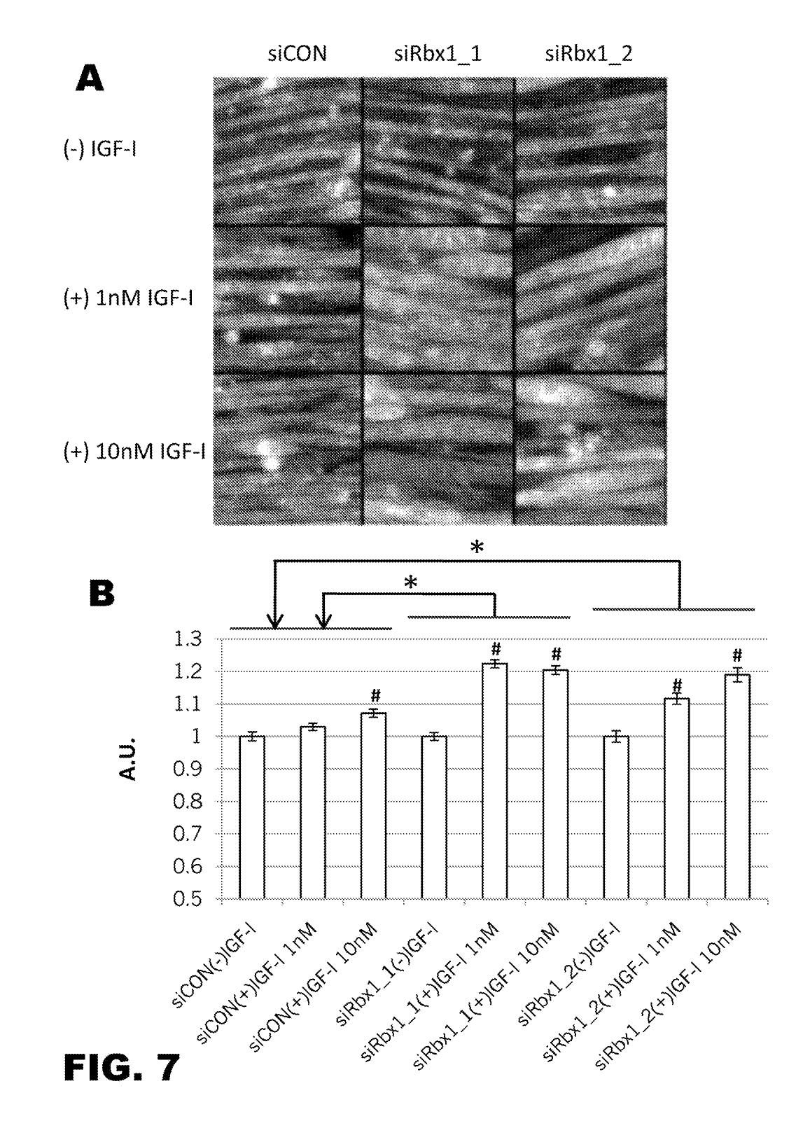

In the following set of experiments, we knock down Rbx1 expression with the siRNAs from Qiagen. Note that as shown in FIG. 5A, siRbx1_{—}1 and siRbx1_{—}2 can lower the protein expression to around 50% of the basal level while siRbx1_{—}3 barely works. We only used siRbx1_{—}1 and siRbx1_{—}2 for this experiment. Transfected cells are treated with two different doses of IGF1 for 24 hours. The diameter of myotubes is measured using automated software with 10 pictures of each treatment group. Knockdown of Rbx1 results in the generation of bigger myotubes even with 1 nM IGF1 treatment (FIG. 7A). In the siCON transfected cells, only 10 nM IGF1 produced statistically significant thicker myotubes. Statistical analysis using two-way ANOVA indicates that the introduction of siRbx1_{—}1 and siRbx1_{—}2 significantly potentiate the hypertrophic action of IGF1 than siCON transfected cells (FIG. 7B).

### Example 6

### Knockdown of Fbxo40 Results in Thicker Myotubes and Increased Muscle Mass

This example shows that knockdown of Fbxo40 results in thicker myotubes, indicating increased muscle mass.

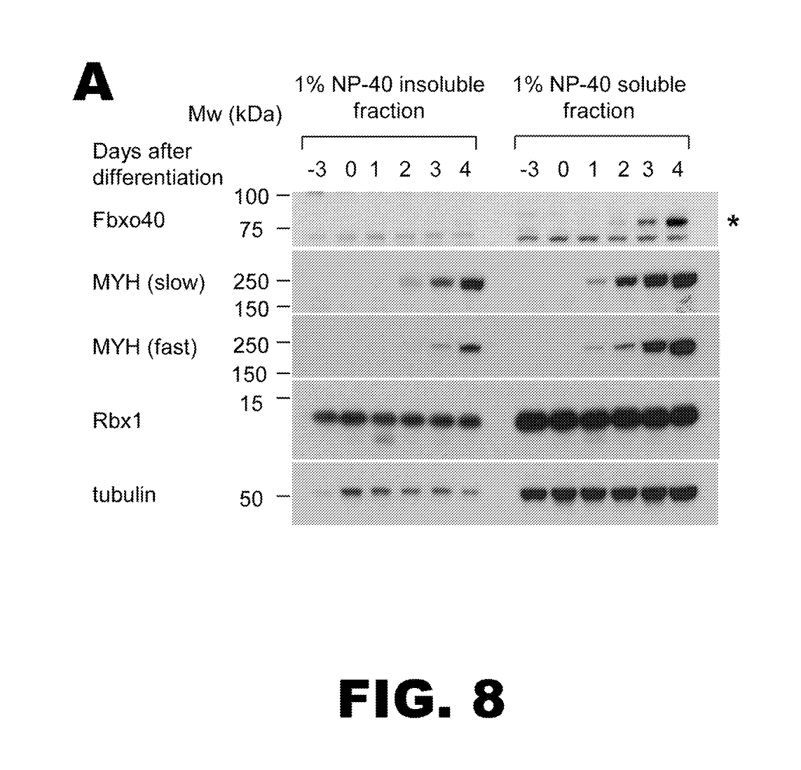

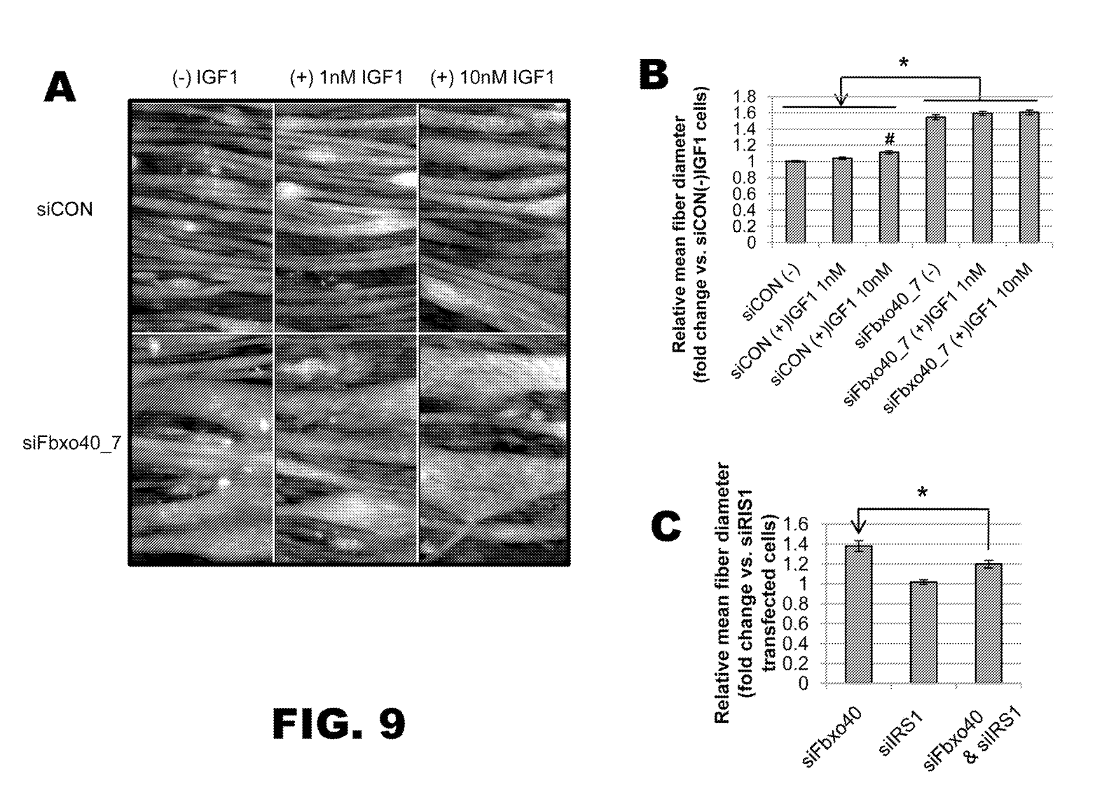

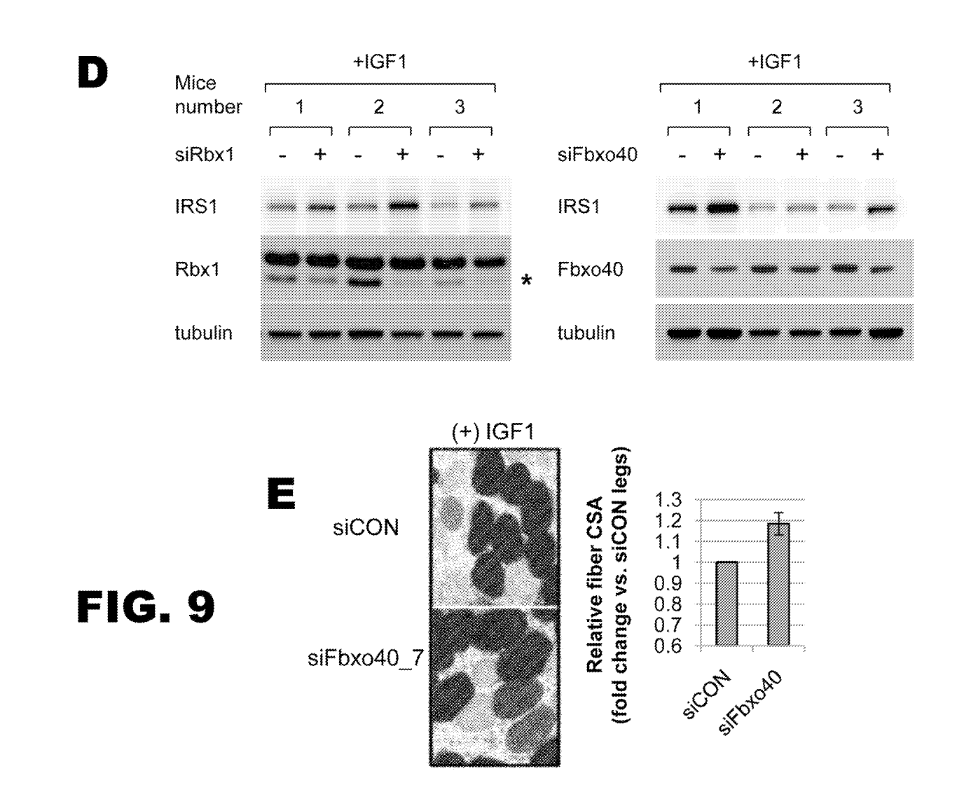

This example is designed to determine the in vivo consequences of Fbxo40 knockdown, since IGF1 is capable of increasing the size of post-differentiated myotubes. Rommel et al. 2001 Nat. Cell Biol. 3: 1009-1013; Jacqueline et al. 2004 Exp. Cell Res. 299: 148-158; and Semsarian et al. 1999 Nature 400: 576-581. Based on the knockdown efficiency of three siRNAs targeting Fbxo40 shown in FIG. 6A, we use the two most effective siRNAs, siFbxo40_{—}7 and siFbxo40_{—}8, for this experiment. Since Fbxo40 expression is detectable at later stages of differentiation (FIGS. 6B and 8A, myotubes are transfected with siCON, siFbxo40_{—}7 and siFbxo40_{—}8 two days post-differentiation; then a day later (day 3 post differentiation), the myotubes are treated with either of two different dose of IGF1 for 24 hours. The diameter of myotubes is measured using automated software, with 10 pictures taken of each treatment group. Knockdown of Fbxo40 results in the generation of dramatically larger myotubes even without additional IGF1 treatment (FIG. 9A). It should be noted that myotubes produce endogenous IGF1 (data not shown). A representative picture of myotubes post-knockdown with siFbxo40_{—}7 is presented in FIG. 9A. Larger myotubes are also observed with siFbxo40_{—}8, the less efficacious siRNA, twenty-four hours post transfection (data not shown). The quantification of myotube diameter is shown in FIG. 9B. Among the siCON transfected groups, only 10 nM IGF1 produce statistically significant thicker myotubes compared to cells treated just with carrier; these show around 11% increase in myotube diameter. However, introduction of siFbxo40_{—}7 results in significant thicker myotubes than siCON transfected cells. The effect seen is quite large—an approximately 50% increase in myotube diameter. Furthermore, when IRS1 is knocked down (mRNA decreased to 65% of siCON transfected cells, data not shown) together with Fbxo40, we only saw an approximately 20% increase in myotube diameter (FIG. 9C), which further proved that Fbxo40 regulates myotube diameter through regulation of IRS1, as opposed to some other potential substrate. These experiments show that knocking down Fbxo40 results in thicker myotubes, indicating increased muscle mass.

We then check whether Fbxo40 also regulates IRS1 protein levels in mice. Mice tibialis anterior muscle is electroporated with siCON in the left leg and siRbx1_{—}1 or siFbxo40_{—}7 in the right leg. A pCMV-LacZ plasmid is co-injected with siRNA to help identify fibers with siRNA expression. After recovery for two days, IGF1 (100 μg per injection) is injected intramuscularly into the tibialis anterior muscle on the 2^{nd}, 5^{th}, and 7^{th} day. Muscle is collected on the 8^{th} day and protein extracts are prepared. FIG. 9D shows that IRS1 protein is higher in siRbx1 and siFbxo40 electroporated samples than siCON samples. In addition, larger muscle fibers are also observed with Fbxo40 knockdown—18%±5% increase compared to siCON electroporated contralateral legs (FIG. 9E).

Thus, experiments are done to determine what the phenotypic consequence is of knocking down Fbxo40 in myotubes. We test this with and without IGF1 stimulation, asking if a decrease in Fbxo40 expression would enhance IGF1-mediated hypertrophy. To our surprise, knockdown of Fbxo40 is sufficient to cause a dramatic increase in myotube size; Fbxo40 knockdown results in a 50% increase in myotube size. This leaves no room for an added effect on top of IGF1. IGF1 (10 nM) alone can only cause an 11% increase in myotube size in siCON transfected cells. However, it should be noted that myotubes produce endogenous IGF1. Therefore, the implication is that the decrease in Fbxo40 results in an uncontrolled autocrine-mediated hypertrophic response to endogenously-produced growth factor, helping to explain why Fbxo40 is necessary to regulate this autocrine signaling. This is proved by knocking down of IRS1 on top of decreased Fbxo40.

Thus the data show that knockdown of Fbxo40 results in thicker myotubes and increased muscle mass.

### Example 7

### Materials and Methods

Materials and methods known to persons of ordinary skill in the art can be used in making and using the present disclosure. The following are example and non-limiting materials and methods, and include those used in Examples 1 to 6.

### Antibodies and Inhibitors

In this study, we use Akt, phospho-Akt (Ser473), phospho-Akt (Thr308), phospho-GSK-3α/β(Ser21/9), IGF1 Receptor β, phospho-IGF1 Receptor β (Tyr1135/1136), rabbit polyclonal IRS1, p44/p42 MAP kinase, phospho-p44/p42 MAP kinase (Thr202/Tyr204), phospho-mTOR (Ser2448), mTOR, phospho-p70 S6 Kinase (Thr389), p70 S6 Kinase, c-Cbl, NEDD4 and α/β-tubulin antibodies from Cell Signaling Technology (Danvers, Mass.); an anti-Cbl-b mouse monoclonal antibody from Santa Cruz Biotechnology, Inc. (Santa Cruz, Calif.); anti-Skp1 antibody from BD Tranduction Laboratories (San Jose, Calif.); anti-Cullin7 and a rabbit polyclonal anti-IRS1 antibody and a mouse monoclonal anti-IRS1 antibody from Millipore (Billerica, Mass.); a rabbit polyclonal anti-Rbx1 antibody from Abcam (Cambridge, Mass.); mouse monoclonal anti-ubiquitin and anti-myc antibodies from Invitrogen (Carlsbad, Calif.). A rabbit polyclonal antibody against Fbxo40 (Fbxo40-691:710: CEKARESLVSTFRARPRGRHF (SEQ ID NO: 34)) has been raised and affinity-purified by Open Biosystems (Huntsville, Ala.).

The signaling inhibitors, rapamycin (used at final concentration of 100 nM), LY294002 (50 μM final concentration), wortmannin (100 nM final concentration), Akt inhibitor API-2 (1 μM final concentration), GSK3 inhibitor LiCl (20 mM final concentration), p38 MAP kinase inhibitor SB202190 (10 μM final concentration), MEK inhibitor PD98059 (25 μM final concentration), MEK1/2 inhibitor (a cell-permeable selective inhibitor of MEK1 and MEK2, 10 μM final concentration) and JNK inhibitor V (20 μM final concentration) are purchased from EMD Chemicals, Inc. (Gibbstown, N.J.).

### Cell Culture

C2C12 myoblasts (ATCC) are cultured and differentiated as described previously. Rommel et al. 1999 Science 286: 1738-1741. Day 0 is defined as the day that cells are changed into low-serum differentiation medium.

### Inhibitors Treatment and Western Blotting

Cells are washed twice with warm serum free DMEM and starved in serum free DMEM for 4 hours with an exchange of fresh medium in the middle. Then the signaling inhibitors are added to pretreat cells for 30 min before incubation with freshly prepared inhibitors with 10 nM IGF1 (the R3 form, Sigma, St. Louis, Mo.) or 10 μM Emetin (Sigma, St. Louis, Mo.) or 20 μM MG132 (Alexis Biochemicals, Carlsbad, Calif.) as specified for an additional 5 hours. Cells are rinsed two times with ice-cold PBS and harvested in ice-cold RIPA buffer with 10 μM MG132 and protease/phosphatase inhibitors tablets (Roche, Basel, Switzerland). Cell lysates are rotated at 4° C. for at least one hour, and spun for 10 min at 16,000×g in a micro centrifuge at 4° C. Protein concentrations of the supernatants are determined by BCA Protein Assay Kit (Pierce/Thermo Fisher Scientific, Rockford, Ill.). Equal amounts of proteins (10-20 μg) are loaded per lane on 4-12% Bis-Tris gels (Invitrogen, Carlsbad, Calif.) and transferred onto PVDF membranes (Millipore, Billerica, Mass.). Membranes are blocked in 10% (w/v) milk in TBST for 1 hour at room temperature, followed by incubation with primary antibody in TBST with 5% BSA at 4° C. for overnight. Secondary antibodies are incubated with membranes at room temperature for 1 hour. Immunoreactivity is detected by ECL and exposed to film or Kodak Image Station 4000R (Eastman Kodak Company, Rochester, N.Y.). Densitometry is analyzed using Kodak Molecular Imaging software, version 4.0.

### Measurement of Protein Stability

Cells are washed twice with warm serum-free DMEM and starved in serum-free DMEM for 4 hours with an exchange of fresh medium in the middle. At time zero, 10 μM Emetin, or 100 μg/ml of cycloheximide, or 10 nM IGF1, or 20 μM MG132 are added to cells as specified. At the indicated time intervals, cells are rinsed twice with PBS and harvested in ice-cold RIPA buffer with 10 μM MG132 and protease/phosphatase inhibitors. Cell lysates are rotated at 4° C. for at least one hour, and spun for 10 min at 16,000×g in a micro centrifuge at 4° C. The supernatants are analyzed by western blotting.

### In-Vivo Ubiquitination Assay and Immunoprecipitation

A His-myc-ubiquitin adenoviral expression constructs are sent to Welgen Inc. (Worcester, Mass.) for production and purification. The purified adenovirus is added to C2C12 cells 2 days postdifferentiation at a concentration of 2×10⁸ pfu/ml for overnight. Forty-eight hours after infection, cells are starved and treated with 10 nM IGF1, or 20 μM MG132, or 5 μM G5 (an isopeptidase inhibitor, EMD Chemicals, Inc., Gibbstown, N.J.) as specified for 5 hours. Then cells are rinsed two times with PBS and harvested in 800 μl ice-cold RIPA buffer per 10 cm dish with 10 μM MG132, 5 μM G5 and protease/phosphatase inhibitors tablets. Cell lysates are rotated at 4° C. for at least one hour, and spun for 10 min at 16,000×g in a micro centrifuge at 4° C. Protein concentration in the supernatant is determined by BCA analysis. This material (1 mg of total protein) is incubated with the rabbit polyclonal IRS1 antibody (Cell Signaling, Denvers, Mass.) and nonspecific rabbit IgG (each 3 ug) along with 40 μl of M-280 sheep anti-rabbit IgG Dynabeads (Invitrogen, Carlsbad, Calif.) for overnight at 4° C. with rotating. The beads are then washed three times with Triton Lysis Buffer (50 mM Tris-HCl, 150 mM NaCl, 5 mM EDTA and 1% Triton, pH7.4). Elution is carried out by resuspending beads in 30 μl of sample buffer with 100 mM DTT, then boiling at 95° C. for 5 min. An aliquot (20 μg) of the lysate is analyzed by Western blotting along with 100% of the eluate.

In the IRS1 and Rbx1 Co-Immunoprecipitation experiment, day 3 C2C12 myotubes are starved in serum free DMEM for 4 hours with an exchange of fresh medium in the middle. Then cells are treated with 10 n M IGF1 along with 20 μM MG132 in serum-free DMEM for 16 hours. Cells are rinsed two times with ice-cold PBS and lysed in ice-cold Triton Lysis Buffer supplemented with 10 μM MG132 and protease/phosphatase inhibitors. Cell lysates are rotated at 4° C. for at least one hour. The 16,000×g supernatants (1 mg) are incubated with the rabbit polyclonal IRS1 antibody (Cell Signaling) and nonspecific rabbit IgG (each 3 ug) along with 40 μl of M-280 sheep anti-rabbit IgG Dynabeads for overnight at 4° C. with rotating. The bound proteins are eluted with 30 μl of sample buffer with 100 mM DTT.

Alternatively, in the experiment of IRS1 and Rbx1 Co-Immunoprecipitation with Fbxo40, day 3 C2C12 myotubes are starved and treated with 10 nM IGF1 or 20 μM MG132 as described above. Cell lysate (1 mg) are incubated with the affinity-purified Fbxo40 antibody and non-specific rabbit IgG (each 3 ug) along with 40 μl of M-280 sheep anti-rabbit IgG Dynabeads.

### In-Vitro Ubiquitination Assay

For the in vitro ubiquitination assay, yeast E1, human recombinant UbCH5c, human His₆-Biotin-N-terminal Ubiquitin, Ubiquitin Aldehyde, and Energy Regeration Solution (ERS) are purchased form Boston Biochem, Inc. (Cambridge, Mass.). Lactacystin is purchased from Alexis Biochemicals (San Diego, Calif.). Purified recombinant IRS1 protein is purchase from Millipore (Billerica, Mass.).

Day 2 C2C12 myotubes are treated with 10 nM IGF1 and 20 μM MG132 in complete medium for 16 hours. Cells are rinsed two times with ice-cold PBS and lysed in ice-cold Triton Lysis Buffer supplemented with 10 μM MG132, 5 μM G5 and protease/phosphatase inhibitors. Cell lysate is rotated at 4° C. for at least one hour. The 16,000×g supernatants (1 mg) are incubated with anti-Fbxo40 antibody or nonspecific rabbit IgG (each 3 ug) along with 40 μl of M-280 sheep anti-rabbit IgG Dynabeads for overnight at 4° C. with rotating. In one control condition, the lysate without any IgG is incubated with the beads. Then the beads are washed three times with Triton Lysis Buffer for 10 minutes with rocking. The ubiquitination reaction is carried out by addition of E1 (125 nM), UbCH5c (3.125 μM), His₆-Biotin-N-terminal Ubiquitin (5.4 μM), 1×ERS, Ubiquitin Aldehyde (5 μM), Lactcystin (20 μM), recombinant IRS1 (40 ng), and reaction buffer (50 mM HEPES, 0.6 mM DTT, pH 7.4) in a total volume of 40 μl to the beads. Reactions are carried out at 30° C. for 0, 30, 60, 90 minutes. Control reactions are carried out with the omission of E1, or E2 (UbCH5c), or His₆-Biotin-N-terminal Ubiquitin, or IRS1 at 30° C. for 90 minutes. Each condition is carried out in duplicate sets. For one set, the reaction is ended by separating the reaction mix from the beads, adding SDS-PAGE sample buffer, and heating. For another set, additional ice-cold Triton Lysis Buffer (800 μl) is added to stop the reaction. Then the reaction mix is separated from the beads and subjected to another round of immunoprecipitation with rabbit polyclonal anti-IRS1 antibody (Cell Signaling) along with 40 μl of M-280 sheep anti-rabbit IgG Dynabeads for overnight at 4° C. with rotating. The bound proteins are eluted with 35 μl of sample buffer with 100 mM DTT. Reactions are run on 4%-12% Bis-Tris gels with MOPS running buffer or 3%-8% Tris-Acetate gels (Invitrogen) and transferred to PVDF membranes for immunoblotting with streptavidin-conjugated horseradish peroxidase (Pierce).

siRNA Sequences

Unless indicated otherwise, siRNAs are purchased from Qiagen (Valencia, Calif.). We used AllStars Negative Control siRNA as our control siRNA. The target sequences of siRNAs are:

| | | | SEQ |
|---|---|---|---|
| Gene | siRNA | | ID |
| Name | Name | Target Sequence | NO. |
| Cbl-b | siCbl-b_1 | AAGCATTTATTTGTCAATAAA | 3 |
| | siCbl-b_2 | CAGGAGTGTCATAATGCTGTA | 4 |
| | siCbl-b_3 | CTCACGTATGATGAAGTTAAA | 5 |
| | siCbl-b_4 | CTCCATCATATTATTTGTGTA | 6 |
| c-Cbl | siC-Cbl_1 | CCGCACTGTCTTGTCAAGATA | 7 |
| | siC-Cbl_2 | CTGGTCCTCTTTGCTCAGAAA | 8 |
| Cullin1 | siCullin1_1 | CACGTGTAATCTGCTATGAAA | 9 |
| | siCullin1_2 | AAGCATGATCTCCAAGTTAAA | 10 |
| | siCullin1_3 | CAGGGTCTGCATGACCCACAA | 11 |
| Cullin2 | siCullin2_1 | CAGCGCTGATTTGAACAATAA | 12 |
| | siCullin2_2 | AACCAGAGTATTTATATCTAA | 13 |
| | siCullin2_3 | TCCAAATTTATAAATATTAAA | 14 |
| | siCullin2_4 | ACGGGAGAATATTACAAGCAA | 15 |
| Cullin7 | siCullin7_1 | CAGCATCAAGTCCGTTAATAA | 16 |
| | siCullin7_2 | GAGGATGTGATTGATATTGAA | 17 |
| | siCullin7_3 | ATGCTGGAATTTGAAGCTAAA | 18 |
| Fbxo40 | siFbxo40_7 | CACCTCCTGGAAAGTCCACAA | 19 |
| | siFbxo40_8 | GTGGGAAAGTATGTTCAGCAA | 20 |
| | siFbxo40_9 | AGCCGTGGATGCCAAAGACTA | 21 |
| Fbxw8 | siFbxw8_1 | CAGGTGTTTGCCAAACCTCAA | 22 |
| | siFbxw8_2 | CTGGTTGACTACCTTGAAATA | 23 |
| | siFbxw8_3 | TACGAACTGGCCATCAATATA | 24 |
| NEDD4 | siNEDD4_1 | CAGACTGACATTCCAAACAAA | 25 |
| | siNEDD4_2 | TTGGACAAAGAAAGTCTTTAA | 26 |
| | siNEDD4_3 | ATCCAAGAAGTCACAAATCAA | 27 |
| Rbx1 | siRbx1_1 | AAGAAGCGCTTTGAAGTTAAA | 28 |
| | siRbx1_2 | CTGGGACATTGTGGTTGATAA | 29 |
| | siRbx1_3 | ATGCAGCAATCTCTAGAACAA | 30 |
| Skp1 | siSkp1_1 | CAAAGTTGACATTGTAAATAA | 31 |
| | siSkp1_2 | AACATGATACTTGTTCATGAA | 32 |
| | siSkp1_3 | AACATTAAAGTGTATGGTAAA | 33 |

The siRNA library targeting proteins involved in ubiquitin conjugation is purchased from Dharmacon (Dharmacon/Thermo Fisher Scientific, Lafayette, Colo.). This library contains three subsets, which include E1, E2, F-box and SOCS box proteins, cullins, HECT domain containing, RING finger and RING finger-like domain containing E3s. Each gene is targeted by a pool of four individual siRNAs. The ON-TARGETplus Non-Targeting siRNA pool from Dharmacon is used as a negative control. Only one strand of the double-stranded RNA is shown. Fbxo40 siRNA siFbxo40_{—}7 targets the sequence CACCTCCTGGAAAGTCCACAA (SEQ ID NO: 19); this siRNA is Qiagen catalog no. S104390162. Fbxo40 siRNA siFbxo40_{—}8 targets the sequence GTGGGAAAGTATGTTCAGCAA (SEQ ID NO: 20); this siRNA is Qiagen catalog no. S104390169 Fbxo40 siRNA siFbxo40_{—}9 targets the sequence AGCCGTGGATGCCAAAGACTA (SEQ ID NO: 21); this siRNA is Qiagen catalog no. S104390176.

### RNAi

Day 1 C2C12 cells are transfected with siRNAs as described in Qiagen protocol for HiPerfect transfection reagent. Briefly, the procedure is described below for cells grown in E-well plates. First, 56 μl of OPTI-MEM (Invitrogen) is mixed with 4 μl of siRNA (10 μM stock). Then 40 μl of HiPerFect transfection reagent is added to this mix to give a total volume of 100 μl. The mix is incubated for 10 minutes at room temperature to allow the formation of transfection complex. In the mean time, the cells are fed with fresh 2 ml differentiation medium per well of 6-well dish. The 100 μl complexes are added drop-wise onto the cells in one well. The plates are gently swirled to ensure uniform distribution and returned to 37° C. incubator. Forty-eight hours after transfection, cells are starved for 4 hours in serum free DMEM and treated with 10 nM IGF1 or 20 μM MG132 for 16 hours at 37° C. Then, cells are lysed and proteins are analyzed by western blotting as described in the section of Inhibitors Treatment and Western Blotting. The RNA expression is also checked 48 hours post transfection.

Day 1 C2C12 cells grown in 12-well plates are transfected with the Dharmacon siRNA library (10 μM stock solution) using Dharmafect 3 according to the protocol described by Dharmacon. The final concentration of siRNAs incubated with cells is 100 nM. Cells are treated and RNAs is harvested 48 hours post transfection as described above.

### Quantitative Real-Time PCR

Total RNA is isolated from cells using Tri reagent (Molecular Research Center, Inc., Cincinnati, Ohio). For each treatment condition, there are three independent samples.

Genomic DNA is removed from RNA samples with the help of TURBO DNA-free kit (Applied Biosystems/Ambion, Austin, Tex.). RNA samples (1 μg of each sample) are reversed transcribed to cDNA using High Capacity cDNA Reverse Transcription kit (Applied Biosystems, Foster City, Calif.). The resulting cDNA samples are diluted 1:10 and 9 μl are analyzed in triplicate in 384-well plates with Taqman Gene Expression Assays listed in the table below using the 7900HT Fast Real-Time PCR System (Applied Biosystems). Data are analyzed with SDS software v2.2. The mRNA levels in the control siRNA transfected or carrier treated cells are set at 1. The relative fold change is calculated using the 2^{-ΔΔCt} method for each sample. The 5728mean fold change and standard error of the means of three independent samples are plotted.

| Gene Name | Assay ID |
|---|---|
| human Fbxo40 | Hs00212488_m1 |
| human β-actin | 4333762T |
| mouse B2M | Mm00437762_m1 |
| mouse Cullin1 | Mm00516318_m1 |
| mouse Cullin2 | Mm00518586_m1 |
| mouse Fbxo40 | Mm01343539_m1 |
| mouse Fbxw8 | Mm00554876_m1 |
| mouse GAPDH | 4352339E |
| mouse IRS1 | Mm01278327_m1 |
| mouse β-actin | 4352341E |

FirstChoice Human Total RNA Survey Panel is purchased from Ambion (Applied Biosystems/Ambion, Austin, Tex.).

### Measurement of Myotube Diameter

Cells are washed twice with ice-cold PBS after treatment and then fixed with 5% Glutaraldehyde at 37° C. for 15-30 min followed by two rinses with PBS. Ten pictures are taken for each condition with a ten-fold magnification lens using a Carl Zeiss Axio Observer.Z1 fluorescent microscope (Carl Zeiss Inc., thornwood, NY). Myotube diameters are determined from the TIFF format pictures using an automatic software developed by Novartis. The mean value of untreated control cells in each transfection group is set at 1 to eliminate the complications that may result from the introduction of specific siRNA. The relative mean fold change and standard error of the means are calculated and plotted.

### Statistical Analyses

Except in FIGS. 7B and 9B, differences between two treatment conditions are analyzed using one-way ANOVA. Significance is determined by Bonferroni post test post hoc test. Values are considered significant at p<0.01.

In FIGS. 7B and 9B, differences between groups are analyzed using two-way ANOVA. Significance is determined by Bonferroni post test. Values are considered significant at p<0.01.

### Mice Procedures

C57BL/6 J mice (Taconic Inc, Hudson, N.Y.) at 12-14 weeks of age are anesthetized with 2-3% isofluorane. Hair is shaved from area surrounding muscle. On day 0, 500 pmole of siCON along with 50 μg of pCMV-LacZ plasmid is injected into the left tibialis muscle. The right tibialis muscle is injected with 500 pmole siRbx1_{—}1 or siFbxo40_{—}7 and 50 μg of pCMV-LacZ plasmid. Immediately following injection, limbs are pulsed with 5 “positive” pulses of 125V/cm, 30 ms duration, with 400 ms interval time followed by 5 “negative” pulses of the same parameter. Mice are allowed to recover in their cage for 2 days. Long-R3-IGF1 (100 μg per injection) is injected intramuscularly into the tibialis muscle on the days 2, 5, 7. On day 8, mice are sacrificed and tibialis muscle is rapidly dissected and snap frozen in 2-methylbutane pre-cooled in liquid nitrogen. Serial transverse sections, 8 μm thick, are cryosectioned and subbed to positively charged slides. Sections are stained for i-galactosidase (LacZ). Images of the entire tissue section are acquired using Imagescope (Aperio) and the cross-sectional area (CSA) of LacZ positive fibers is measured using Adobe Photoshop. The rest of tibialis muscle is pulverized under liquid nitrogen and protein is extracted. All animal procedures are approved by the Institutional Animal Care and Use Committee of Novartis Institute for Biomedical Research and are in compliance with the Animal Welfare Act Regulations 9 CFR Parts 1, 2 and 3, and US regulations (Guidelines for the Care and Use of Laboraory Animals, 1995).

We claim:

1. A method of screening compositions for the ability to increase muscle mass or prevent, limit or reduce the loss of muscle mass in an individual, comprising:
(a) ascertaining the level or activity of Fbxo40 in a cell from the individual,
(b) optionally, treating the cell with a composition comprising an antagonist to Fbxo40, and
(c) optionally, ascertaining the level or activity of Fbxo40 in the cell again,
wherein an elevated level of Fbxo40 relative to a control is an indication that the subject has or is at risk of developing a muscle-wasting disorder, and wherein an ability of the composition to decrease the level or activity of Fbxo40 is correlated to the ability to increase muscle mass or prevent the loss of muscle mass in an individual.

2. The method of claim 1, wherein the individual is afflicted with a muscle wasting-associated selected from: cachexia, cancer, tumor-induced weight loss, sepsis, chronic heart failure, rheumatoid arthritis, acquired immune deficiency syndrome, sarcopenia, diabetes, hypertension, high levels of serum cholesterol, high levels of triglycerides, Parkinson's disease, insomnia, drug addiction, pain, insomnia, hypoglycemia, compromised liver function, cirrhosis, gall bladder disorders, chorea, dyskinesia, kidney disorder, and/or uremia.

3. The method of claim 1, wherein the antagonist reduces the level, expression or activity of Fbxo40.

4. A method of diagnosing or monitoring the level of muscle mass increase or maintenance, or reduced loss in an individual, comprising:
(a) ascertaining the level or activity of Fbxo40 in a cell from the individual,
(b) optionally, treating the cell with a composition comprising an antagonist to Fbxo40, and
(c) optionally, ascertaining the level or activity of Fbxo40 in the cell again,
wherein an elevated level of Fbxo40 relative to a control is an indication that the subject has or is at risk of developing a muscle-wasting disorder, and wherein an ability of the composition to decrease the level or activity of Fbxo40 is correlated to the ability to increase muscle mass or prevent the loss of muscle mass in an individual.

5. The method of claim 4, wherein the individual is afflicted with a muscle wasting-associated disorder selected from: cachexia, cancer, tumor-induced weight loss, sepsis, chronic heart failure, rheumatoid arthritis, acquired immune deficiency syndrome, sarcopenia, diabetes, hypertension, high levels of serum cholesterol, high levels of triglycerides, Parkinson's disease, insomnia, drug addiction, pain, insomnia, hypoglycemia, compromised liver function, cirrhosis, gall bladder disorders, chorea, dyskinesia, kidney disorder, and/or uremia.

6. The method of claim 4, wherein the antagonist reduces the level, expression or activity of Fbxo40.

7. A method of increasing muscle mass or maintaining or preventing the loss of muscle mass in an individual, comprising administering to the individual a therapeutically effective amount of an antagonist of Fbxo40.

8. The method of claim 7, wherein the individual is afflicted with a muscle wasting-associated disorder selected from: cachexia, cancer, tumor-induced weight loss, sepsis, chronic heart failure, rheumatoid arthritis, acquired immune deficiency syndrome, sarcopenia, diabetes, hypertension, high levels of serum cholesterol, high levels of triglycerides, Parkinson's disease, insomnia, drug addiction, pain, insomnia, hypoglycemia, compromised liver function, cirrhosis, gall bladder disorders, chorea, dyskinesia, kidney disorder, and/or uremia.

9. The method of claim 7, wherein the method further comprises administering physiotherapy, nutrients, electrical stimulation, electrical neuromuscular stimulators of NMES, neural input to the muscles; and/or one or more of the following: steroid, hormone, growth hormone, growth hormone secretagogue; ibutamoren mesylate (MK-677), gingko biloba extract, flavoneglycoside, ginkgolide, amino acid supplement, leucine, amino acid precursor, leucine precursor, pyruvate and pyruvate metabolite, beta-hydroxy-beta-methylbutyrate, alpha-ketoisocaproate, branched chain amino acid, erythropoietin, opiate, scopolamine, insulin, insulin-like growth factor-1 (IGF1), and/or testosterone; and/or inhibitor of aldosterone, alpha receptor, Angiotensin II, beta receptor, cathepsin B, chymase, endothelin receptor, eukaryotic initiation factor 2-alpha (eIF2-alpha), imidazoline receptor, interferon, MAFbx (Muscle Atrophy F-box), MuRF1 (Muscle RING Finger 1), myostatin, parathyroid hormone related protein (PTHrP) and/or its receptor, proteolysis-inducing factor (PIF), RNA-dependent serine/threonine protein kinase (PKR), tumor necrosis factor alpha (TNF-alpha), and/or xanthine oxidase.

10. The method of claim 7, wherein the antagonist reduces the expression, level, or activity of Fbxo40.

11. The method of claim 7, wherein the antagonist of Fbxo40 is a low molecular weight compound.

12. The method of claim 7, wherein the antagonist is a polypeptide.

13. The method of claim 7, wherein the antagonist of Fbxo40 is a siRNA that binds to a nucleic acid encoding Fbxo40.

14. The method of claim 13, wherein the siRNA is blunt-ended.

15. The method of claim 7, wherein the antagonist of Fbxo40 is an antibody that binds to Fbxo40.

16. A composition comprising an antagonist of Fbxo40, wherein the antagonist reduces the expression, level or activity of Fbxo40 and increases muscle mass or prevents, limits or reduces the loss of muscle mass.

17. The composition of claim 16, wherein the composition further comprises one or more of the following: steroid, hormone, growth hormone, growth hormone secretagogue; ibutamoren mesylate (MK-677), gingko biloba extract, flavoneglycoside, ginkgolide, amino acid supplement, leucine, amino acid precursor, leucine precursor, pyruvate and pyruvate metabolite, beta-hydroxy-beta-methylbutyrate, alpha-ketoisocaproate, branched chain amino acid, erythropoietin, opiate, scopolamine, insulin, insulin-like growth factor-1 (IGF1), and/or testosterone; and/or inhibitor of aldosterone, alpha receptor, Angiotensin II, beta receptor, cathepsin B, chymase, endothelin receptor, eukaryotic initiation factor 2-alpha (eIF2-alpha), imidazoline receptor, interferon, MAFbx (Muscle Atrophy F-box), MuRF1 (Muscle RING Finger 1), myostatin, parathyroid hormone related protein (PTHrP) and/or its receptor, proteolysis-inducing factor (PIF), RNA-dependent serine/threonine protein kinase (PKR), tumor necrosis factor alpha (TNF-alpha), and/or xanthine oxidase.

18. The composition of claim 16, wherein the antagonist reduces the expression, level or activity of Fbxo40.

19. The composition of claim 16, wherein the antagonist of Fbxo40 is a low molecular weight compound.

20. The composition of claim 16, wherein the antagonist is a polypeptide.

21. The composition of claim 16, wherein the antagonist of Fbxo40 is a siRNA that binds to a nucleic acid encoding Fbxo40.

22. The composition of claim 21, wherein the siRNA is blunt-ended.

23. The composition of claim 16, wherein the antagonist of Fbxo40 is an antibody that binds to Fbxo40.


SEQUENCE LISTING (34 sequences)

SEQ ID NO: 1 - protein, 709 aa, Homo sapien
MGKARRSPPGHHRHCEGCFNRHCHIPVEPNTSCLVISCHLLCGATFHMCKEAEHQLLCPLEQVPCLNSEYGCPLSMSRHKLAKHLQVCPASVVCCSMEWNRWPNVDSETTLHENIMKETPSEECLDTALALQDQKVLFRSLKMVELFPETREATEEEPTMNGETSVEEMGGAVGGVDIGLVPHGLSATNGEMAELSQEEREVLAKTKEGMDLVKFGQWENIFSKEHAASALTNSSASCESKNKNDSEKEQISSGHNMVEGEGAPKKKEPQENQKQQDVRTAMETTGLAPWQDGVLERLKTAVDAKDYNMYLVHNGRMLIHFGQMPACTPKERDFVYGKLEAQEVKTVYTFKVPVSYCGKRARLGDAMLSCKPSEHKAVDTSDLGITVEDLPKSDLIKTTLQCALERELKGHVISESRSIDGLFMDFATQTYNFEPEQFSSGTVLADLTAATPGGLHVELHSECVTRRHNKSSSAFTFTCNKFFRRDEFPLHFKNVHTDIQSCLNGWFQHRCPLAYLGCTFVQNHFRPPGQKAKVIYSQELKTFAIKPEVAPELSEGRKNNHLLGHGGKSQNSLTSLPLEILKYIAGFLDSVSLAQLSQVSVLMRNICATLLQERGMVLLQWKKKRYSHGGTSWRVHREIWQFSSLFSKIKSWEFNEVTSMSEHLKSCPFNIVEHKTDPILLTSMCQPREQARESLVSTFRIRPRGRYVS

SEQ ID NO: 2 - DNA, 5722 bp, Homo sapien
atttttaacttcgcaacacttggactatttctgttgaagttttctctcctttccctgccttcccaacagaacgctgtcctctactgcagctgatgcaacccagccacctcccggcaatccgcttacttgcaatcaagggttcaggtgcaagcagatgctgactcagcctgttccattaagagctaagaagcaagaagaaattggggcgccatggggaaagcccgcagatccccgccagggcaccacaggcattgtgagggatgcttcaaccgccactgccacattcctgtggaacccaacacctcctgcctggtaataagctgccacctgctctgtggtgccaccttccacatgtgcaaagaggcagagcaccagctcctctgccctttagagcaggttccgtgcctcaactccgaatatggctgccctctgtccatgtcccgccacaaactggccaagcacctgcaggtgtgccccgccagcgtggtctgctgctccatggagtggaaccgctggccaaatgtggactctgaaaccacccttcatgaaaacatcatgaaagagacccccagtgaggagtgtttggacacagccctggccctgcaggatcagaaggtcctcttcagatccttgaaaatggtggaacttttcccagaaactagagaggctactgaggaggaaccaactatgaatggtgaaaccagtgtggaggaaatgggaggagcagtgggtggagtggatatcggtttggtaccacatggtctgtcagcaactaatggggagatggcagagctaagtcaagaagaacgggaggtgctagccaaaaccaaagaagggatggacctggtcaagtttggccagtgggaaaatattttcagcaaagagcacgcagcctctgctttaacaaattcatcagcgagctgtgagagcaagaacaagaatgactccgagaaagaacagatttccagtggccataacatggtagaaggagagggcgctcccaaaaagaaagaaccacaggaaaatcagaagcagcaggacgttcgtacagccatggaaaccacagggcttgccccttggcaggatggtgttctggaaagactgaaaacagctgtggatgcaaaggactataacatgtatctagtgcacaatgggcggatgctgatacactttggtcagatgcctgcttgtacacccaaggagagagactttgtttatggcaagctggaggctcaggaagttaagactgtttacaccttcaaagttcctgtgagctactgtggaaagcgagctcgacttggagatgccatgttgagttgtaagccaagtgaacacaaggcagtggatacttcagatttggggatcactgtggaggacctgcccaaatcagatctcatcaagaccaccctccagtgtgctttggaaagagaactcaaaggccacgtcatctctgaatccagaagcattgatggactgttcatggattttgccacacaaacatacaactttgagccagaacagttttcctctgggacagtgctggctgacctaaccgctgccaccccagggggactccacgtggagctccacagcgagtgtgtgaccaggagacacaacaaaagcagctctgccttcactttcacttgcaacaaattcttcaggagggatgagttccccctgcacttcaagaatgtccacacagacattcagtcatgtctcaatggctggttccagcatcgatgccccctcgcctacttgggatgtacatttgttcaaaaccatttccgtcccccagggcaaaaggcaaaagtaatctatagccaggagctcaagacctttgccattaagccggaggttgctccagagctgagcgagggaaggaagaacaaccatcttttgggtcatggaggaaaaagccagaattctttaaccagcctgcccctggagattttgaagtacattgctgggttcttggacagcgtcagcctggcccagctctcccaggtgtctgtgctgatgaggaatatctgtgccactttgttacaagagagaggaatggtccttttgcaatggaagaaaaagaggtattcccatggaggcacctcctggagagtccacagagagatctggcagttcagcagcctcttctccaaaatcaagagctgggagtttaatgaagtcacctccatgtctgagcacctgaagtcctgtcctttcaacattgtagagcacaaaactgacccgattcttttgactagcatgtgtcagccccgtgagcaggcccgagagagcttagtctccacctttagaatcagaccacgaggaagatacgtctcctaaaaattcagatgccactcgatgcacccttcttggatttcttctcggagttcctgaagtaggacagagtgtgtggttttgaggactcccttctgtaaactgcctatttgcttatcggggtgtattggaacacgcaatgtccttcgaaacctcaacacgaggcctaagaatttcctaagccatgtcttgtaccatagtgccacattgatgacttgtttccttttttcttttcttttcttttcttttttctttctttctaaaatagattggtctgagaagaaaataagtaatttgaggccatttggaagatgggcccaatttcttaagtgatgagagagcacgaaattccataaccagtacaggcctgtgcttttacatgggctttttagttcacaaagcactttcaaatttatgggacaggaaatgcaggatgggactccccagggaacgcagggtgaagggaacaaagctggaggctctggagctgggtctgttttgacggtcaagtccagggctcattttggttattctactgcctctaggccagggtagtcctaacacagcctgacataggagagcccctggctgagcatggcagccttgaagacaccacaggccaaaacatgaggggcagaaatgggatcacagagtctgttgctagaatcttggcaacatacagcaggaaagccttgataaatcgggagtccaaaggagacaccatatttatggagaacattaggacaaaaagtcaccaacttactttgtaacattttaataatgacttaagggtcaagatttttttcttctgaaaattatgttctgagttaggcagaacatagccatggccctgggccaccctgtgctatctgaaatgacctcaatacactaatgccaacctcagcgtcatgccagaatgcacagggcagcccagggagatcacacctttggcaaagtccagacaaggcccactgcagttcctatggcgccagtcaccagctcctagacagcacttgggtaccccattggggtcttggagaggaagacatgtgaacataacggctccctgaaattgctcctacccatccatatttctggtcatgctttcagtctgacaaaaatggatgatactgctgtttttggtaacaaacagtgaatattcataagaacaaaagtaaaagaaaaaaagacacagtagaaactggcatcccctaaagcagggcttcttagccttggaactattgacatttttaaatggataattctttttttttttttctaggtggggaggggatggagttcactcttgttgcccaggctggagcgcaatgacatgatctcggctcaccgcaacctccgcctcctgggttcaagcgattctcctgcctcagcctcccgagtagctgggattactcgcctggctaattttgtatttttagtagagacgggctttctccatgttgttcaggctggtctcaaactcccgacctcaggtgactcgcccgccttggccttccaaagtgctgggtttacaggtgtgagccactgcgccctgcctgaactggataattctttgttgcaagggactgttctgtgtactacaggatacttggcagcatccttggcctatccattaaatgtcagtagcacccccacagtggcaacaatcaaaaatgtcaccagacattgctaaatattggggagcaaaatggctccccgttgaaaatccctaaaggatgtcatactagtgacaataagttaggatatgcttattttttagtacagcaaaatcttatcgcacatagctatccacaatagttatgatttaatgcagctctttatttatgaaataggttttagacatgtggtgattttaagttgggaaccagaaggaaatgattcgtttggtatggcttcatgtccttcagccacccccaagaatgtatcctttcagctctctttggttatacctgaagccaggagcgttgagttattagccttgtgtttatattcctctcactgtaattggtgtcattttcccagcagtcctagcagtcctcaagcaagtgggaaatcggaaaagaaaaggacaggcattgtagggaagcagaggataaagaatttagccaacaaaagaaacaatctagtcaatctgggtgcttttatttcctgggttctctctaaacatggctcagagctggtgtagatgaagtaggtgaaacctctgaaaagagtctagaaggcagtagagcaagtcccagaccagaaacatgctcatcttttcatcgtaatgtgccactcggtactatttggtaatgtcactctatttttcctaatcccatcctttggtttgtatttcatatttgtatataaggcaccattttctaaaaatatgactagggtgtgacctaaggttttattctgtgaagatgagtaactggaaagaagctaacactgcagtgggaaggaaggaagagagttgtccaggtggtagttcgacgtgttttgaatctagtccttcctacatggaggataaaagctcctaaagtccactctgggtttgtgattttaatagaaatagaaagggaaactatagaccaatggagatgaaaatcaggggctatcgacagatggaggagaaataaggtgctacatagagaaaggaagagggcagaaggctttcccttcccaaactgggtgagctggggaagccttggttcaggagagtggcactgcccacaactgctttgtgggttgtgcacttccagccgcactctccccctccagttgctgccttcagagccgtactgaagcacgagcttcaataagacaagcacacttcatagtgagagggcagcggtaccaaagcctttcagagagactatggattagacagaaatgatttgtgagaggaagctggagtgaacagcatgaacagcgagtgttacctgacagaggcaagacagctagaagtggcttcagatttagaaacagctgaggggagcaaagacggactgtgtacacagggagggaggatgtctatgggcagagcccttggtgagtatcatcaccaagaaaggcagtccagagtagagatcagccgaatatggaggctgaggtctgtagaactgggccagagaggaccttactgccttagtagcataagggtctggaaaagaagtttctatctcacaacaaaggaaaaagtgaaaagcaaggtggaacttgaagatacgtcacgaaaatcactataaaagtctgatttatgtgtgatgtcaaatcaaactgaaatgaagaatgagattgagtatatctgtggtgactgacctctgtatactagaaacctcaacatctctagaagaggaaataaaagctgctttgcactctg

SEQ ID NO: 3 - DNA, 21 bp, Artificial
  siRNA Target
aagcatttatttgtcaataaa

SEQ ID NO: 4 - DNA, 21 bp, Artificial
  siRNA Target
caggagtgtcataatgctgta

SEQ ID NO: 5 - DNA, 21 bp, Artificial
  siRNA Target
ctcacgtatgatgaagttaaa

SEQ ID NO: 6 - DNA, 21 bp, Artificial
  siRNA Target
ctccatcatattatttgtgta

SEQ ID NO: 7 - DNA, 21 bp, Artificial
  siRNA Target
ccgcactgtcttgtcaagata

SEQ ID NO: 8 - DNA, 21 bp, Artificial
  siRNA Target
ctggtcctctttgctcagaaa

SEQ ID NO: 9 - DNA, 21 bp, Artificial
  siRNA Target
cacgtgtaatctgctatgaaa

SEQ ID NO: 10 - DNA, 21 bp, Artificial
  siRNA Target
aagcatgatctccaagttaaa

SEQ ID NO: 11 - DNA, 21 bp, Artificial
  siRNA Target
cagggtctgcatgacccacaa

SEQ ID NO: 12 - DNA, 21 bp, Artificial
  siRNA Target
cagcgctgatttgaacaataa

SEQ ID NO: 13 - DNA, 21 bp, Artificial
  siRNA Target
aaccagagtatttatatctaa

SEQ ID NO: 14 - DNA, 21 bp, Artificial
  siRNA Target
tccaaatttataaatattaaa

SEQ ID NO: 15 - DNA, 21 bp, Artificial
  siRNA Target
acgggagaatattacaagcaa

SEQ ID NO: 16 - DNA, 21 bp, Artificial
  siRNA Target
cagcatcaagtccgttaataa

SEQ ID NO: 17 - DNA, 21 bp, Artificial
  siRNA Target
gaggatgtgattgatattgaa

SEQ ID NO: 18 - DNA, 21 bp, Artificial
  siRNA Target
atgctggaatttgaagctaaa

SEQ ID NO: 19 - DNA, 21 bp, Artificial
  siRNA Target
cacctcctggaaagtccacaa

SEQ ID NO: 20 - DNA, 21 bp, Artificial
  siRNA Target
gtgggaaagtatgttcagcaa

SEQ ID NO: 21 - DNA, 21 bp, Artificial
  siRNA Target
agccgtggatgccaaagacta

SEQ ID NO: 22 - DNA, 21 bp, Artificial
  siRNA Target
caggtgtttgccaaacctcaa

SEQ ID NO: 23 - DNA, 21 bp, Artificial
  siRNA Target
ctggttgactaccttgaaata

SEQ ID NO: 24 - DNA, 21 bp, Artificial
  siRNA Target
tacgaactggccatcaatata

SEQ ID NO: 25 - DNA, 21 bp, Artificial
  siRNA Target
cagactgacattccaaacaaa

SEQ ID NO: 26 - DNA, 21 bp, Artificial
  siRNA Target
ttggacaaagaaagtctttaa

SEQ ID NO: 27 - DNA, 21 bp, Artificial
  siRNA Target
atccaagaagtcacaaatcaa

SEQ ID NO: 28 - DNA, 21 bp, Artificial
  siRNA Target
aagaagcgctttgaagttaaa

SEQ ID NO: 29 - DNA, 21 bp, Artificial
  siRNA Target
ctgggacattgtggttgataa

SEQ ID NO: 30 - DNA, 21 bp, Artificial
  siRNA Target
atgcagcaatctctagaacaa

SEQ ID NO: 31 - DNA, 21 bp, Artificial
  siRNA Target
caaagttgacattgtaaataa

SEQ ID NO: 32 - DNA, 21 bp, Artificial
  siRNA Target
aacatgatacttgttcatgaa

SEQ ID NO: 33 - DNA, 21 bp, Artificial
  siRNA Target
aacattaaagtgtatggtaaa

SEQ ID NO: 34 - protein, 21 aa, Artificial
  siRNA Target
CEKARESLVSTFRARPRGRHF